SEQUENCE LISTING (23 sequences)

SEQ ID NO: 1 - protein, 373 aa, Zebrafish
MMLLKLKWTSSGVKDTPHTLLSIIITFCVFFMLEIVDANSWWSLAMNPLLIPEVYVIGGQPLCSQLSGLSKGQKKLCQLYQDHMQYIGEGAKTGIRECQHQFRHRRWNCSTVDNSTVLGRVMHIGSRESAFAFAISAAGVLHAVSRACREGALSSCGCSRASRPKDLPRDWLWGGCGDNLNYGYRFSREFVDAREREKTFSKGSAESARQMMNLHNNEAGRRIVSDLADVSCKCHGVSGSCSLKTCWLQLADFRKVGDVLKEKYDSAAAMRMNGRGKLVQMHSKFSPPSGQDLLYLQPSPDYCIRNSSSGSLGTQGRLCNKTSEGMDGCALMCCGRGYDQYKAELVERCHCKFHWCCYVRCKRCSSIVDQYVC

SEQ ID NO: 2 - protein, 380 aa, Homo sapien
MKKSIGILSPGVALGMAGSAMSSKFFLVALAIFFSFAQVVIEANSWWSLGMNNPVQMSEVYIIGAQPLCSQLAGLSQGQKKLCHLYQDHMQYIGEGAKTGIKECQYQFRHRRWNCSTVDNTSVFGRVMQIGSRETAFTYAVSAAGVVNAMSRACREGELSTCGCSRAARPKDLPRDWLWGGCGDNIDYGYRFAKEFVDARERERIHAKGSYESARILMNLHNNEAGRRTVYNLADVACKCHGVSGSCSLKTCWLQLADFRKVGDALKEKYDSAAAMRLNSRGKLVQVNSRFNSPTTQDLVYIDPSPDYCVRNESTGSLGTQGRLCNKTSEGMDGCELMCCGRGYDQFKTVQTERCHCKFHWCCYVKCKKCTEIVDQFVCK

SEQ ID NO: 3 - protein, 379 aa, Mus musculus
MKKPIGILSPGVALGTAGGAMSSKFFLMALATFFSFAQVVIEANSWWSLGMNNPVQMSEVYIIGAQPLCSQLAGLSQGQKKLCHLYQDHMQYIGEGAKTGIKECQYQFRHRRWNCSTVDNTSVFGRVMQIGSRETAFTYAVSAAGVVNAMSRACREGELSTCGCSRARPKDLPRDWLWGGCGDNIDYGHPFAKEFVDARERERIHAKGSYESARILMNLHNNEAGRRTVYNLADVACKCHGVSGSCSLKTCWLQLADFRKVGDALKEKYDSAAAMRLNSRGKLVQVNSRFNSPTTQDLVYIDPSPDYCVRNESTGSLGTQGRLCNKTSEGMDGCELMCCGRGYDQFKTVQTERCHCKFHWCCYVKCKKCTEIVDQFVCK

SEQ ID NO: 4 - protein, 380 aa, Xenopus
MRKNLWTFQFGGEASGLVGSAMVSQHFVVLLMSLYCLTQSVVESSSWWSLGMNPVQMPEVYIIGAQPLCSQLSGLSQGQKKLCQLYQDHMQFIGDGAKTGIKECQYQFRHRRWNCSTVDNTSVFGRVMQIGSRETAFTYAISAAGVVNAVSRACREGELSTCGCSRAARPKDLPRDWLWGGCGDNLDYGYRFAKEFVDAREREKIHQKGSYESSRIMMNLHNNEAGRRAVSTLADVACKCHGVSGSCSLKTCWLQLADFRKVGDHLKEKYDSAGAMKLNTRGKLVQVNNKFNSPTMNDLVYIDPSPDYCVHNESTGSLGTQGRLCNKTSEGMDGCELMCCGRGYDQFKTVQTERCHCKFHWCCYVKCKKCTEVVDQFACK

SEQ ID NO: 5 - protein, 385 aa, Chick
MEKSTAVLIQGGALGTLGSTMASQYLVVALAIFSSCTQVVIEASSWWSLGMNPMNPMNPVQMSEVYIIGAQPLCSQLAGLSQGQKKLCQLYQDHMQFIGEGAKTGIKECQYQFRHRRWNCSTVDNNSVFGRVMQIGSRETAFTYAVSAAGVVNAMSRACREGELSSCGCSRAARPKDLPRDWLWGGCGDNIEYGYRFAKEFVDARERERVYQRGSYESARIMMNLHNNEAGRRTVYNLADVACKCHGVSGSCSLKTCWLQLADFRKVGDALKEKYDSAAAMKLNSRGKLVQMNSRFNAPTIHDLIYIDPSPDYCMRNESTGSLGTQGRLCNKTSEGMDGCELMCCGRGYDQFKTVQRERCHCKFHWCCYVKCKLCTEIVDQFVCK

SEQ ID NO: 6 - protein, 359 aa, Axolotl
MATTHLTAALALLCALLQVDIEASSWWSLAMNPVQIPEAYIVGAQPLCSQLPGLSPGQKKLCQLYQDHMPYIGEGAKTGIKECQYQFRHRRWNCSTVDNASVFGRVMQIGSRETAFTYSISAAGVVNAVSRACREGELSTCGCSRAARPKDLQRDWLWGGCGDNLEYGYRFAKEFVDAREREKIHTKGSYESSRTLMNIHNNEAGRRTVYNLADAACKCHGVSGSCSLKTCWLQLADFRKVGDFLKEKYDSAASMRLNARGKLVQVNSRFNPPTTNDLVYVDTSPDYCVRNESTGSLGTQGRLCNKTSEGMDGCELMCCGRGYDQFKTVQTERCHCKFHWCCYVKCKKCTEIVDQFVCK

SEQ ID NO: 7 - protein, 363 aa, Zebrafish
MDVRMNQGHLLLAVTLIVCNSQLLVVANSWWSLAMNPIQRPEMYIIGAQPLCSQLTGLSQGQRKLCQLYQDHMVYIGEGAKTGIKECQYQFRQRRWNCSTVDNTSVFGRVMHIGSRETAFTYAVSAAGVVNAVSRACREGELSTCGCSRAARPRDLPRDWLWGGCGDNVNYGYRFAREFVDAREREKNYPRGSVEHARTLMNLQNNEAGRMAVYNLANVACKCHGVSGSCSLKTCWLQLADFRRVGEFLKEKYDSAAAMRINRRGKLELVNNRFNPPTGEDLVYIDPSPDYCLRNETTGSLGTQGRLCNKTSEGMDGCELMCCGRGYDQFKTYKHERCHCKFHWCCYVKCKRCTSLVDQFVCK

SEQ ID NO: 8 - protein, 359 aa, Homo sapiens
MPSLLLLFTAALLSSWAQLLTDANSWWSLALNPVQRPEMFIIGAQPVCSQLPGLSPGQRKLCQLYQEHMAYIGEGAKTGIKECQHQFRQRRWNCSTADNASVFGRVMQIGSRETAFTHAVSAAGVVNAISRACREGELSTCGCSRTARPKDLPRDWLWGGCGDNVEYGYRFAKEFVDAREREKNFAKGSEEQGRVLMNLQNNEAGRRAVYKMADVACKCHGVSGSCSLKTCWLQLAEFRKVGDRLKEKYDSAAAMRVTRKGRLELVNSRFTQPTPEDLVYVDPSPDYCLRNESTGSLGTQGRLCNKTSEGMDGCELMCCGRGYNQFKSVQVERCHCKFHWCCFVRCKKCTEIVDQYICK

SEQ ID NO: 9 - protein, 372 aa, Mus musculus
MLVPGHWDGLRPAMPSLLLVVVAALLSSWAQLLTDANSWWSLALNPVQRPEMFIIGAQPVCSQLPGLSPGQRKLCQLYQEHMSYIGEGAKTGIRECQHQFRQRRWNCSTVDNTSVFGRVMQIGSRETAFTYAVSAAGVVNAISRACREGELSTCGCSRAARPKDLPRDWLWGGCGDNVEYGYRFAKEFVDAREREKNFAKGSEEQGRALMNLQNNEAGRRAVYKMADVACKCHGVSGSCSLKTCWLQLAEFRKVGDRLKEKYDSAAAMRITRQGKLELANSRFNQPTPEDLVYVDPSPDYCLRNETTGSLGTQGRLCNKTSEGMDGCELMCCGRGYDRFKSVQVERCHCRFHWCCFVRCKKCTEVVDQYVCK

SEQ ID NO: 10 - protein, 360 aa, Xenopus
MTPILRLLLLSSLLSCWKQSVVGANSWWSMALNPVQRPEMFIIGAQPLCSQLTGLSPGQRKLCQLYQDHMVHIGEGAKTGIKECQHQFKHRRWNCSTVDNNSVFGRVMQIGSREASFTYAISSAGVVNAISRACREGELSTCGCSRTPRPKDLPRDWLWGGCGDNVEYGYRFAKEFVDAREREKNFPKGSEEQARSLMNLQNNEAGRRAVYKLADVACKCHGVSGSCSLKTCWLQLADFRKVGEYIKEKYDSAASMRLNKRNKLEQVNQRFNPPTGEDLVYLDPSPDYCLYNETTGSLGTHGRQCNKTSEGMDGCELMCCGRGYDQFKTVQVERCHCKFQWCCFVKCKKCTEIVDQFVCK

SEQ ID NO: 11 - protein, 357 aa, Axolotl
MPGIRLLLAAALLCCPPPAGATSWWSLAQNPVQRPEMYIIGAQPVCSQLSSLSPGQKKLCQLYQDHMMYIGEGAKTGIKECQYQFKQRRWNCSTVDNTSVFGRVMQIGSRETAFTYAVSAAGVVNAISRACREGELSTCGCSRTTRPKDLHRDWLWGGCGDNVDYGYRFAKEFVDAREREKNYPKGSEEQARTLMNLQNNEAGRRAVYKLADAACKCHGVSGSCSLKTCWLQLADFRKVGDHLKEKYDSAAAMRINRKGKLELVNNRFNLPTVEDLVYTDQSPDYCLRNESTGSLGTLGRLCNKTSEGMDGCELMCCGRGYDQFKTVQVERCHCKFHWCCFVKCKKCTEIVDQYVCK

SEQ ID NO: 12 - DNA, 25 bp, Artificial
  primer
gcagatttggtatattggaattcat

SEQ ID NO: 13 - DNA, 25 bp, Artificial
  primer
ctagaacatccttgtaaagctcagg

SEQ ID NO: 14 - DNA, 20 bp, Artificial
  primer
actttgacttcgccttcctg

SEQ ID NO: 15 - DNA, 20 bp, Artificial
  primer
caccttcatgagctccacct

SEQ ID NO: 16 - DNA, 25 bp, Artificial
  primer
attcactccaggatgagacttcata

SEQ ID NO: 17 - DNA, 24 bp, Artificial
  primer
gtttctgttgtgggctttgattag

SEQ ID NO: 18 - DNA, 20 bp, Artificial
  primer
ggtatgggacagaaagacag

SEQ ID NO: 19 - DNA, 20 bp, Artificial
  primer
agagtccatcacgataccag

SEQ ID NO: 20 - DNA, 22 bp, Artificial
  primer
cgctattggagctggaattacc

SEQ ID NO: 21 - DNA, 20 bp, Artificial
  primer
gaaacggctaccacatccaa

SEQ ID NO: 22 - DNA, 20 bp, Artificial
  primer
cagcttctctcacggcttgg

SEQ ID NO: 23 - DNA, 21 bp, Artificial
  primer
aaagctcaggaactcgctctg

(19) United States
(12) Patent Application Publication
(10) Pub. No.: US 2009/0047276 A1
(43) Pub. Date: Feb. 19, 2009

(54) METHODS FOR INCREASING CELL OR TISSUE REGENERATION IN A VERTEBRATE SUBJECT

(75) Inventors: Randall T. MOON, Kenmore, WA (US); Cristi Lee Stoick COOPER, Seattle, WA (US); Gilbert WEIDINGER, Dresden (DE)

Correspondence Address: NIXON PEABODY LLP - PATENT GROUP, 1100 CLINTON SQUARE, ROCHESTER, NY, 14604, US

(73) Assignee: UNIVERSITY OF WASHINGTON, Seattle, WA (US)

(21) Appl. No.: 12/120,422

(22) Filed: May 14, 2008

Related U.S. Application Data
(60) Provisional application No. 60/938,161, filed on May 15, 2007.

(51) Int. Cl. A61K 39/395; A61K 38/02; A61K 38/17; A61K 31/7088

(52) U.S. Cl. 424/130.1; 514/2; 514/44; 514/12

(57) ABSTRACT

A method is provided for increasing the activity of adult and embryonic stem cells, progenitor cells and/or differentiated cells in vivo in a vertebrate subject. Methods are provided for increasing cell or tissue regeneration in a vertebrate subject by administering one or more Wnt/β-catenin signal-promoting agents and/or one or more inhibitors of β-catenin-independent signaling to the vertebrate subject in need thereof.

This application claims the benefit of U.S. Provisional Patent Application Ser. No. 60/938,161, filed May 15, 2007, which is hereby incorporated by reference in its entirety.

### STATEMENT OF GOVERNMENT SUPPORT

This invention was made by government support of The National Institutes of Health Grant No. 5R01GM073887-03 and PO1 HL03174. The Government has certain rights in this invention.

### FIELD

The invention generally relates to methods for increasing the activity of embryonic and/or adult stem cells, progenitor cells, mesenchymal progenitor/stem cells, or differentiated cells in vivo in a vertebrate subject. The invention further relates to methods for increasing cell or tissue regeneration in a vertebrate subject by administering one or more Wnt/β-catenin signal-promoting agents and/or one or more inhibitors of β-catenin-independent signaling to the vertebrate subject in need thereof.

### BACKGROUND

All organisms mount a biological response to damage, but they vary widely in their ability to recover. Humans constantly renew components of blood, skeletal muscle and epithelia. Such homeostatic renewal is thought to be mediated by resident stem cells of a specific lineage. In contrast, while humans can regenerate an injured liver and repair limited insults to bone, muscle, digit tips and cornea, they do not regenerate the heart, spinal cord, retina or limbs. Thus, humans and other mammals are quite disadvantaged when compared to amphibians and teleost fish, which have a remarkable capacity to regenerate damaged organs including heart, spinal cord, retina, and limbs/fins. Akimenko et al., Dev Dyn 226:190-201, 2003; Brockes, J. P. and Kumar, A., Nat Rev Mol Cell Biol 3:566-74, 2002; Poss et al., Dev Dyn 226:202-10, 2003; Poss et al., Science 298:2188-90, 2002. A dramatic example of organ regeneration is that of amphibian limbs and fish fins, where intricate structures consisting of multiple cells types that are patterned into complex tissues are faithfully restored after amputation. The mechanisms that enable lower vertebrates to re-establish such structures and the reasons why mammals are not able to do so, are incompletely understood. Elucidation of these mechanisms and an understanding of why regenerative capacity has diminished in vertebrate evolution hold the potential to revolutionize clinical medicine, with practical applications ranging from organ disease and wound treatment to possible alternatives to prosthetics for amputees.

While repair of many organs, such as the chicken retina or mouse liver, is thought to be mediated through activation of resident stem cells or proliferation of normally quiescent differentiated cells, respectively (Fausto et al., Hepatology 43:S45-53, 2006; Fischer, A. J. and Reh, T. A., Nat Neurosci 4:247-52, 2001), amphibian and fish appendages regenerate through a process termed “epimorphic regeneration”, sometimes called “true” regeneration. This occurs in three steps: first, wound healing and formation of the wound epidermis; second, formation of a regeneration blastema, a population of mesenchymal progenitor cells that is necessary for proliferation and patterning of the regenerating limb/fin; and third, regenerative outgrowth and pattern reformation (Akimenko et al., Dev Dyn 226:190-201, 2003; Poss et al., Dev Dyn 226:202-10, 2003). Progenitor cells of the blastema in the regenerating axolotl tail can be formed by reprogramming and de-differentiation of differentiated cells (Casimir et al., Development 104:657-68, 1988; Echeverri et al., Dev Biol 236:151-64, 2001; Echeverri and Tanaka, Science 298:1993-6, 2002; Kintner, C. R. and Brockes, J. P., Nature 308:67-9, 1984; Lentz, T. L., Am J Anat 124:447-79, 1969; Lo et al., Proc Natl Acad Sci 90:7230-4, 1993). These cells express transcriptional repressors of the msx gene family that may help maintain a pluripotent state (Akimenko et al., Development 121:347-57, 1995; Yokoyama et al., Dev Biol 233:72-9, 2001). Recently, activation of resident muscle stem cells has also been reported to occur in regenerating salamander limbs (Morrison et al, J Cell Biol 172:433-40, 2006). Thus, it is likely that de-differentiation and stem cell activation both contribute to formation of the blastema. While de-differentiation of cells has not yet been shown to occur in regenerating structures other than amphibian limbs and tails, the morphology, ontology and gene expression profile of the zebrafish blastema in the regenerating tail fin suggest that zebrafish tail regeneration occurs by similar mechanisms.

A major question that remains incompletely answered involves the identification of the extracellular signals that regulate formation or activation of stem cells during regeneration. While hedgehog signaling has been implicated in newt tail and chick retina regeneration (Schnapp et al., Development 132:3243-53, 2005; Spence et al., Development 131:4607-21, 2004) and BMP signaling in newt lens and Xenopus tail regeneration (Beck et al., Dev Cells 5:429-39, 2003; Grogg et al., Nature 438:858-62, 2005), the strongest evidence to date points to FGF signaling as an essential regulator of progenitor cell formation in limb and fin regeneration. FGF-10 is sufficient to re-activate regeneration in Xenopus limbs at later stages of development where limbs have lost their regenerative capacity (Yokoyama et al., Dev Biol 233:72-9, 2001), and FGF-2 soaked beads can stimulate chick limbs, which do not regenerate, to do so (Taylor et al., Dev Biol 163:282-4, 1994). Inhibition of FGF signaling by pharmacological inhibitors or expression of a dominant-negative FGF receptor blocks blastema formation in zebrafish caudal fin regeneration (Lee et al., Development 132:5173-83, 2005; Poss et al., Dev Biol 222:347-58, 2000) and a mutation in zebrafish fgf20a causes an early block in blastema formation (Whitehead et al., Science 310:1957-60, 2005).

Wnt/β-catenin signaling regulates progenitor cell fate and proliferation during embryonic development and in adult tissue homeostasis (Logan, C. Y. and Nusse, R., Annu Rev Cell Dev Biol 20:781-810, 2004; Reya, T. and Clevers, H., Nature 434:843-50, 2005), raising the possibility that it is also involved in progenitor cell function during regeneration. Several studies have documented expression of Wnt ligands and components of the β-catenin signaling pathway in regenerating amphibian and fish appendages (Caubit et al., Dev Dyn 210:1-10, 1997a; Caubit et al., Dev Dyn 208:139-48, 1997; Poss et al., Dev Dyn 219:282-6, 2000) and other studies have suggested that Wnt/β-catenin signaling is functionally involved in proliferation of cells during regeneration of mammalian muscle, liver and bone (Polesskaya et al., Cell 113:841-52, 2003; Sodhi et al., J Hepatol 43:132-41, 2005; Zhong et al., Bone 39:5-16, 2006). However, whether Wnt/β-catenin signaling plays an essential role in the epimorphic, “true”, regeneration of complex structures has not been tested.

Many Wnt ligands can activate β-catenin-independent (“noncanonical”) signaling pathways (Slusarski et al., Dev Biol 182:114-20, 1997; Veeman et al., Dev Cells 5:367-77, 2003) that are well documented to regulate cell polarity and cell migration during embryonic development (Veeman et al., Dev Cells 5:367-77, 2003). However, other than reports which indicate that β-catenin-independent Wnt signaling might act to suppress tumor formation (Dejmek et al., Cancer Res 65:9142-6, 2005; Jonsson et al., Cancer Res 62:409-16, 2002; Kremenevskaja et al., Oncogene 24:2144-54, 2005, nothing is known about its role in adults. A need exists in the art for improved treatment of tissue degenerative diseases and therapies providing cell or tissue regeneration in a vertebrate subject.

### SUMMARY

The present invention relates a method for increasing cell or tissue regeneration in a vertebrate subject. The invention relates to methods for increasing the successful activity of embryonic and/or adult stem cells, progenitor cells, mesenchymal progenitor/stem cells and/or differentiated cells in vivo in a vertebrate subject. The invention further relates to methods for increasing cell or tissue regeneration in a vertebrate subject by administering one or more Wnt/β-catenin signal-promoting agents and/or one or more inhibitors of β-catenin-independent signaling to the vertebrate subject in need thereof, and increasing in vivo a stem cell, progenitor cell, and/or differentiated cell population in the vertebrate subject compared to the stem cell, progenitor cell, and/or differentiated cell population in the vertebrate subject before treatment, to increase cell or tissue regeneration in the vertebrate subject.

A method for increasing cell or tissue regeneration in a vertebrate subject is provided which comprises administering one or more Wnt/β-catenin signal-promoting agents to the vertebrate subject, increasing in vivo a stem cell, progenitor cell, or differentiated cell population in the vertebrate subject compared to the stem cell, progenitor cell, or differentiated cell population in the vertebrate subject before treatment, to increase cell or tissue regeneration in the vertebrate subject. The Wnt/β-catenin signal-promoting agent is an agonist of one or more of Wnt1, Wnt2, Wnt2b/13, Wnt3, Wnt3a, Wnt4, Wnt6, Wnt7a, Wnt7b, Wnt7c, Wnt8, Wnt8a, Wnt8b, Wnt8c, Wnt10a, Wnt10b, Wnt11, Wnt14, Wnt15, Wnt16, or fgf20a. In one aspect, the Wnt/β-catenin signal-promoting agent is an agonist of Wnt10a. In a further aspect, the Wnt/β-catenin signal-promoting agent is an agonist of Wnt3a or Wnt8. The cell or tissue regeneration can occur in tissues including but not limited to, bone, chondrocytes/cartilage, muscle, skeletal muscle, cardiac muscle, pancreatic cells, endothelial cells, vascular endothelial cells, adipose cells, liver, skin, connective tissue, hematopoietic stem cells, neonatal cells, umbilical cord blood cells, fetal liver cells, adult cells, bone marrow cells, peripheral blood cells, erythroid cells, granulocyte cells, macrophage cells, granulocyte-macrophage cells, B cells, T cells, multipotent mixed lineage colony types, embryonic stem cells, mesenchymal progenitor/stem cells, mesodermal progenitor/stem cells, neural progenitor/stem cells, or nerve cells. The vertebrate can be mammalian, avian, reptilian, amphibian, osteichthyes, or chondrichthyes. In one aspect, the Wnt/β-catenin signal-promoting agent is a glycogen synthase kinase (GSK) inhibitor. In a detailed aspect, the GSK inhibitor is a GSK-3 inhibitor or a GSK-3β inhibitor. The Wnt/β-catenin signal-promoting agent includes, but is not limited to, a polypeptide, peptide mimetic, nucleic acid, small chemical molecule, antisense oligonucleotide, ribozyme, RNAi construct, siRNA, shRNA, or antibody. The Wnt/β-catenin signal-promoting agent is a polypeptide or peptide mimetic. The Wnt signal- or β-catenin signal-promoting agent is a wnt polypeptide, a dishevelled polypeptide, or a β-catenin polypeptide, or peptide mimetic thereof. Increasing the stem cell, progenitor cell, or differentiated cell population in the vertebrate subject can be a result of cell proliferation, cell homing, decreased apoptosis, self renewal, or increased cell survival.

A method for increasing cell or tissue regeneration in a vertebrate subject comprising is provided which comprises administering one or more antagonists of β-catenin-independent signaling to the vertebrate subject, increasing in vivo a stem cell, progenitor cell, or differentiated cell population in the vertebrate subject compared to the stem cell, progenitor cell, or differentiated cell population in the vertebrate subject before treatment, to increase cell or tissue regeneration in the vertebrate subject. In one aspect, the antagonist of β-catenin-independent signaling is an antagonist of Wnt5a. In a further aspect, the antagonist of β-catenin-independent signaling is an antagonist of Wnt5b. The antagonist of β-catenin-independent signaling can increase Wnt/β-catenin signaling. In one aspect, cell or tissue regeneration can occur in bone, chondrocytes/cartilage, muscle, skeletal muscle, cardiac muscle, pancreatic cells, endothelial cells, vascular endothelial cells, adipose cells, liver, skin, connective tissue, hematopoietic stem cells, neonatal cells, umbilical cord blood cells, fetal liver cells, adult cells, bone marrow cells, peripheral blood cells, erythroid cells, granulocyte cells, macrophage cells, granulocyte-macrophage cells, B cells, T cells, multipotent mixed lineage colony types, embryonic stem cells, mesenchymal progenitor/stem cells, mesodermal progenitor/stem cells, neural progenitor/stem cells, or nerve cells. The vertebrate can be mammalian, avian, reptilian, amphibian, osteichthyes, or chondrichthyes. The antagonist of β-catenin-independent signaling includes, but is not limited to, a polypeptide, peptide mimetic, nucleic acid, small chemical molecule, antisense oligonucleotide, ribozyme, RNAi construct, siRNA, shRNA, or antibody. In a further aspect, the antagonist of β-catenin-independent signaling is a polypeptide or peptide mimetic. The antagonist of β-catenin-independent signaling can be a polypeptide or peptide mimetic of Wnt5a or Wnt5b. Increasing the stem cell, progenitor cell, or differentiated cell population in the vertebrate subject can be a result of cell proliferation, cell homing, decreased apoptosis, self renewal, or increased cell survival.

### BRIEF DESCRIPTION OF THE DRAWINGS

FIGS. 1A, 1B, and 1C show Wnt/β-catenin signaling is upregulated in regenerating zebrafish tail fins.

FIGS. 2A, 2B, and 2C show Wnt/β-catenin signaling is required for zebrafish tail fin regeneration.

FIGS. 3A, 3B, 3C, and 3D show Wnt/β-catenin signaling regulates specification and proliferation of the regeneration blastema.

FIGS. 4A, 4B, 4C, 4D, 4E, and 4F show β-catenin-dependent and β-catenin-independent Wnt signaling pathways have opposing roles in zebrafish fin regeneration.

FIGS. 5A and 5B show fins regenerate faster in fish heterozygous for a loss-of-function mutation in axin1.

FIGS. 6A and 6B show fins regenerate faster in wnt5b mutant fish.

FIGS. 7A, 7B, and 7C show Wnt/β-catenin regulates FGF signaling during fin regeneration.

FIG. 8 shows a model of signaling events regulating zebrafish fin regeneration.

FIGS. 9A, 9B, and 9C show Wnt/β-catenin signaling is upregulated in regenerating zebrafish heart and mouse liver.

FIGS. 10A and 10B show the previously described zebrafish wnt5 homolog pipetail (ppt) is the ortholog of wnt5b, while a newly cloned homolog likely represents the zebrafish ortholog of wnt5a.

FIG. 11A through 11M show establishment of heat-shock inducible Dickkopf1GFP and heat-shock inducible Wnt5bGFP transgenic zebrafish lines.

FIG. 12 shows wnt5b expression during fin regeneration is regulated by Wnt/β-catenin signaling.

FIG. 13 shows heat-shock induced expression of Dkk1 represses fgf20a expression during fin regeneration.

### DETAILED DESCRIPTION

The present invention relates a method for increasing cell or tissue regeneration in a vertebrate subject. The invention relates to methods for increasing the successful activity of embryonic and/or adult stem cells, progenitor cells, mesenchymal progenitor/stem cells, or differentiated cells in vivo in a vertebrate subject. The invention further relates to methods for increasing cell or tissue regeneration in a vertebrate subject by administering one or more Wnt/β-catenin signal-promoting agents and/or one or more inhibitors of β-catenin-independent signaling to the vertebrate subject in need thereof, and increasing in vivo a stem cell, progenitor cell population, or differentiated cell in the vertebrate subject compared to the stem cell or progenitor cell, or differentiated cell population in the vertebrate subject before treatment, to increase cell or tissue regeneration in the vertebrate subject. A method for increasing stem cell or progenitor cell population is provided to repair or replace damaged tissue in a vertebrate subject, wherein the cell or tissue regeneration occurs in bone, chondrocytes/cartilage, muscle, skeletal muscle, cardiac muscle, pancreatic cells, endothelial cells, vascular endothelial cells, adipose cells, liver, skin, connective tissue, hematopoietic stem cells, neonatal cells, umbilical cord blood cells, fetal liver cells, adult cells, bone marrow cells, peripheral blood cells, erythroid cells, granulocyte cells, macrophage cells, granulocyte-macrophage cells, B cells, T cells, multipotent mixed lineage colony types, embryonic stem cells, mesenchymal progenitor/stem cells, mesodermal progenitor/stem cells, neural progenitor/stem cells, or nerve cells.

In contrast to mammals, lower vertebrates have a remarkable capacity to regenerate complex structures damaged by injury or disease. This process, termed epimorphic regeneration, involves progenitor cells created through the reprogramming of differentiated cells or through the activation of resident stem cells. Wnt/β-catenin signaling regulates progenitor cell fate and proliferation during embryonic development and stem cell function in adults, but its functional involvement in epimorphic regeneration has not been addressed. Using transgenic fish lines, embodiments of the present invention demonstrate that Wnt/β-catenin signaling is activated in the regenerating zebrafish tail fin and required for formation of the “regeneration blastema”, a structure that is formed after amputation, which consists of progenitor cells that proliferate and give rise to the regenerating fine. The invention also shows that Wnt/β-catenin signaling appears to act upstream of FGF signaling, which has recently been found to be essential for fin regeneration. Another important aspect of this invention is the discovery that increased Wnt/β-catenin signaling is sufficient to augment regeneration, since tail fins regenerate faster in fish heterozygous for a loss of function mutation in axin1, a negative regulator of the pathway. The invention also relates to the discovery that activation of Wnt/β-catenin signaling by overexpression of wnt8 increases proliferation of progenitor cells in the regenerating fin and that, in contrast, overexpression of wnt5b/pipetail reduces expression of Wnt/β-catenin target genes, impairs proliferation of progenitors, and inhibits fin regeneration. Importantly, this invention also relates to the discovery that fin regeneration is accelerated in wnt5b mutant fish. These data suggest that Wnt/β-catenin signaling promotes regeneration while a distinct pathway activated by wnt5b/pipetail acts in a negative feedback to limit regeneration. Evidence is provided that both β-catenin-dependent and β-catenin-independent Wnt signaling pathways regulate zebrafish fin regeneration and can be modulated to enhance regeneration.

It is to be understood that this invention is not limited to particular methods, reagents, compounds, compositions or biological systems, which can, of course, vary. It is also to be understood that the terminology used herein is for the purpose of describing particular embodiments only, and is not intended to be limiting. As used in this specification and the appended claims, the singular forms “a”, “an” and “the” include plural referents unless the content clearly dictates otherwise. Thus, for example, reference to “a cell” includes a combination of two or more cells, and the like.

The term “about” as used herein when referring to a measurable value such as an amount, a temporal duration, and the like, is meant to encompass variations of ±20% or ±10%, more preferably ±5%, even more preferably ±1%, and still more preferably +0.1% from the specified value, as such variations are appropriate to perform the disclosed methods.

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

“Increasing a stem cell or progenitor cell population in vivo in a vertebrate subject” or “increasing stem cell or progenitor cell population in a vertebrate subject” or “increasing the successful activity of progenitor/stem cells in vivo in a mammalian subject” or “increasing successful activity of progenitor/stem cell” refers to increasing an aspect of the life cycle of the progenitor/stem cell for example, as a result of a process, including but not limited to, cell proliferation, cell homing to the desired target tissue (e.g., transplanted progenitor/stem cells are provided intravenously and become established in the bone marrow), decreased apoptosis, self renewal, or increased cell survival. Increasing stem cell or progenitor cell population can occur by a variety of cellular mechanisms including, but not limited to, by means of activating or promoting progenitor/stem cell function, by promoting differentiation of progenitor/stem cells, or a combination thereof, to repair or replace damaged tissue in a vertebrate subject. An increase in progenitor/stem cells in vivo can be measured by a cellular assay as disclosed herein (e.g., in vivo hematopoietic stem cell repopulation assay, or hematopoeitic colony-forming unit (CFU) assay) or other cellular assay known in the art. Increased progenitor/stem cells in vivo in a vertebrate subject or increased successful activity can be measured by comparing the fold increase in the cells of interest in a vertebrate subject treated by administering one or more Wnt/β-catenin pathway agonist and/or one or more inhibitors of β-catenin-independent signaling to the vertebrate subject compared to the cells of interest in a vertebrate subject in the absence of such treatment, as measured by any of the cellular activity assays for progenitor/stem cells discussed herein or known to one skilled in the art. The baseline number of progenitor/stem cells in a vertebrate subject is considered the number of these cells in a vertebrate subject in the absence of such treatment. An increase in the cells of interest in the treated vertebrate subject by administering one or more Wnt/β-catenin pathway agonist and/or one or more inhibitors of β-catenin-independent signaling can be, for example, at least 1.5 fold, at least 2-fold, at least 4-fold, at least 8-fold, or at least 10-fold compared to the cells of interest in the vertebrate subject before treatment.

“Increasing cell or tissue regeneration in a vertebrate subject” refers to the capacity in vertebrates to regenerate complex structures, appendages or organs damaged by injury or disease. Methods and compositions of the present invention show that increased Wnt/β-catenin signaling and/or inhibition of β-catenin-independent signaling in regenerating zebrafish tail fin enhances proliferation of progenitor cells of the blastema and leads to faster regeneration of an amputated fin.

“Increasing in vivo mesenchymal progenitor/stem cells in a vertebrate subject” or “increasing mesenchymal progenitor/stem cells in a vertebrate subject” or “increasing the successful activity of mesenchymal progenitor/stem cells in vivo in a vertebrate subject” refers to increasing an aspect of the life cycle of the mesenchymal progenitor/stem cells, for example, as a result of a cellular process, including but not limited to, cell proliferation, cell homing to the desired target tissue, decreased apoptosis, self renewal, or increased cell survival. An increase in mesenchymal progenitor/stem cells in vivo can be measured by a cellular assay as disclosed herein (e.g., in vivo stem cell or progenitor cell repopulation assay, stem cell or progenitor cell colony-forming unit (CFU) assay or other cellular assay known in the art). Increased mesenchymal progenitor/stem cells in vivo in a vertebrate subject can be measured by comparing the fold increase in mesenchymal progenitor/stem cells in a vertebrate subject treated by administering one or more Wnt/β-catenin pathway agonist(s) and/or one or more inhibitor(s) of β-catenin-independent signaling to the vertebrate subject compared to the number of mesenchymal progenitor/stem cells in a vertebrate subject in the absence of such treatment. The increase in mesenchymal progenitor/stem cells in the vertebrate subject treated by administering one or more Wnt/β-catenin pathway agonist(s) and/or one or more inhibitor(s) of β-catenin-independent signaling can be, for example, at least 1.5 fold, at least 2-fold, at least 4-fold, at least 8-fold, or at least 10 fold compared to mesenchymal progenitor/stem cells in the vertebrate subject before treatment.

“Interacting one or more Wnt/β-catenin pathway agonist(s) and/or one or more inhibitor(s) of β-catenin-independent signaling with the stem cell or progenitor cell population in the vertebrate subject” refers to either (1) direct contact between the agent and the cell or (2) an indirect interaction between the agent and the cell through an intermediary molecule or intermediary cell type. The interacting step can refer to directly contacting the progenitor/stem cell with the Wnt/β-catenin pathway agonist(s) and/or one or more inhibitor(s) of β-catenin-independent signaling to induce Wnt/β-catenin signaling within the progenitor/stem cell, for example, through receptor/ligand interaction, intracellular signaling, transcriptional regulation of gene expression, cell-cell interaction or intercellular signaling.

Alternatively, the “interacting” step refers to an indirect interaction between a progenitor/stem cell and the Wnt/β-catenin pathway agonist(s) and/or one or more inhibitor(s) of β-catenin-independent signaling mediated through a third component, for example, through an intermediary signaling molecule, receptor, ligand, growth factor, or cell type, that affects, or is affected, by Wnt/β-catenin pathway agonist(s) and/or one or more inhibitor(s) of β-catenin-independent signaling. For example, “interacting one or more Wnt/β-catenin pathway agonist(s) and/or one or more inhibitor(s) of β-catenin-independent signaling with the stem cell or progenitor cell population in the vertebrate subject” can occur by an indirect interaction between stem cells and/or progenitor cells with Wnt/β-catenin pathway agonist(s) and/or one or more inhibitor(s) of β-catenin-independent signaling. The indirect interaction can occur through intermediary signaling molecules for example, growth factors, ligands, receptors or through other cell types that transmit the intermediary signals. The intermediary signaling molecules, growth factors, transcription factors, ligands, or receptors that increase stem cells, progenitor cells or the regenerative potential of any tissue, organ or structure of interest in vivo in a vertebrate subject include, but are not limited to, Notch, Notch1, Jagged-1, Delta, Delta-1, Delta-4, Oct-3/4, Rex-1, Nanog, LIF-STAT2, STAT5, STAT5A, sonic hedgehog, bone morphogenetic proteins, cyclin-dependent kinase inhibitor, p21^{Cip1/Waf1}, HoxB4, or cytokines, e.g., SCF, Fit-3L, G-CSF, IL-3, IL-6 or IL-11. An indirect interaction between the cells of interest and the Wnt/β-catenin pathway agonist(s) and/or one or more inhibitor(s) of β-catenin-independent signaling can occur by contacting the cells of interest with a number of different growth factors or with a number of different cell types.

“Wnt/β-catenin pathway agonist(s)” refers to an agonist of the Wnt signaling pathway, including but not limited to an agonist of one or more of Wnt1, Wnt2, Wnt2b/13, Wnt3, Wnt3a, Wnt4, Wnt5a, Wnt5b, Wnt6, Wnt7a, Wnt7b, Wnt7c, Wnt8, Wnt8a, Wnt8b, Wnt8c, Wnt10a, Wnt10b, Wnt11, Wnt14, Wnt15, or Wnt16, for example, a nucleic acid comprising a nucleotide sequence that encodes a Wnt5b polypeptide or peptide mimetic, a polypeptide comprising an amino acid sequence of a Wnt5b polypeptide or peptide mimetic. “Wnt/β-catenin pathway agonist(s)” also refers to one or more of the following polypeptides or a fragment thereof: a Dkk polypeptide, a crescent polypeptide, a cerberus polypeptide, an axin polypeptide, a Frzb polypeptide, a glycogen synthase kinase polypeptide, a T-cell factor polypeptide, or a dominant negative dishevelled polypeptide, especially when used in a way that activates Wnt/β-catenin signaling.

“Wnt/β-catenin pathway agonist(s) and/or one or more inhibitor(s) of β-catenin-independent signaling” further refers to agonists or antagonists of positive or negative signaling molecules, respectively, of the Wnt/β-catenin signaling pathway. Signaling molecules of the Wnt signaling pathway include, but are not limited to, β-catenin, tumor suppressor gene product adenomatous polyposis coll (APC), axin, glycogen synthase kinase (GSK)-3β, TCF/LEF transcription factors, crescent, groucho, CBP, frizzled receptor, frizzled related proteins, LRP, LRP5, LRP6, kremin, Dvl/Dsh (disheveled), dickkopf, GSK-3 binding protein (GBP), FRAT/GBP, and any of the Wnt signaling pathway factors listed at http://www.stanford.edu/˜musse/pathways/cell2.html.

“β-catenin signal-promoting agent” refers to agonists or antagonists of positive or negative signaling molecules, respectively, of β-catenin signaling, e.g., any agent that activates β-catenin signaling through inhibition of GSK-3 in the presence or absence of Wnt signaling. For example, activation of β-catenin signaling in the absence of Wnt signaling can occur by activation of integrin linked kinase, activation of p53 leading to activation of Siah1, or activation of FGF signaling. “β-catenin signal-promoting agent” further refers to any signaling molecule that activates β-catenin target genes and is achieved by inhibition of GSK-3 that can have therapeutic potential. “β-catenin signal-promoting agent” further refers to any signaling molecule that activates β-catenin target genes independent of GSK-3 that can have therapeutic potential. Activation of β-catenin target genes without inhibiting GSK-3 can be achieved by inhibition (for example, by drug therapy, RNAi therapy or gene therapy) of any inhibitor of β-catenin function, including, but not limited to, APC, Axin, Chibby, ICAT, Groucho, CtBP.

“Wnt signal- or β-catenin signal-promoting agent or agonist” refers to one or more of the following: a nucleic acid comprising a nucleotide sequence that encodes a Wnt polypeptide, a polypeptide comprising an amino acid sequence of a Wnt polypeptide, a nucleic acid comprising a nucleotide sequence that encodes an activated Wnt receptor, a polypeptide comprising an amino acid sequence of an activated Wnt receptor, a small organic molecule that promotes Wnt/β-catenin signaling, a small organic molecule that inhibits the expression or activity of a Wnt or β-catenin, an antisense oligonucleotide that inhibits expression of a Wnt, β-catenin a ribozyme that inhibits expression of a Wnt or β-catenin antagonist, an RNAi construct, siRNA, or shRNA that inhibits expression of a Wnt or β-catenin antagonist, an antibody that binds to and inhibits the activity of a Wnt or β-catenin antagonist, a nucleic acid comprising a nucleotide sequence that encodes a β-catenin polypeptide, a polypeptide comprising an amino acid sequence of a β-catenin polypeptide, a nucleic acid comprising a nucleotide sequence that encodes a Lef-1 polypeptide, a polypeptide comprising an amino acid sequence of a Lef-1 polypeptide.

“Wnt/β-catenin signal-promoting agent or agonist” refers to one or more of the following: a nucleic acid comprising a nucleotide sequence that encodes a dominant negative GSK-3, GSK3α, or GSK3β polypeptide, a polypeptide comprising an amino acid sequence of a dominant negative GSK-3, GSK3α, or GSK3β polypeptide, a small organic molecule that binds to and inhibits the expression or activity of GSK-3, GSK3α, or GSK3β, an RNAi construct, siRNA, or shRNA that binds to and inhibits the expression and/or activity of GSK-3, GSK3a, or GSK3β, an antisense oligonucleotide that binds to and inhibits the expression of GSK-3, GSK3α, or GSK3β, an antibody that binds to and inhibits the expression and/or activity of GSK-3, GSK3α, or GSK3β, a ribozyme that binds to and inhibits the expression of GSK-3, GSK3a, or GSK3β, and any GSK-3-independent reagent that activates β-catenin target genes similar in effect to GSK-3 inhibition. Further examples of Wnt/β-catenin signal-promoting agent or agonist can be an inhibitor of Axin2, a dominant negative Axin2 polypeptide, a polypeptide comprising an amino acid sequence of a dominant negative Axin2 polypeptide, a small organic molecule that binds to and inhibits the expression or activity of Axin2, an RNAi construct, siRNA, or shRNA that binds to and inhibits the expression and/or activity of Axin2, an antisense oligonucleotide that binds to and inhibits the expression of Axin2, an antibody that binds to and inhibits the expression and/or activity of Axin2, a ribozyme that binds to and inhibits the expression of Axin2.

Exemplary Wnt/β-catenin pathway signal-promoting agent or agonist(s) include, but are not limited to, LiCl or other GSK-3 inhibitors, as exemplified in U.S. Pat. Nos. 6,057,117 and 6,608,063; and U.S. applications 2004/0092535 and 2004/0209878; ATP-competitive, selective GSK-3 inhibitors CHIR-911 and CHIR-837 (also referred to as CT-99021 and CT-98023 respectively). Chiron Corporation (Emeryville, Calif.). These inhibitors were purified to >95% by high-performance liquid chromatography. CHIR-911 was formulated in 10% captisol solution for administration in vivo by intraperitoneal injection, with a half-maximal effective concentration [EC₅₀] of 766 nM and >10,000 fold selectivity for GSK-3. Ring et al., Diabetes 52: 588-595, 2003. CHIR-837 was formulated in DMSO for in vitro use, with an EC₅₀ of 375 nM and >5,000 fold selectivity for GSK-3 Cline et al., Diabetes 51: 2903-2910, 2002 each incorporated herein by reference in their entirety.

“β-catenin-independent signaling” refers to wnt-mediated signaling that does not activate β-catenin-mediated transcription, for example, signaling via wnt5b wherein wnt5b antagonizes Wnt/β-catenin signaling pathway and inhibits regeneration. It is possible that β-catenin-independent signaling pathways also inhibit regeneration without impairing Wnt/β-catenin signaling. The β-catenin-dependent and β-catenin-independent Wnt signaling pathways have opposing roles in zebrafish fin regeneration. Wnts can act through the Wnt/β-catenin pathway (wnt10a) and through β-catenin-independent pathways (wnt5a, wnt5b/pipetail). For example, overexpression of Wnt8 in hsWnt8GFP transgenic fish induces the Wnt/β-catenin target gene axin2 in regenerating fins six hours after heat-shock at 3 dpa, while overexpression of Wnt5b in hsWnt5bGFP transgenic fish represses axin2 expression.

Inhibitors of β-catenin-independent signaling” refers to compounds that inhibit signaling via wnt5b. Inhibitors of β-catenin-independent signaling include, but are not limited to, one or more of the following compounds that inhibit signaling via wnt5b: a nucleic acid comprising a nucleotide sequence that encodes an inactive Wnt5b receptor, a polypeptide comprising an amino acid sequence of an inactive Wnt receptor, a small chemical molecule or a small organic molecule that inhibits the expression or activity of a Wnt5b, an antisense oligonucleotide that inhibits expression of a Wnt5b, a ribozyme that inhibits expression of a Wnt5b, an RNAi construct, siRNA, or shRNA that inhibits expression of a Wnt5b, or an antibody that binds to and inhibits the activity of Wnt5b.

The present invention is directed to methods for increasing non-terminally differentiated cells in vivo, e.g., progenitor cells or stem cells, by activating the Wnt/β-catenin signaling pathway in a progenitor/stem cell to enhance or promote regeneration by increasing activity or proliferation of progenitor/stem cells. Activation of the Wnt/β-catenin signaling pathway can promote regeneration by a variety of cellular mechanisms, for example, by activating or promoting progenitor/stem cell function, by promoting differentiation of progenitor/stem cell, or a combination thereof. “Precursor cells” or “progenitor/stem cell” shall mean any non-terminally differentiated cells. The present invention is also directed to methods for increasing non-terminally differentiated cells in vivo, e.g., progenitor cells or stem cells, by activating the Wnt/β-catenin signaling in the cells such that the differentiation of the progenitor/stem cell is inhibited without affecting the mitotic activity of the cells. Further, the progenitor/stem cells can be isolated from a cell population, if desired, before or after Wnt/β-catenin signaling pathway activation.

“Adult” refers to tissues and cells derived from or within an animal subject at any time after birth. “Embryonic” refers to tissues and cells derived from or within an animal subject at any time prior to birth.

Vertebrate subject refers to a vertebrate subject, e.g., human, primate, rats, mice, rabbit, cat, dog, avian, a reptilian subject, amphibian subject, osteichthyes subject, or chondrichthyes subject.

“Blood development” refers to hematopoiesis and vascular growth. “Vascular growth” refers to at least one of vasculogenesis and angiogenesis and includes formation of capillaries, arteries, veins or lymphatic vessels.

“Hematopoiesis” refers to the process of production of progenitor/stem cells from which many cell types are derived. “Hematopoietic stem cell” refers to a multipotential precursor from which all classes of blood cell are derived; in addition, mesenchymal progenitor/stem cells, mesodermal progenitor/stem cells, endothelial progenitor/stem cells, and ectodermal or neural progenitor/stem cells can be derived from hematopoietic stem cell. “Definitive blood cells” refers to blood cells of the fetal or adult organism. “Primitive blood cells” refers to a transient population of blood cells forming during blood development in the embryo.

“Progenitor cells” or “stem cells” refers to undifferentiated cells that are more restricted in their potential to give rise to differentiated cell types compared with a stem cell. The progenitor cell or stem cell includes, but is not limited to, hematopoietic progenitor/stem cell, mesenchymal progenitor/stem cells, mesodermal progenitor/stem cells, epithelial progenitor/stem cell, kidney progenitor/stem cell, neural progenitor/stem cell, skin progenitor/stem cell, osteoblast progenitor/stem cell, chondrocyte progenitor/stem cell, liver progenitor/stem cell, or muscle progenitor/stem cell.

“Differentiated cell population” refers to cells or tissues in a committed cell lineage. The cell or tissue includes, but is not limited to, bone, chondrocytes/cartilage, muscle, skeletal muscle, cardiac muscle, pancreatic cells, endothelial cells, vascular endothelial cells, adipose cells, liver, skin, connective tissue, hematopoietic stem cells, neonatal cells, umbilical cord blood cells, fetal liver cells, adult cells, bone marrow cells, peripheral blood cells, erythroid cells, granulocyte cells, macrophage cells, granulocyte-macrophage cells, B cells, T cells, multipotent mixed lineage colony types, embryonic stem cells, mesenchymal progenitor/stem cells, mesodermal progenitor/stem cells, neural progenitor/stem cells, or nerve cells.

“Cell or tissue regeneration” refers to the ability to restore lost or damaged tissues, organs, or limbs by an increase in stem cell, progenitor cell or differentiated cell populations in a vertebrate subject which lead to an increase in the number of differentiated tissue-specific cells in the vertebrate subject.

“Committed” refers to cells destined to differentiate along a specific lineage instead of retaining multipotency.

“Synergistic effect” refers to two or more compounds where the biological effect of the compounds together is more than the added biological effects of the compounds acting alone.

Embryonic stem cells (ESCs), for example, human embryonic stem cells (hESCs), can be used to derive progenitor/stem cells including but not limited to, hematopoietic progenitor/stem cell, mesenchymal progenitor/stem cell, mesodermal progenitor/stem cell, endothelial progenitor/stem cell, or ectodermal or neural progenitor/stem cell. A subpopulation of primitive endothelial-like cells have been identified that are derived from human embryonic stem cells (hESCs) that express PECAM-1, Flk-1, and VE-cadherin, but not CD45 (CD45negPFV cells), and that are uniquely responsible for endothelial and hematopoietic development. Human hematopoiesis and endothelial maturation can originate from a subset of embryonic endothelial cells that possesses hemangioblastic properties.

A method for increasing stem cell or progenitor cell population by means of activating or promoting progenitor/stem cell function, by promoting differentiation of progenitor/stem cells, or a combination thereof, is provided to repair or replace damaged tissue in a vertebrate subject, wherein the cell or tissue regeneration occurs in bone, chondrocytes/cartilage, muscle, skeletal muscle, cardiac muscle, pancreatic cells, endothelial cells, vascular endothelial cells, adipose cells, liver, skin, connective tissue, hematopoietic stem cells, neonatal cells, umbilical cord blood cells, fetal liver cells, adult cells, bone marrow cells, peripheral blood cells, erythroid cells, granulocyte cells, macrophage cells, granulocyte-macrophage cells, B cells, T cells, multipotent mixed lineage colony types, embryonic stem cells, mesenchymal progenitor/stem cells, mesodermal progenitor/stem cells, neural progenitor/stem cells, or nerve cells.

A method of treating a disease or conditions using methods of the present invention, include but are not limited to the following disease states: muscle degenerative disease, muscular dystrophy or myopathy, cardiomyopathy, cardiac ischemia, neurodegenerative diseases, Alzheimer's disease, taupathies, stroke, ischemia, liver degenerative diseases, liver cirrosis, chronic or acute hepatitis, bone density disease, or osteoprosis.

Other potential therapeutic activators of Wnt/β-catenin signaling include agents that have no obvious link to the β-catenin pathway, such as extracellular calcium, non-steroidal anti-inflammatory drugs, including exisulind, and the tyrosine kinase inhibitor STI571/Gleevac96. Conversely, activators of β-catenin signaling will probably be useful in treating osteoporosis and Alzheimer's disease, and might include activators of LRP5 as well as inhibitors of GSK3. Moon et al., Nature Reviews Genetics 5: 689-699, 2004, each incorporated by reference in their entirety.

A method of treating a degenerative muscle disease is provided wherein the disease is a muscular dystrophy or myopathy. Muscular dystrophy (MD) refers to a group of genetic diseases characterized by progressive weakness and degeneration of the skeletal muscles which control movement. There are many forms of muscular dystrophy, some noticeable at birth (congenital muscular dystrophy), others in adolescence (Becker MD). The 3 most common types are Duchenne, facioscapulohumeral, and myotonic. These three types differ in terms of pattern of inheritance, age of onset, rate of progression, and distribution of weakness. Duchenne muscular dystrophy primarily affects boys and is the result of mutations in the gene that regulates dystrophin—a protein involved in maintaining the integrity of muscle fiber. Onset is between 3-5 years and progresses rapidly. Facioscapulohumeral muscular dystrophy appears in adolescence and causes progressive weakness in facial muscles and certain muscles in the arms and legs. It progresses slowly and can vary in symptoms from mild to disabling. Myotonic muscular dystrophy varies in the age of onset and is characterized by myotonia (prolonged muscle spasm) in the fingers and facial muscles; a floppy-footed, high-stepping gait; cataracts; cardiac abnormalities; and endocrine disturbances.

“Myopathy” refers to neuromuscular disorders in which the primary symptom is muscle weakness due to dysfunction of muscle fiber. Other symptoms of myopathy can include include muscle cramps, stiffness, and spasm. Myopathies can be inherited (such as the muscular dystrophies) or acquired (such as common muscle cramps). Congenital myopathy is characterized by developmental delays in motor skills; skeletal and facial abnormalities are occasionally evident at birth. Muscular dystrophy is characterized by progressive weakness in voluntary muscles; sometimes evident at birth. Mitochondrial myopathy is caused by genetic abnormalities in mitochondria, cellular structures that control energy; include Kearns-Sayre syndrome, MELAS and MERRF. Glycogen storage diseases of muscle is caused by mutations in genes controlling enzymes that metabolize glycogen and glucose (blood sugar); include Pompe's, Andersen's and Cori's diseases. Myoglobinuria is caused by disorders in the metabolism of a fuel (myoglobin) necessary for muscle work; include McArdle, Tarui, and DiMauro diseases. Dermatomyositis is an inflammatory myopathy of skin and muscle. Myositis ossificans is characterized by bone growing in muscle tissue. Familial periodic paralysis is characterized by episodes of weakness in the arms and legs. Polymyositis, inclusion body myositis, and related myopathies are inflammatory myopathies of skeletal muscle. Neuromyotonia is characterized by alternating episodes of twitching and stiffness, and stiff-man syndrome is characterized by episodes of rigidity and reflex spasms. Common muscle cramps and stiffness, and tetany is characterized by prolonged spasms of the arms and legs

Cardiomyopathy refers to a disease of the myocardium associated with ventricular dysfunction as defined by the World Health Organization. Dilated cardiomyopathy is characterized by dilatation and impaired contractility of the left (or right) ventricle. Presentation is usually with heart failure. Arrhythmia, thromboembolism, and sudden death are common. Hypertrophic cardiomyopathy is characterized by left (or right) ventricular hypertrophy, which is usually asymmetric and involves the interventricular septum. Typically, left ventricular volume is reduced. Systolic gradients are sometimes present. Typical presentations include dyspnea, arrhythmia, and sudden death. Restrictive cardiomyopathy is characterized by restrictive filling of the left (or right) ventricle with normal or near normal ventricular contractility and wall thickness. Presentations are usually with heart failure. The cardiomyopathies are not the only causes of the heart failure syndrome. In western countries, coronary artery disease with resultant ischemic cardiomyopathy remains the primary cause of the heart failure syndrome.

A method of treating a neurodegenerative disease is provided wherein the disease is a central nervous system disorder or peripheral nervous system disorder. Central nervous system disorders encompass numerous afflictions such as neurodegenerative diseases (e.g., Alzheimer's disease and Parkinson's disease), acute brain injury (e.g., stroke, head injury, cerebral palsy) and a large number of CNS dysfunctions (e.g., depression, epilepsy, and schizophrenia). These diseases, which include Alzheimer's disease, multiple sclerosis (MS), Huntington's disease, amyotrophic lateral sclerosis, and Parkinson's disease, have been linked to the degeneration of neural cells in particular locations of the CNS, leading to the inability of these cells or the brain region to carry out their intended function.

“Activators,” “inhibitors,” and “modulators” of Wnt/β-catenin signaling are used to refer to activating, or modulating molecules, respectively, identified using in vitro and in vivo assays for Wnt/β-catenin signal-promoting agents, e.g., ligands, agonists, antagonists, and their homologs and mimetics. “Modulator” includes inhibitors and activators. Inhibitors are agents that, e.g., bind to, partially or totally block stimulation, decrease, prevent, delay activation, inactivate, desensitize, or down regulate the activity of Wnt/β-catenin signaling, e.g., antagonists. Activators are agents that, e.g., bind to, stimulate, increase, open, activate, facilitate, enhance activation, sensitize or up regulate the activity of Wnt/β-catenin signaling, e.g., agonists. Modulators include agents that, e.g., alter the interaction of Wnt and β-catenin with: proteins that bind activators or inhibitors, receptors, including proteins, peptides, lipids, carbohydrates, polysaccharides, or combinations of the above, e.g., lipoproteins, glycoproteins, and the like. Modulators include genetically modified versions of naturally-occurring Wnt or β-catenin ligands, e.g., Wnt or β-catenin polypeptides with altered activity, as well as naturally occurring and synthetic ligands, antagonists, agonists, small chemical molecules and the like. Such assays for inhibitors and activators include, e.g., applying putative modulator compounds to a cell expressing Wnt or β-catenin and then determining the functional effects on Wnt/β-catenin signaling as described herein. Samples or assays comprising Wnt/β-catenin signaling that are treated with a potential activator, inhibitor, or modulator are compared to control samples without the inhibitor, activator, or modulator to examine the extent of inhibition. Control samples (untreated with activators, inhibitors, or modulators) can be assigned a relative Wnt or β-catenin activity value of 100%. Inhibition of Wnt/β-catenin signaling is achieved when the Wnt or β-catenin activity value relative to the control is about 80%, optionally 50% or 25-0%. Activation of Wnt/β-catenin signaling is achieved when the Wnt or β-catenin activity value relative to the control is 110%, optionally 150%, optionally 200-500%, or 1000-3000% higher. Activation of Wnt/β-catenin signaling can be measured in a cellular assay for increasing hematopoietic progenitor/stem cells in vivo in a vertebrate subject or increasing mesenchymal progenitor/stem cells, mesodermal progenitor/stem cells, neural progenitor/stem cells, muscle progenitor/stem cells or stem cells in vivo in a vertebrate subject, as described herein.

“Agonist” is used in the broadest sense and includes any molecule that mimics or enhances a biological activity of Wnt or β-catenin polypeptides or Wnt/β-catenin signaling. Suitable agonist molecules specifically include agonist antibodies or antibody fragments, fragments or amino acid sequence variants of native Wnt or β-catenin polypeptides, peptides, antisense oligonucleotides, small organic molecules, and the like. Methods for identifying agonists of Wnt or β-catenin polypeptides can comprise contacting a Wnt or β-catenin polypeptide with a candidate agonist molecule and measuring a detectable change in one or more biological activities normally associated with the Wnt/β-catenin signaling e.g., Wnt binding to the Frizzled receptor, or intracellular accumulation of β-catenin.

“Antagonist” is used in the broadest sense, and includes any molecule that partially or fully blocks, inhibits, or neutralizes an inhibitor of a biological activity of a Wnt or catenin polypeptide, or Wnt/β-catenin signaling. Suitable antagonist molecules specifically include antagonist antibodies or antibody fragments, fragments or amino acid sequence variants of native polypeptides, peptides, antisense oligonucleotides, small organic molecules, and the like. Methods for identifying antagonists of an inhibitor of a biological activity of a Wnt or β-catenin polypeptide, or of Wnt- or β-catenin-signaling can comprise contacting Wnt or β-catenin polypeptides, or Wnt- or β-catenin-signaling polypeptides with a candidate antagonist molecule and measuring a detectable change in one or more biological activities normally associated with Wnt/β-catenin signaling.

“Signaling in cells” refers to the interaction of a ligand, such as an endogenous or exogenous ligand, e.g., Wnt signal-promoting or β-catenin signal-promoting agents, with receptors, such as Frizzled receptor, resulting in cell signaling to produce a response, for example, increasing expression of β-catenin target genes resulting in increased hematopoietic progenitor/stem cells in vivo in a vertebrate subject.

“Endogenous” refers a protein, nucleic acid, lipid or other component produced within the body or within cells or organs of the body of a vertebrate subject or originating within cells or organs of the body of a vertebrate subject.

“Exogenous” refers a protein, nucleic acid, lipid, or other component originating outside the cells, organs and/or body of a vertebrate subject.

“Test compound” refers to a nucleic acid, DNA, RNA, protein, polypeptide, or small chemical entity that is determined to effect an increase or decrease in a gene expression as a result of signaling through the Wnt/β-catenin signaling pathways. The test compound can be an antisense RNA, ribozyme, polypeptide, or small molecular chemical entity. “Test compound” can be any small chemical compound, or a biological entity, such as a protein, sugar, nucleic acid or lipid. Typically, test compounds will be small chemical molecules and polypeptides. A “test compound specific for Wnt/β-catenin signaling” is determined to be a modulator of Wnt signaling or β-catenin signaling, for example, resulting in Wnt binding to the Frizzled receptor, or intracellular accumulation of β-catenin.

“Cell-based assays” include Wnt/β-catenin signaling assays, for example, radioligand or fluorescent ligand binding assays for Wnt or β-catenin to cells, plasma membranes, detergent-solubilized plasma membrane proteins, immobilized collagen (Alberdi, J Biol Chem. 274:31605-12, 1999; Meyer et al., 2002); Wnt or β-catenin-affinity column chromatography (Alberdi, J Biol Chem. 274:31605-12, 1999; Aymerich et al., Invest Opthalmol Vis Sci. 42:3287-93, 2001); Wnt or β-catenin blot using a radio- or fluorosceinated-ligand (Aymerich et al., Invest Opthalmol Vis Sci. 42:3287-93, 2001; Meyer et al., 2002); Size-exclusion ultrafiltration (Alberdi et al., 1998, Biochem.; Meyer et al., 2002); or ELISA. Exemplary Wnt/β-catenin signaling activity assays of the present invention are: Assays for Wnt-regulated target gene Axin2 quantified by real-time PCR; Yan et al., Proc Natl Acad Sci USA 98:14973-8, 2001; Jho et al., Mol Cell Biol 22:1172-83, 2002. Wnt-regulated target gene CyclinD1 quantified by real-time PCR; Issack and Ziff, Cell Growth Differ 9:837-45, 1998. Notch regulated target gene, Hes1, quantified by real-time PCR; Jarriault et al., Nature 377:355-8, 1995. Hedgehog regulated target genes, Gli3 and Patched1 (Ptc1), quantified by real-time PCR. Marigo et al., 180 1:1996; Marigo and Tabin, Proc Natl Acad Sci USA 93:9346-9351, 1996, each incorporated by reference in their entirety. Further cell based assays include but are not limited to, luciferase, green fluorescent protein (GFP), or β-galactosidase reporter screens for β-catenin responsive genes, for example using TOPFLASH reporter. See, for example, Veeman M. et al., Current Biology, 13: 680-685, 2003; Veeman M. et al., Dev Cell. 5: 367-377, 2003, each incorporated by reference in their entirety.

“Signaling responsiveness” or “effective to activate signaling” or “stimulating a cell-based assay system” refers to the ability of Wnt/β-catenin signal-promoting agent to enhance tissue regeneration, to increase hematopoietic progenitor/stem cells, stem cells, mesenchymal progenitor/stem cells, mesodermal progenitor/stem cells muscle progenitor cells, or neural progenitor cells in vivo in a vertebrate subject, or treating a degenerative muscle disease or neurodegenerative disease in a vertebrate subject.

“Detecting an effect” refers to an effect measured in a cell-based assay system. For example, the effect detected can be Wnt/β-catenin signaling in an assay system, for example, Wnt or β-catenin cellular assay, Frizzled receptor binding assay, Axin2 assay, or CyclinD1 assay, or β-catenin-responsive gene reporter assay. See, for example, Veeman M. et al., Current Biology, 13: 680-685, 2003; Veeman M. et al., Dev Cell. 5: 367-377, 2003, each incorporated by reference in their entirety.

“Biological activity” and “biologically active” with regard to Wnt/β-catenin pathway agonist(s) and/or one or more inhibitor(s) of β-catenin-independent signaling of the present invention refer to the ability of the ligand molecule to specifically bind to and signal through a native or recombinant Wnt or β-catenin, or to block the ability of an inhibitor of native or recombinant Wnt or β-catenin polypeptides to participate in signal transduction. Thus, the (native and variant) Wnt/β-catenin pathway agonist(s) and/or one or more inhibitor(s) of β-catenin-independent signaling of the present invention include agonists of a native or recombinant Wnt or β-catenin polypeptides and receptors or ligands thereof. Preferred biological activities of the Wnt/β-catenin pathway agonist(s) and/or one or more inhibitor(s) of β-catenin-independent signaling of the present invention include the ability to induce or inhibit, for example, enhancing tissue regeneration, or increasing hematopoietic progenitor/stem cells, stem cells, muscle progenitor cells, or neural progenitor cells in vivo in a vertebrate subject or treating a degenerative muscle disease or neurodegenerative disease in a vertebrate subject. Accordingly, the administration of the compounds or agents of the present invention can prevent or delay, to alleviate, or to arrest or inhibit development of the symptoms or conditions associated with a degenerative muscle disease or neurodegenerative disease in a vertebrate subject.

“Signal transduction pathway” or “signal transduction event” refers to at least one biochemical reaction, but more commonly a series of biochemical reactions, which result from interaction of a cell with a stimulatory compound or agent. Thus, the interaction of a stimulatory compound with a cell generates a “signal” that is transmitted through the signal transduction pathway, ultimately resulting in a cellular response, e.g., a tissue regeneration response as described above.

“High affinity” for a ligand refers to an equilibrium association constant (Ka) of at least about 10³M⁻¹, at least about 10⁴ M⁻¹, at least about 10⁵ M⁻¹, at least about 10⁶ M⁻¹, at least about 10⁷ M⁻¹, at least about 10⁸ M⁻¹, at least about 10⁹ M⁻¹, at least about 10¹⁰ M⁻¹, at least about 10¹¹ M⁻¹, or at least about 10¹² M⁻¹ or greater, e.g., up to 10¹³ M⁻¹ or 10¹⁴ M⁻¹ or greater. However, “high affinity” binding can vary for other ligands.

“Kₐ”, as used herein, is intended to refer to the equilibrium association constant of a particular ligand-receptor interaction, e.g., antibody-antigen interaction. This constant has units of 1/M.

“K_{d}”, as used herein, is intended to refer to the equilibrium dissociation constant of a particular ligand-receptor interaction. This constant has units of M.

“kₐ”, as used herein, is intended to refer to the kinetic association constant of a particular ligand-receptor interaction. This constant has units of 1/Ms.

“k_{d}”, as used herein, is intended to refer to the kinetic dissociation constant of a particular ligand-receptor interaction. This constant has units of 1/s.

“Particular ligand-receptor interactions” refers to the experimental conditions under which the equilibrium and kinetic constants are measured.

“Isotype” refers to the antibody class that is encoded by heavy chain constant region genes. Heavy chains are classified as gamma, mu, alpha, delta, or epsilon, and define the antibody's isotype as IgG, IgM, IgA, IgD and IgE, respectively. Additional structural variations characterize distinct subtypes of IgG (e.g., IgG₁, IgG₂, IgG₃ and IgG₄) and IgA (e.g., IgA₁ and IgA₂)

The ability of a molecule to bind to Wnt or β-catenin can be determined, for example, by the ability of the putative ligand to modulate Wnt binding to the Frizzled receptor, by measuring intracellular accumulation of β-catenin, Axin2 assay, or CyclinD1 assay. Specificity of binding can be determined by comparing binding in the presence or absence of the putative ligand.

“Control sequences” or “regulatory sequences” refers to DNA sequences necessary for the expression of an operably linked coding sequence in a particular host organism. The control sequences that are suitable for prokaryotes, for example, include a promoter, optionally an operator sequence, a ribosome binding site, and possibly, other as yet poorly understood sequences. Eukaryotic cells are known to utilize promoters, polyadenylation signals, and enhancers.

“Vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal vertebrate vectors). Other vectors (e.g., non-episomal vertebrate vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

A “label” is a composition detectable by spectroscopic, photochemical, biochemical, immunochemical, or chemical means. For example, useful labels include ³²P, fluorescent dyes, electron-dense reagents, enzymes (e.g., as commonly used in an ELISA), biotin, digoxigenin, or haptens and proteins for which antisera or monoclonal antibodies are available (e.g., the polypeptides of the invention can be made detectable, e.g., by incorporating a radiolabel into the peptide, and used to detect antibodies specifically reactive with the peptide).

“Sorting” in the context of cells as used herein to refers to both physical sorting of the cells, as can be accomplished using, e.g., a fluorescence activated cell sorter (FACS), as well as to analysis of cells based on expression of cell surface markers, e.g., FACS analysis in the absence of sorting.

“Cell,” “cell line,” and “cell culture” are used interchangeably and all such designations include progeny. Thus, the words “transformants” and “transformed cells” include the primary subject cell and cultures derived therefrom without regard for the number of transfers. It is also understood that all progeny cannot be precisely identical in DNA content, due to deliberate or inadvertent mutations. Mutant progeny that have the same function or biological activity as screened for in the originally transformed cell are included. Where distinct designations are intended, it will be clear from the context.

“Receptor” denotes a cell-associated protein, for example Frizzled receptor, that binds to a bioactive molecule termed a “ligand.” This interaction mediates the effect of the ligand on the cell. Receptors can be membrane bound, cytosolic or nuclear; monomeric (e.g., Frizzled receptor, thyroid stimulating hormone receptor, beta-adrenergic receptor) or multimeric (e.g., PDGF receptor, growth hormone receptor, IL-3 receptor, GM-CSF receptor, G-CSF receptor, erythropoietin receptor and IL-6 receptor). Membrane-bound receptors, for example Frizzled receptor, are characterized by a multi-domain structure comprising an extracellular ligand-binding domain and an intracellular effector domain that is typically involved in signal transduction. In certain membrane-bound receptors, the extracellular ligand-binding domain and the intracellular effector domain are located in separate polypeptides that comprise the complete functional receptor.

In general, the binding of ligand to receptor results in a conformational change in the receptor that causes an interaction between the effector domain and other molecule(s) in the cell, which in turn leads to an alteration in the metabolism of the cell. Metabolic events that are often linked to receptor-ligand interactions include gene transcription, phosphorylation, dephosphorylation, increases in cyclic AMP production, mobilization of cellular calcium, mobilization of membrane lipids, cell adhesion, hydrolysis of inositol lipids and hydrolysis of phospholipids.

“Treatment” or “treating” refers to any indicia of success in the treatment or amelioration of an injury, pathology or condition, including any objective or subjective parameter such as abatement; remission; diminishing of symptoms or making the injury, pathology, or condition more tolerable to the patient; slowing in the rate of degeneration or decline; making the final point of degeneration less debilitating; or improving a subject's physical or mental well-being. The treatment or amelioration of symptoms can be based on objective or subjective parameters; including the results of a physical examination. Accordingly, “treatment” or “treating” includes the administration of the compounds or agents of the present invention to enhance a tissue regeneration response, or treat muscle degenerative disease, muscular dystrophy or myopathy, cardiomyopathy, cardiac ischemia, neurodegenerative diseases, Alzheimer's disease, taupathies, stroke, ischemia, liver degenerative diseases, liver cirrosis, chronic or acute hepatitis, skin disease, bone density disease, or osteoprosis. Accordingly, “treatment” or “treating” includes the administration of the compounds or agents of the present invention to prevent or delay, to alleviate, or to arrest or inhibit development of the symptoms or conditions associated with a degenerative muscle disease, neurodegenerative disease, liver degenerative disease, bone density disease, or skin disease, or other disorders. “Therapeutic effect” refers to the reduction, elimination, or prevention of the disease, symptoms of the disease, or side effects of the disease in the subject.

“Concomitant administration” of a known drug with a compound of the present invention means administration of the drug and the compound at such time that both the known drug and the compound will have a therapeutic effect or diagnostic effect. Such concomitant administration can involve concurrent (i.e. at the same time), prior, or subsequent administration of the drug with respect to the administration of a compound of the present invention. A person of ordinary skill in the art, would have no difficulty determining the appropriate timing, sequence and dosages of administration for particular drugs and compounds of the present invention.

“Subject”, “vertebrate subject” or “patient” refers to any vertebrate patient or subject to which the compositions of the invention can be administered. “Mammal” or “vertebrate” refers to human patients and non-human primates, as well as experimental animals such as rabbits, rats, and mice, and other animals. In an exemplary embodiment, of the present invention, to identify subject patients for treatment according to the methods of the invention, accepted screening methods are employed to determine risk factors associated with a targeted or suspected disease or condition or to determine the status of an existing disease or condition in a subject. These screening methods include, for example, conventional work-ups to determine risk factors that can be associated with the targeted or suspected disease or condition. These and other routine methods allow the clinician to select patients in need of therapy using the methods and formulations of the invention.

By “solid phase” is meant a non-aqueous matrix to which a reagent of interest (e.g., Wnt, β-catenin, or Frizzled receptor, or an antibody thereto) can adhere. Examples of solid phases encompassed herein include those formed partially or entirely of glass (e.g., controlled pore glass), polysaccharides (e.g., agarose), polyacrylamides, polystyrene, polyvinyl alcohol and silicones. In certain embodiments, depending on the context, the solid phase can comprise the well of an assay plate; in others it is a purification column (e.g., an affinity chromatography column). This term also includes a discontinuous solid phase of discrete particles, such as those described in U.S. Pat. No. 4,275,149.

“Specifically (or selectively) binds” to an antibody refers to a binding reaction that is determinative of the presence of the protein in a heterogeneous population of proteins and other biologics. Thus, under designated immunoassay conditions, the specified antibodies bind to a particular protein at least two times the background and do not substantially bind in a significant amount to other proteins present in the sample.

“Specifically bind(s)” or “bind(s) specifically” when referring to a peptide refers to a peptide molecule which has intermediate or high binding affinity, exclusively or predominately, to a target molecule. The phrase “specifically binds to” refers to a binding reaction which is determinative of the presence of a target protein in the presence of a heterogeneous population of proteins and other biologics. Thus, under designated assay conditions, the specified binding moieties bind preferentially to a particular target protein and do not bind in a significant amount to other components present in a test sample. Specific binding to a target protein under such conditions can require a binding moiety that is selected for its specificity for a particular target antigen. A variety of assay formats can be used to select ligands that are specifically reactive with a particular protein. For example, solid-phase ELISA immunoassays, immunoprecipitation, Biacore and Western blot are used to identify peptides that specifically affect Wnt signaling or β-catenin signaling. Typically a specific or selective reaction will be at least twice background signal or noise and more typically more than 10 times background. Specific binding between a monovalent peptide and Wnt signaling, β-catenin signaling, or Frizzled receptor proteins means a binding affinity of at least 10³ M⁻¹, and preferably 10⁵, 10⁶, 10⁷, 10⁸, 10⁹ or 10¹⁰ M⁻¹. The binding affinity of Wnt to the Frizzled receptor is between about 10⁶ M⁻¹ to about 10¹⁰ M⁻¹.

The present invention is based on the discovery that in vivo administration of one or more Wnt/β-catenin signal-promoting agents, e.g., Wnt8, or an analog or mimetic thereof, or an inhibitor of β-catenin-independent signaling increases stem cell, progenitor cell, or differentiated cell population in the vertebrate subject in vivo. In the present invention, the role of inhibitors of β-catenin-independent signaling have been investigated in the regulation of cell or tissue regeneration in a vertebrate subject, for example, inhibitors of Wnt5a or Wnt5b. The findings demonstrate that Wnt/β-catenin signal-promoting agents, e.g., Wnt8, or an analog or mimetic thereof, and inhibitors of β-catenin-independent signaling, e.g., inhibitors of Wnt5a or Wnt5b.augment cell or tissue regeneration in vivo and modulates Wnt, targets specifically in stem cell, progenitor cell, or differentiated cell populations, thereby providing a potent and unique approach to directly enhance cell or tissue regeneration in vivo.

This invention relies on routine techniques in the field of recombinant genetics. Basic texts disclosing the general methods of use in this invention include Sambrook et al., Molecular Cloning, A Laboratory Manual, 2nd ed., 1989; Kriegler, Gene Transfer and Expression: A Laboratory Manual, 1990; and Ausubel et al., eds., Current Protocols in Molecular Biology, 1994.

Wnt or β-catenin nucleic acids, polymorphic variants, orthologs, and alleles that are substantially identical to sequences provided herein can be isolated using Wnt or β-catenin, nucleic acid probes and oligonucleotides under stringent hybridization conditions, by screening libraries. Alternatively, expression libraries can be used to clone Wnt or β-catenin receptor protein, polymorphic variants, orthologs, and alleles by detecting expressed homologs immunologically with antisera or purified antibodies made against human Wnt or β-catenin portions thereof.

### Identification of Compounds for Treatment and Prophylaxis of Disease

(A) Identification of Bioactive Agents

Identifying bioactive agents that modulate Wnt/β-catenin signaling, the information is used in a wide variety of ways. In one method, one of several cellular assays, e.g., Wnt/β-catenin signaling assay, can be used in conjunction with high throughput screening techniques, to allow monitoring for antagonists or agonists of Wnt/β-catenin signaling after treatment with a candidate agent, Zlokarnik, et al., Science 279:84-8, 1998; and Heid et al., Genome Res. 6:986, 1996; each incorporated herein by reference in their entirety. In one method, the candidate agents are added to cells.

“Candidate bioactive agent” or “drug candidate” or grammatical equivalents as used herein describes any molecule, e.g., protein, oligopeptide, small organic molecule, polysaccharide, polynucleotide, to be tested for bioactive agents that are capable of directly or indirectly altering the activity of Wnt/β-catenin signaling. In one methods, the bioactive agents modulate Wnt/β-catenin signaling. In a further embodiment of the method, the candidate agents induce an antagonist or agonist effect in a Wnt/β-catenin signaling assay, as further described below. Generally a plurality of assay mixtures are run in parallel with different agent concentrations to obtain a differential response to the various concentrations. Typically, one of these concentrations serves as a negative control, i.e., at zero concentration or below the level of detection.

Candidate agents encompass numerous chemical classes, though typically they are organic molecules, e.g., small organic compounds having a molecular weight of more than 100 and less than about 2,500 daltons. Candidate agents comprise functional groups necessary for structural interaction with proteins, particularly hydrogen bonding, and typically include at least an amine, carbonyl, hydroxyl or carboxyl group, for example, at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof. In a further embodiment, candidate agents are peptides.

Candidate agents are obtained from a wide variety of sources including libraries of synthetic or natural compounds. For example, numerous means are available for random and directed synthesis of a wide variety of organic compounds and biomolecules, including expression of randomized oligonucleotides. Alternatively, libraries of natural compounds in the form of bacterial, fungal, plant and animal extracts are available or readily produced. Additionally, natural or synthetically produced libraries and compounds are readily modified through conventional chemical, physical and biochemical means. Known pharmacological agents can be subjected to directed or random chemical modifications, such as acylation, alkylation, esterification, amidification to produce structural analogs.

In some embodiments, the candidate bioactive agents are proteins. By “protein” herein is meant at least two covalently attached amino acids, which includes proteins, polypeptides, oligopeptides and peptides. The protein can be made up of naturally occurring amino acids and peptide bonds, or synthetic peptidomimetic structures. Thus “amino acid”, or “peptide residue”, as used herein means both naturally occurring and synthetic amino acids. For example, homo-phenylalanine, citrulline and noreleucine are considered amino acids for the purposes of the methods herein. “Amino acid” also includes imino acid residues such as proline and hydroxyproline. The side chains can be in either the (R) or the (S) configuration. In further embodiments, the amino acids are in the (S) or (L)-configuration. If non-naturally occurring side chains are used, non-amino acid substituents can be used, for example to prevent or retard in vivo degradations.

In one method, the candidate bioactive agents are naturally occurring proteins or fragments of naturally occurring proteins. Thus, for example, cellular extracts containing proteins, or random or directed digests of proteinaceous cellular extracts, can be used. In this way libraries of procaryotic and eucaryotic proteins can be made for screening using the methods herein. The libraries can be bacterial, fungal, viral, and vertebrate proteins, and human proteins.

In some methods, the candidate bioactive agents are peptides of from about 5 to about 30 amino acids, typically from about 5 to about 20 amino acids, and typically from about 7 to about 15 being. The peptides can be digests of naturally occurring proteins as is outlined above, random peptides, or “biased” random peptides. By “randomized” or grammatical equivalents herein is meant that each nucleic acid and peptide consists of essentially random nucleotides and amino acids, respectively. Since generally these random peptides (or nucleic acids, discussed below) are chemically synthesized, they can incorporate any nucleotide or amino acid at any position. The synthetic process can be designed to generate randomized proteins or nucleic acids, to allow the formation of all or most of the possible combinations over the length of the sequence, thus forming a library of randomized candidate bioactive proteinaceous agents.

In some methods, the library can be fully randomized, with no sequence preferences or constants at any position. In other methods, the library can be biased. Some positions within the sequence are either held constant, or are selected from a limited number of possibilities. For example, in some methods, the nucleotides or amino acid residues are randomized within a defined class, for example, of hydrophobic amino acids, hydrophilic residues, sterically biased (either small or large) residues, towards the creation of nucleic acid binding domains, the creation of cysteines, for cross-linking, prolines for SH-3 domains, serines, threonines, tyrosines or histidines for phosphorylation sites, or to purines. In other methods, the candidate bioactive agents are nucleic acids, as defined above.

As described above generally for proteins, nucleic acid candidate bioactive agents can be naturally occurring nucleic acids, random nucleic acids, or “biased” random nucleic acids. For example, digests of procaryotic or eucaryotic genomes can be used as is outlined above for proteins.

In some methods, the candidate bioactive agents are organic chemical moieties.

(B) Drug Screening Methods

Several different drug screening methods can be accomplished to identify drugs or bioactive agents that act as Wnt/β-catenin signal-promoting agents and/or one or more inhibitors of β-catenin-independent signaling. One such method is the screening of candidate agents that can act as agonists of Wnt/β-catenin signaling, thus generating the associated phenotype. Similarly, candidate agents that can act as an agonist to Wnt/β-catenin signaling, as shown herein, are expected to result in the immunostimulant phenotype, upon challenge with a pathogen. Thus, in some methods, candidate agents can be determined that mimic or alter Wnt/β-catenin signaling.

In other methods, screening can be done to alter the biological function of Wnt/β-catenin signaling. Again, having identified the importance of a Wnt binding to the Frizzled receptor, or intracellular accumulation of β-catenin, screening for agents that bind and/or modulate the biological activity of the Wnt/β-catenin signaling can be performed as outlined below.

Thus, screening of candidate agents that modulate Wnt/β-catenin signaling either at the level of gene expression or protein level can be accomplished.

In some methods, a candidate agent can be administered in any one of several cellular assays, e.g., Wnt/β-catenin signaling assay. By “administration” or “contacting” herein is meant that the candidate agent is added to the cells in such a manner as to allow the agent to act upon the cell, whether by uptake and intracellular action, or by action at the cell surface. In some embodiments, nucleic acid encoding a proteinaceous candidate agent (i.e., a peptide) can be put into a viral construct such as a retroviral construct and added to the cell, such that expression of the peptide agent is accomplished; see PCT US97/01019, incorporated herein by reference in its entirety.

Once the candidate agent has been administered to the cells, the cells can be washed if desired and are allowed to incubate under physiological conditions for some period of time. The cells are then harvested and a new gene expression profile is generated, as outlined herein.

For example, Wnt/β-catenin pathway agonist(s) and/or one or more inhibitor(s) of β-catenin-independent signaling can be screened for agents that produce tissue regenerating activity. A change in a binding assay or cellular assay indicates that the agent has an effect on Wnt/β-catenin signaling activity. In one method, a tissue regenerating activity is induced or maintained, before, during, and/or after stimulation with ligand. By defining such a signature for inhibiting or enhancing tissue regenerating activity or treatment for muscle degenerative disease or neurodegenerative disease screens for new drugs that mimic the tissue regenerating phenotype can be devised. With this approach, the drug target need not be known and need not be represented in the original expression screening platform, nor does the level of transcript for the target protein need to change. In some methods, the agent acts as an agonist or antagonist in one of several cellular or binding assays, e.g., Wnt/β-catenin signaling assay.

In some methods, screens can be done on individual genes and gene products. After having identified a cellular or binding assay as indicative of inhibition or enhancement of tissue regenerating activity, or treatment of a degenerative muscle disease or neurodegenerative disease, or screening of modulators of a cellular or binding assay Wnt/β-catenin signal-promoting agent or antagonist of β-catenin-independent signaling can be completed.

Thus, in some methods, screening for modulators of cellular or binding assay can be completed. This will be done as outlined above, but in general a few cellular or binding assay are evaluated. In some methods, screens are designed to first find candidate agents that can affect a cellular activity or binding assay, and then these agents can be used in other assays that evaluate the ability of the candidate agent to modulate Wnt/β-catenin signaling.

In general, purified or isolated gene product can be used for binding assays; that is, the gene products of Wnt/β-catenin signaling are made. Using the nucleic acids of the methods and compositions herein which encode Wnt or β-catenin polypeptides, or compounds of Wnt- or β-catenin-signaling, a variety of expression vectors can be made. The expression vectors can be either self-replicating extrachromosomal vectors or vectors which integrate into a host genome. Generally, these expression vectors include transcriptional and translational regulatory nucleic acid operably linked to the nucleic acid encoding a Wnt signaling or β-catenin protein. “Control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice. The transcriptional and translational regulatory nucleic acid will generally be appropriate to the host cell used to express Wnt/β-catenin signal-promoting protein; for example, transcriptional and translational regulatory nucleic acid sequences from Bacillus are used to express the protein in Bacillus. Numerous types of appropriate expression vectors, and suitable regulatory sequences are known in the art for a variety of host cells.

In general, the transcriptional and translational regulatory sequences can include, but are not limited to, promoter sequences, ribosomal binding sites, transcriptional start and stop sequences, translational start and stop sequences, and enhancer or activator sequences. In one method, the regulatory sequences include a promoter and transcriptional start and stop sequences.

Promoter sequences encode either constitutive or inducible promoters. The promoters can be either naturally occurring promoters or hybrid promoters. Hybrid promoters, which combine elements of more than one promoter, are also known in the art, and are useful in the methods herein.

In addition, the expression vector can comprise additional elements. For example, the expression vector can have two replication systems, thus allowing it to be maintained in two organisms, for example in vertebrate or insect cells for expression and in a procaryotic host for cloning and amplification. Furthermore, for integrating expression vectors, the expression vector contains at least one sequence homologous to the host cell genome, and typically two homologous sequences which flank the expression construct. The integrating vector can be directed to a specific locus in the host cell by selecting the appropriate homologous sequence for inclusion in the vector. Constructs for integrating vectors are well known in the art. Methods to effect homologous recombination are described in PCT US93/03868 and PCT US98/05223, each incorporated herein by reference in their entirety.

In some methods, the expression vector contains a selectable marker gene to allow the selection of transformed host cells. Selection genes are well known in the art and will vary with the host cell used.

One expression vector system is a retroviral vector system such as is generally described in PCT/US97/01019 and PCT/US97/01048, each incorporated herein by reference in their entirety.

The Wnt/β-catenin signal-promoting proteins of the present methods and compositions are produced by culturing a host cell transformed with an expression vector containing nucleic acid encoding Wnt/β-catenin signal-promoting polypeptide, under the appropriate conditions to induce or cause expression of the protein. The conditions appropriate for Wnt/β-catenin signal-promoting polypeptide expression will vary with the choice of the expression vector and the host cell, and will be easily ascertained by one skilled in the art through routine experimentation. For example, the use of constitutive promoters in the expression vector will require optimizing the growth and proliferation of the host cell, while the use of an inducible promoter requires the appropriate growth conditions for induction. In some methods, the timing of the harvest is important. For example, the baculoviral systems used in insect cell expression are lytic viruses, and thus harvest time selection can be crucial for product yield.

Appropriate host cells include yeast, bacteria, archebacteria, fungi, and insect and animal cells, including vertebrate cells. Of particular interest are Drosophila melanogaster cells, Saccharomyces cerevisiae and other yeasts, E. coli, Bacillus subtilis, SF9 cells, C129 cells, 293 cells, Neurospora, BHK, CHO, COS, and HeLa cells. In some methods, hematopoietic progenitor/stem cells or neural progenitor cells, muscle progenitor cells are host cells as provided herein, which for example, include non-recombinant cell lines, such as primary cell lines. In addition, purified primary stem cell, progenitor cell, or differentiated cell population for TNF assay derived from either transgenic or non-transgenic strains can also be used. The host cell can alternatively be an cell type known to have immunodeficiency disorder.

In one method, the Wnt/β-catenin signal-promoting proteins are expressed in mammalian cells. Mammalian expression systems can include retroviral systems. A mammalian promoter is any DNA sequence capable of binding mammalian RNA polymerase and initiating the downstream (3′) transcription of a coding sequence for Wnt/β-catenin signal-promoting protein into mRNA. A promoter will have a transcription initiating region, which is usually placed proximal to the 5′ end of the coding sequence, and a TATA box, using a located 25-30 base pairs upstream of the transcription initiation site. The TATA box is thought to direct RNA polymerase II to begin RNA synthesis at the correct site. A mammalian promoter will also contain an upstream promoter element (enhancer element), typically located within 100 to 200 base pairs upstream of the TATA box. An upstream promoter element determines the rate at which transcription is initiated and can act in either orientation. Of particular use as mammalian promoters are the promoters from mammalian viral genes, since the viral genes are often highly expressed and have a broad host range. Examples include the SV40 early promoter, mouse mammary tumor virus LTR promoter, adenovirus major late promoter, herpes simplex virus promoter, and the CMV promoter.

Typically, transcription termination and polyadenylation sequences recognized by mammalian cells are regulatory regions located 3′ to the translation stop codon and thus, together with the promoter elements, flank the coding sequence. The 3′ terminus of the mature mRNA is formed by site-specific post-translational cleavage and polyadenylation. Examples of transcription terminator and polyadenlytion signals include those derived form SV40.

The methods of introducing nucleic acid into mammalian hosts, as well as other hosts, is well known in the art, and will vary with the host cell used. Techniques include dextran-mediated transfection, calcium phosphate precipitation, polybrene mediated transfection, protoplast fusion, electroporation, viral infection, encapsulation of the polynucleotide(s) in liposomes, and direct microinjection of the DNA into nuclei.

In some methods, Wnt/β-catenin signal-promoting proteins are expressed in bacterial systems which are well known in the art.

In other methods, Wnt/β-catenin signal-promoting proteins can be produced in insect cells. Expression vectors for the transformation of insect cells, and in particular, baculovirus-based expression vectors, are well known in the art.

In some methods, Wnt/β-catenin signal-promoting proteins are produced in yeast cells. Yeast expression systems are well known in the art, and include expression vectors for Saccharomyces cerevisiae, Candida albicans and C. maltosa, Hansenula polymorpha, Kluyveromyces fragilis and K. lactis, Pichia guillerimondii and P. pastoris, Schizosaccharomyces pombe, and Yarrowia lipolytica.

A Wnt/β-catenin signal-promoting protein can also be made as a fusion protein, using techniques well known in the art. For example, for the creation of monoclonal antibodies, if the desired epitope is small, the protein can be fused to a carrier protein to form an immunogen. Alternatively, Wnt/β-catenin signal-promoting protein can be made as a fusion protein to increase expression. For example, when a protein is a shorter peptide, the nucleic acid encoding the peptide can be linked to other nucleic acid for expression purposes. Similarly, Wnt/β-catenin signal-promoting proteins of the methods and compositions herein can be linked to protein labels, such as green fluorescent protein (GFP), red fluorescent protein (RFP), yellow fluorescent protein (YFP), and blue fluorescent protein (BFP).

In one embodiment, the proteins are recombinant. A “recombinant protein” is a protein made using recombinant techniques, i.e., through the expression of a recombinant nucleic acid as depicted above. A recombinant protein is distinguished from naturally occurring protein by at least one or more characteristics. For example, the protein can be isolated or purified away from some or all of the proteins and compounds with which it is normally associated in its wild type host, and thus can be substantially pure. For example, an isolated protein is unaccompanied by at least some of the material with which it is normally associated in its natural state, typically constituting at least about 0.5%, typically at least about 5% by weight of the total protein in a given sample. A substantially pure protein comprises at least about 75% by weight of the total protein, at least about 80%, and typically at least about 90%. The definition includes the production of Wnt/β-catenin signal-promoting protein from one organism in a different organism or host cell. Alternatively, the protein can be made at a significantly higher concentration than is normally seen, through the use of a inducible promoter or high expression promoter, such that the protein is made at increased concentration levels. Alternatively, the protein can be in a form not normally found in nature, as in the addition of an epitope tag or amino acid substitutions, insertions and deletions, as discussed below.

In some methods, when the Wnt/β-catenin signal-promoting protein is to be used to generate antibodies, the protein must share at least one epitope or determinant with the full length transcription product of the nucleic acids. By “epitope” or “determinant” herein is meant a portion of a protein which will bind an antibody. Thus, in most instances, antibodies made to a smaller protein should be able to bind to the full length protein. In one embodiment, the epitope is unique; that is, antibodies generated to a unique epitope show little or no cross-reactivity.

In some methods, the antibodies provided herein can be capable of reducing or eliminating the biological function of a Wnt/β-catenin signal-promoting protein, as is described below. The addition of antibodies (either polyclonal or monoclonal) to the protein (or cells containing the protein) can reduce or eliminate the protein's activity. Generally, at least a 25% decrease in activity is observed, with typically at least about 50% and typically about a 95-100% decrease being observed.

In addition, the proteins can be variant proteins, comprising one more amino acid substitutions, insertions and deletions.

In one method, a Wnt/β-catenin signal-promoting protein is purified or isolated after expression. Proteins can be isolated or purified in a variety of ways. Standard purification methods include electrophoretic, molecular, immunological and chromatographic techniques, including ion exchange, hydrophobic, affinity, and reverse-phase HPLC chromatography, and chromatofocusing. For example, a Wnt/β-catenin signal-promoting protein can be purified using a standard anti-Wnt or anti-β-catenin protein antibody column. Ultrafiltration and diafiltration techniques, in conjunction with protein concentration, are also useful. For general guidance in suitable purification techniques, see Scopes, Protein Purification, Springer-Verlag, NY, 1982, incorporated herein by reference in its entirety. The degree of purification necessary will vary depending on the use of the protein. In some instances no purification will be necessary.

Once the gene product of the Wnt/β-catenin signal-promoting gene is made, binding assays can be done. These methods comprise combining a Wnt/β-catenin signal-promoting protein and a candidate bioactive agent, and determining the binding of the candidate agent to the Wnt/β-catenin signal-promoting protein. Methods utilize a human Wnt/β-catenin signal-promoting protein, although other mammalian proteins can also be used, including rodents (mice, rats, hamsters, guinea pigs), farm animals (cows, sheep, pigs, horses) and primates. These latter methods can be used for the development of animal models of human disease. In some methods, variant or derivative Wnt/β-catenin signal-promoting proteins can be used, including deletion Wnt/β-catenin signal-promoting proteins as outlined above.

The assays herein utilize Wnt/β-catenin signal-promoting proteins as defined herein. In some assays, portions of proteins can be utilized. In other assays, portions having different activities can be used. In addition, the assays described herein can utilize either isolated Wnt/β-catenin signal-promoting proteins or cells comprising the Wnt/β-catenin signal-promoting proteins. In some methods, the protein or the candidate agent is non-diffusably bound to an insoluble support having isolated sample receiving areas (e.g., a microtiter plate or an array). The insoluble supports can be made of any composition to which the compositions can be bound, is readily separated from soluble material, and is otherwise compatible with the overall method of screening. The surface of such supports can be solid or porous and of any convenient shape. Examples of suitable insoluble supports include microtiter plates, arrays, membranes and beads. These are typically made of glass, plastic (e.g., polystyrene), polysaccharides, nylon or nitrocellulose, and Teflon™. Microtiter plates and arrays are especially convenient because a large number of assays can be carried out simultaneously, using small amounts of reagents and samples. In some cases magnetic beads and the like are included. The particular manner of binding of the composition is not crucial so long as it is compatible with the reagents and overall methods described herein, maintains the activity of the composition and is nondiffusable. Methods of binding include the use of antibodies (which do not sterically block either the ligand binding site or activation sequence when the protein is bound to the support), direct binding to ionic supports, chemical crosslinking, or by the synthesis of the protein or agent on the surface. Following binding of the protein or agent, excess unbound material is removed by washing. The sample receiving areas can then be blocked through incubation with bovine serum albumin (BSA), casein or other innocuous protein or other moiety. Also included in the methods and compositions herein are screening assays wherein solid supports are not used.

In other methods, the Wnt/β-catenin signal-promoting protein is bound to the support, and a candidate bioactive agent is added to the assay. Alternatively, the candidate agent is bound to the support and the protein is added. Novel binding agents include specific antibodies, non-natural binding agents identified in screens of chemical libraries, and peptide analogs. Of particular interest are screening assays for agents that have a low toxicity for human cells. A wide variety of assays can be used for this purpose, including labeled in vitro protein-protein binding assays, electrophoretic mobility shift assays, immunoassays for protein binding, functional assays (such as phosphorylation assays) and the like.

The determination of the binding of the candidate bioactive agent to a Wnt/β-catenin signal-promoting protein can be done in a number of ways. In some methods, the candidate bioactive agent is labeled, and binding determined directly. For example, this can be done by attaching all or a portion of a Wnt/β-catenin signal-promoting protein to a solid support, adding a labeled candidate agent (for example a fluorescent label), washing off excess reagent, and determining whether the label is present on the solid support. Various blocking and washing steps can be utilized.

By “labeled” herein is meant that the compound is either directly or indirectly labeled with a label which provides a detectable signal, e.g., radioisotope, fluorescers, enzyme, antibodies, particles such as magnetic particles, chemiluminescers, or specific binding molecules. Specific binding molecules include pairs, such as biotin and streptavidin, digoxin and antidigoxin. For the specific binding members, the complementary member would normally be labeled with a molecule which provides for detection, in accordance with known procedures, as outlined above. The label can directly or indirectly provide a detectable signal.

In some methods, only one of the components is labeled. For example, the proteins (or proteinaceous candidate agents) can be labeled at tyrosine positions using ¹²⁵I, or with fluorophores. Alternatively, more than one component can be labeled with different labels; using ¹²⁵I for the proteins, for example, and a fluorophor for the candidate agents.

In other methods, the binding of the candidate bioactive agent is determined through the use of competitive binding assays. In this method, the competitor is a binding moiety known to bind to the target molecule such as an antibody, peptide, binding partner, or ligand. Under certain circumstances, there can be competitive binding as between the bioactive agent and the binding moiety, with the binding moiety displacing the bioactive agent. This assay can be used to determine candidate agents which interfere with binding between proteins and the competitor.

In some methods, the candidate bioactive agent is labeled. Either the candidate bioactive agent, or the competitor, or both, is added first to the protein for a time sufficient to allow binding, if present. Incubations can be performed at any temperature which facilitates optimal activity, typically between about 4° C. and 40° C. Incubation periods are selected for optimum activity, but can also be optimized to facilitate rapid high through put screening. Typically between 0.1 and 1 hour will be sufficient. Excess reagent is generally removed or washed away. The second component is then added, and the presence or absence of the labeled component is followed, to indicate binding.

In other methods, the competitor is added first, followed by the candidate bioactive agent. Displacement of the competitor is an indication that the candidate bioactive agent is binding to the Wnt/β-catenin signal-promoting protein and thus is capable of binding to, and potentially modulating, the activity of the protein. In this method, either component can be labeled. For example, if the competitor is labeled, the presence of label in the wash solution indicates displacement by the agent. Alternatively, if the candidate bioactive agent is labeled, the presence of the label on the support indicates displacement.

In other methods, the candidate bioactive agent is added first, with incubation and washing, followed by the competitor. The absence of binding by the competitor can indicate that the bioactive agent is bound to the Wnt/β-catenin signal-promoting protein with a higher affinity. Thus, if the candidate bioactive agent is labeled, the presence of the label on the support, coupled with a lack of competitor binding, can indicate that the candidate agent is capable of binding to the protein.

Competitive binding methods can also be run as differential screens. These methods can comprise a Wnt/β-catenin signal-promoting protein and a competitor in a first sample. A second sample comprises a candidate bioactive agent, a Wnt/β-catenin signal-promoting protein and a competitor. The binding of the competitor is determined for both samples, and a change, or difference in binding between the two samples indicates the presence of an agent capable of binding to the Wnt/β-catenin signal-promoting protein and potentially modulating its activity. If the binding of the competitor is different in the second sample relative to the first sample, the agent is capable of binding to the protein.

Other methods utilize differential screening to identify drug candidates that bind to the native Wnt/β-catenin signal-promoting protein, but cannot bind to modified proteins. The structure of the protein can be modeled, and used in rational drug design to synthesize agents that interact with that site. Drug candidates that affect Wnt/β-catenin signaling bioactivity are also identified by screening drugs for the ability to either enhance or reduce the activity of the protein.

In some methods, screening for agents that modulate the activity of proteins are performed. In general, this will be done on the basis of the known biological activity of the Wnt/β-catenin signal-promoting protein. In these methods, a candidate bioactive agent is added to a sample of the protein, as above, and an alteration in the biological activity of the protein is determined. “Modulating the activity” includes an increase in activity, a decrease in activity, or a change in the type or kind of activity present. Thus, in these methods, the candidate agent should both bind to a Wnt/β-catenin signal-promoting polypeptide (although this may not be necessary), and alter its biological or biochemical activity as defined herein. The methods include both in vitro screening methods, as are generally outlined above, and in vivo screening of cells for alterations in the presence, distribution, activity or amount of the protein.

Some methods comprise combining a Wnt/β-catenin signal-promoting polypeptide sample and a candidate bioactive agent, then evaluating the effect on Wnt/β-catenin signaling activity to enhance tissue regenerating activity. By “Wnt/β-catenin signaling activity” or grammatical equivalents herein is meant one of Wnt/β-catenin signaling biological activities, including, but not limited to, its ability to affect tissue regenerating activity. One activity herein is the capability to bind to a target gene, or modulate Wnt/β-catenin signaling, for example, wherein Wnt/β-catenin signaling is induced or maintained.

In other methods, the activity of the Wnt/β-catenin signal-promoting protein is increased; in other methods, the activity of the Wnt/β-catenin signal-promoting protein is decreased. Thus, bioactive agents that are antagonists are useful in some methods, and bioactive agents that are agonists are useful in other methods.

Methods for screening for bioactive agents capable of modulating the activity of a Wnt/β-catenin signal-promoting protein are provided. These methods comprise adding a candidate bioactive agent, as defined above, to a cell comprising proteins. Cell types include almost any cell. The cells contain a recombinant nucleic acid that encodes a Wnt/β-catenin signal-promoting protein. In one method, a library of candidate agents are tested on a plurality of cells. The effect of the candidate agent on Wnt/β-catenin signaling activity is then evaluated.

Positive controls and negative controls can be used in the assays. All control and test samples are performed in at least triplicate to obtain statistically significant results. Incubation of all samples is for a time sufficient for the binding of the agent to the protein. Following incubation, all samples are washed free of non-specifically bound material and the amount of bound, generally labeled agent determined. For example, where a radiolabel is employed, the samples can be counted in a scintillation counter to determine the amount of bound compound.

A variety of other reagents can be included in the screening assays. These include reagents like salts, neutral proteins (e.g., albumin and detergents) which can be used to facilitate optimal protein-protein binding and/or reduce non-specific or background interactions. Reagents that otherwise improve the efficiency of the assay, (such as protease inhibitors, nuclease inhibitors, anti-microbial agents) can also be used. The mixture of components can be added in any order that provides for the requisite binding.

The components provided herein for the assays provided herein can also be combined to form kits. The kits can be based on the use of the protein and/or the nucleic acid encoding the Wnt/β-catenin signal-promoting proteins. Assays regarding the use of nucleic acids are further described below.

(C) Animal Models

In one method, nucleic acids which encode Wnt/β-catenin signal-promoting proteins or their modified forms can also be used to generate either transgenic animals, including “knock-in” and “knock out” animals which, in turn, are useful in the development and screening of therapeutically useful reagents. A non-human transgenic animal (e.g., a mouse or rat) is an animal having cells that contain a transgene, which transgene is introduced into the animal or an ancestor of the animal at a prenatal, e.g., an embryonic stage. A transgene is a DNA which is integrated into the genome of a cell from which a transgenic animal develops, and can include both the addition of all or part of a gene or the deletion of all or part of a gene. In some methods, cDNA encoding a Wnt/β-catenin signal-promoting protein can be used to clone genomic DNA encoding a Wnt/β-catenin signal-promoting protein in accordance with established techniques and the genomic sequences used to generate transgenic animals that contain cells which either express (or overexpress) or suppress the desired DNA. Methods for generating transgenic animals, particularly animals such as mice or rats, have become conventional in the art and are described, for example, in U.S. Pat. Nos. 4,736,866 and 4,870,009, each incorporated herein by reference in their entirety. Typically, particular cells would be targeted for a Wnt/β-catenin signal-promoting protein transgene incorporation with tissue-specific enhancers. Transgenic animals that include a copy of a transgene encoding a Wnt/β-catenin signal-promoting protein introduced into the germ line of the animal at an embryonic stage can be used to examine the effect of increased expression of the desired nucleic acid. Such animals can be used as tester animals for reagents thought to confer protection from, for example, pathological conditions associated with its overexpression. In accordance with this facet, an animal is treated with the reagent and a reduced incidence of the pathological condition, compared to untreated animals bearing the transgene, would indicate a potential therapeutic intervention for the pathological condition. Similarly, non-human homologues of a Wnt/β-catenin signal-promoting protein can be used to construct a transgenic animal comprising a protein “knock out” animal which has a defective or altered gene encoding a Wnt/β-catenin signal-promoting protein as a result of homologous recombination between the endogenous gene encoding a Wnt/β-catenin signal-promoting protein and altered genomic DNA encoding the protein introduced into an embryonic cell of the animal. For example, cDNA encoding a Wnt/β-catenin signal-promoting protein can be used to clone genomic DNA encoding the protein in accordance with established techniques. A portion of the genomic DNA encoding a Wnt/β-catenin signal-promoting protein can be deleted or replaced with another gene, such as a gene encoding a selectable marker which can be used to monitor integration. Typically, several kilobases of unaltered flanking DNA (both at the 5′ and 3′ ends) are included in the vector (see, e.g., Thomas and Capecchi, Cell 51:503, 1987, incorporated herein by reference in its entirety, for a description of homologous recombination vectors). The vector is introduced into an embryonic stem cell line (e.g., by electroporation) and cells in which the introduced DNA has homologously recombined with the endogenous DNA are selected (see, e.g., Li et al., Cell 69:915, 1992, incorporated herein by reference in its entirety). The selected cells are then injected into a blastocyst of an animal (e.g., a mouse or rat) to form aggregation chimeras (see, e.g., Bradley, in Teratocarcinomas and Embryonic Stem Cells: A Practical Approach, E. J. Robertson, ed. (IRL, Oxford, 1987), pp. 113-152). A chimeric embryo can then be implanted into a suitable pseudopregnant female foster animal and the embryo brought to term to create a “knock out” animal. Progeny harboring the homologously recombined DNA in their germ cells can be identified by standard techniques and used to breed animals in which all cells of the animal contain the homologously recombined DNA. Knockout animals can be characterized for instance, for their ability to defend against certain pathological conditions and for their development of pathological conditions due to absence of a Wnt/β-catenin signal-promoting polypeptide.

Animal models exhibiting Wnt/β-catenin signaling related disorder-like symptoms can be engineered by utilizing, for example, Wnt/β-catenin signal-promoting polypeptide sequences in conjunction with techniques for producing transgenic animals that are well known to those of skill in the art. For example, gene sequences can be introduced into, and overexpressed in, the genome of the animal of interest, or, if endogenous target gene sequences are present, they can either be overexpressed or, alternatively, can be disrupted in order to underexpress or inactivate target gene expression.

In order to overexpress a target gene sequence, the coding portion of the target gene sequence can be ligated to a regulatory sequence which is capable of driving gene expression in the animal and cell type of interest. Such regulatory regions will be well known to those of skill in the art, and can be utilized in the absence of undue experimentation.

For underexpression of an endogenous target gene sequence, such a sequence can be isolated and engineered such that when reintroduced into the genome of the animal of interest, the endogenous target gene alleles will be inactivated. The engineered target gene sequence is introduced via gene targeting such that the endogenous target sequence is disrupted upon integration of the engineered target sequence into the animal's genome.

Animals of any species, including, but not limited to, mice, rats, rabbits, guinea pigs, pigs, micro-pigs, goats, and non-human primates, e.g., baboons, monkeys, and chimpanzees can be used to generate animal models of Wnt/β-catenin signaling related disorders or being a perpetually desired state of the Wnt/β-catenin signaling.

(D) Nucleic Acid Based Therapeutics

Nucleic acids encoding Wnt/β-catenin signal-promoting polypeptides, antagonists or agonists can also be used in gene therapy. Broadly speaking, a gene therapy vector is an exogenous polynucleotide which produces a medically useful phenotypic effect upon the mammalian cell(s) into which it is transferred. A vector can or can not have an origin of replication. For example, it is useful to include an origin of replication in a vector for propagation of the vector prior to administration to a patient. However, the origin of replication can often be removed before administration if the vector is designed to integrate into host chromosomal DNA or bind to host mRNA or DNA. Vectors used in gene therapy can be viral or nonviral. Viral vectors are usually introduced into a patient as components of a virus. Nonviral vectors, typically dsDNA, can be transferred as naked DNA or associated with a transfer-enhancing vehicle, such as a receptor-recognition protein, lipoamine, or cationic lipid.

Viral vectors, such as retroviruses, adenoviruses, adenoassociated viruses and herpes viruses, are often made up of two components, a modified viral genome and a coat structure surrounding it (see generally Smith et al., Ann. Rev. Microbiol. 49:807-838, 1995, incorporated herein by reference in its entirety), although sometimes viral vectors are introduced in naked form or coated with proteins other than viral proteins. Most current vectors have coat structures similar to a wildtype virus. This structure packages and protects the viral nucleic acid and provides the means to bind and enter target cells. However, the viral nucleic acid in a vector designed for gene therapy is changed in many ways. The goals of these changes are to disable growth of the virus in target cells while maintaining its ability to grow in vector form in available packaging or helper cells, to provide space within the viral genome for insertion of exogenous DNA sequences, and to incorporate new sequences that encode and enable appropriate expression of the gene of interest. Thus, vector nucleic acids generally comprise two components: essential cis-acting viral sequences for replication and packaging in a helper line and the transcription unit for the exogenous gene. Other viral functions are expressed in trans in a specific packaging or helper cell line.

Nonviral nucleic acid vectors used in gene therapy include plasmids, RNAs, antisense oligonucleotides (e.g., methylphosphonate or phosphorothiolate), polyamide nucleic acids, interfering RNA (RNAi), hairpin RNA, and yeast artificial chromosomes (YACs). Such vectors typically include an expression cassette for expressing a protein or RNA. The promoter in such an expression cassette can be constitutive, cell type-specific, stage-specific, and/or modulatable (e.g., by hormones such as glucocorticoids; MMTV promoter). Transcription can be increased by inserting an enhancer sequence into the vector. Enhancers are cis-acting sequences of between 10 to 300 bp that increase transcription by a promoter. Enhancers can effectively increase transcription when either 5′ or 3′ to the transcription unit. They are also effective if located within an intron or within the coding sequence itself. Typically, viral enhancers are used, including SV40 enhancers, cytomegalovirus enhancers, polyoma enhancers, and adenovirus enhancers. Enhancer sequences from mammalian systems are also commonly used, such as the mouse immunoglobulin heavy chain enhancer.

Gene therapy vectors can be delivered in vivo by administration to an individual patient, typically by systemic administration (e.g., intravenous, intraperitoneal, intramuscular, subdermal, or intracranial infusion) or topical application. Alternatively, vectors can be delivered to cells ex vivo, such as cells explanted from an individual patient (e.g., lymphocytes, bone marrow aspirates, tissue biopsy) or universal donor hematopoietic stem cells, followed by reimplantation of the cells into a patient, usually after selection for cells which have incorporated the vector.

### Modulating Signaling in Wnt/β-Catenin Signaling Pathway

(A) Assays for Modulators of Wnt/β-Catenin Signaling

In numerous embodiments of this invention, the level of Wnt/β-catenin signaling will be modulated in a cell by administering to the cell, in vivo or in vitro, any of a large number of Wnt/β-catenin signal-promoting molecules, e.g., polypeptides, antibodies, amino acids, nucleotides, lipids, carbohydrates, or any organic or inorganic molecule.

To identify molecules capable of modulating Wnt/β-catenin signaling, assays will be performed to detect the effect of various compounds on Wnt/β-catenin signaling activity in a cell. Wnt/β-catenin signaling can be assessed using a variety of in vitro and in vivo assays to determine functional, chemical, and physical effects, e.g., measuring the binding of Wnt or β-catenin to other molecules (e.g., radioactive binding to Wnt or β-catenin), measuring protein and/or RNA levels of Wnt/β-catenin signaling that provides a tissue regeneration activity, or measuring other aspects of pathway signaling, e.g., phosphorylation levels, transcription levels, receptor activity, ligand binding and the like. Such assays can be used to test for both activators and inhibitors of Wnt/β-catenin signaling. Modulators thus identified are useful for, e.g., many diagnostic and therapeutic applications.

The Wnt/β-catenin signaling in the assay will typically be a recombinant or naturally occurring polypeptide or a conservatively modified variant thereof. Alternatively, the Wnt/β-catenin signaling in the assay will be derived from a eukaryote and include an amino acid subsequence having amino acid sequence identity to the naturally occurring Wnt/β-catenin signaling. Generally, the amino acid sequence identity will be at least 70%, optionally at least 75%, 85%, or 86%, 87%, 88%, 89%, 90%, 91%, 92%, 93%, 94%, 95%, 96%, 97%, 98% or greater. Optionally, the polypeptide of the assays will comprise a domain of a Wnt/β-catenin signal-promoting polypeptide. In certain embodiments, a domain of Wnt or β-catenin protein is bound to a solid substrate and used, e.g., to isolate any molecules that can bind to and/or modulate their activity. In certain embodiments, a domain of a Wnt/β-catenin signal-promoting polypeptide, e.g., an N-terminal domain, a C-terminal domain, is fused to a heterologous polypeptide, thereby forming a chimeric polypeptide. Such chimeric polypeptides are also useful, e.g., in assays to identify modulators of an Wnt/β-catenin signaling.

“Identical” or percent “identity,” in the context of two or more nucleic acids or polypeptide sequences, refers to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region (e.g., nucleotide sequence encoding a collectin described herein or amino acid sequence of a collectin described herein), when compared and aligned for maximum correspondence over a comparison window or designated region) as measured using a BLAST or BLAST 2.0 sequence comparison algorithms with default parameters described below, or by manual alignment and visual inspection (see, e.g., NCBI web site). Such sequences are then said to be “substantially identical.” This term also refers to, or can be applied to, the compliment of a test sequence. The term also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window,” as used herein, includes reference to a segment of any one of the number of contiguous positions selected from the group consisting of from 20 to 600, usually about 50 to about 200, more usually about 100 to about 150 in which a sequence can be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math., 1981, 2:482, by the homology alignment algorithm of Needleman & Wunsch, J. Mol. Biol., 1970, 48:443, by the search for similarity method of Pearson & Lipman, Proc. Nat'l Acad. Sci. USA, 1988, 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology, Ausubel et al., eds. 1995 supplement)).

A preferred example of algorithm that is suitable for determining percent sequence identity and sequence similarity are the BLAST and BLAST 2.0 algorithms, which are described in Altschul et al., Nuc. Acids Res., 1977, 25:3389-3402 and Altschul et al., J. Mol. Biol., 1990, 215:403-410, respectively. BLAST and BLAST 2.0 are used, with the parameters described herein, to determine percent sequence identity for the nucleic acids and proteins of the invention. Software for performing BLAST analyses is publicly available through the National Center for Biotechnology Information (http:/www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 1989, 89:10915) alignments (B) of 50, expectation (E) of 10, M=5, N=−4, and a comparison of both strands.

“Polypeptide”, “peptide”, and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

“Amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids can be referred to herein by either their commonly known three letter symbols or by the one-letter symbols recommended by the IUPAC-IUB Biochemical Nomenclature Commission. Nucleotides, likewise, can be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

Macromolecular structures such as polypeptide structures can be described in terms of various levels of organization. For a general discussion of this organization, see, e.g., Alberts et al., Molecular Biology of the Cell (3rd ed., 1994) and Cantor and Schimmel, Biophysical Chemistry Part I: The Conformation of Biological Macromolecules (1980). “Primary structure” refers to the amino acid sequence of a particular peptide. “Secondary structure” refers to locally ordered, three dimensional structures within a polypeptide. These structures are commonly known as domains, e.g., enzymatic domains, extracellular domains, transmembrane domains, pore domains, and cytoplasmic tail domains. Domains are portions of a polypeptide that form a compact unit of the polypeptide and are typically 15 to 350 amino acids long. Exemplary domains include domains with enzymatic activity, e.g., a kinase domain. Typical domains are made up of sections of lesser organization such as stretches of β-sheet and α-helices. “Tertiary structure” refers to the complete three dimensional structure of a polypeptide monomer. “Quaternary structure” refers to the three dimensional structure formed by the noncovalent association of independent tertiary units. Anisotropic terms are also known as energy terms.

A particular nucleic acid sequence also implicitly encompasses “splice variants.” Similarly, a particular protein encoded by a nucleic acid implicitly encompasses any protein encoded by a splice variant of that nucleic acid. “Splice variants,” as the name suggests, are products of alternative splicing of a gene. After transcription, an initial nucleic acid transcript can be spliced such that different (alternate) nucleic acid splice products encode different polypeptides. Mechanisms for the production of splice variants vary, but include alternate splicing of exons. Alternate polypeptides derived from the same nucleic acid by read-through transcription are also encompassed by this definition. Any products of a splicing reaction, including recombinant forms of the splice products, are included in this definition.

The phrase “stringent hybridization conditions” refers to conditions under which a probe will hybridize to its target subsequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-110° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions can also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency. Additional guidelines for determining hybridization parameters are provided in numerous reference, e.g., Ausubel et al, supra.

For PCR, a temperature of about 36° C. is typical for low stringency amplification, although annealing temperatures can vary between about 32° C. and 48° C. depending on primer length. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include a denaturation phase of 90° C.-95° C. for 30 sec-2 min., an annealing phase lasting 30 sec.-2 min., and an extension phase of about 72° C. for 1-2 min. Protocols and guidelines for low and high stringency amplification reactions are provided, e.g., in Innis et al PCR Protocols, A Guide to Methods and Applications, Academic Press, Inc. N.Y. (1990).

Samples or assays that are treated with a potential Wnt/β-catenin signaling inhibitor or activator are compared to control samples without the test compound, to examine the extent of modulation. Control samples (untreated with activators or inhibitors) are assigned a relative activity value of 100. Inhibition of Wnt/β-catenin signaling is achieved when the Wnt/β-catenin signaling activity value relative to the control is about 90%, optionally about 50%, optionally about 25-0%. Activation of a Wnt/β-catenin signaling is achieved when the Wnt/β-catenin signaling activity value relative to the control is about 110%, optionally about 150%, 200-500%, or about 1000-2000%.

The effects of the test compounds upon the function of the polypeptides can be measured by examining any of the parameters described above. Any suitable physiological change that affects Wnt/β-catenin signaling activity can be used to assess the influence of a test compound on the polypeptides of this invention. When the functional consequences are determined using intact cells or animals, one can also measure a variety of effects such as changes in cell growth or changes in cell-cell interactions.

Modulators of Wnt/β-catenin signaling that act by modulating gene expression can also be identified. For example, a host cell containing a Wnt signaling or β-catenin protein of interest is contacted with a test compound for a sufficient time to effect any interactions, and then the level of gene expression is measured. The amount of time to effect such interactions can be empirically determined, such as by running a time course and measuring the level of transcription as a function of time. The amount of transcription can be measured using any method known to those of skill in the art to be suitable. For example, mRNA expression of the protein of interest can be detected using Northern blots or by detecting their polypeptide products using immunoassays.

(B) Assays for Wnt/β-Catenin Signaling Compounds

In certain embodiments, assays will be performed to identify molecules that physically interact with Wnt or β-catenin. Such molecules can be any type of molecule, including polypeptides, polynucleotides, amino acids, nucleotides, carbohydrates, lipids, or any other organic or inorganic molecule. Such molecules can represent molecules that normally interact with Wnt or β-catenin or can be synthetic or other molecules that are capable of interacting with Wnt or β-catenin and that can potentially be used as lead compounds to identify classes of molecules that can interact with and/or modulate Wnt/β-catenin signaling. Such assays can represent physical binding assays, such as affinity chromatography, immunoprecipitation, two-hybrid screens, or other binding assays, or can represent genetic assays.

In any of the binding or functional assays described herein, in vivo or in vitro, Wnt/β-catenin signaling, or any derivative, variation, homolog, or fragment of Wnt signaling or β-catenin, can be used. Preferably, the Wnt/β-catenin signaling protein has at least about 85% identity to the amino acid sequence of the naturally occurring Wnt/β-catenin signaling protein. In numerous embodiments, a fragment of a Wnt or β-catenin protein is used. Such fragments can be used alone, in combination with other Wnt/β-catenin signaling protein fragments, or in combination with sequences from heterologous proteins, e.g., the fragments can be fused to a heterologous polypeptides, thereby forming a chimeric polypeptide.

Compounds that interact with Wnt/β-catenin signaling can be isolated based on an ability to specifically bind to a Wnt or β-catenin or fragment thereof. In numerous embodiments, the Wnt or β-catenin or protein fragment will be attached to a solid support. In one embodiment, affinity columns are made using the Wnt or β-catenin polypeptide, and physically-interacting molecules are identified. It will be apparent to one of skill that chromatographic techniques can be performed at any scale and using equipment from many different manufactures (e.g., Pharmacia Biotechnology). In addition, molecules that interact with Wnt or β-catenin in vivo can be identified by co-immunoprecipitation or other methods, i.e., immunoprecipitating Wnt or β-catenin using anti-Wnt or anti-β-catenin antibodies from a cell or cell extract, and identifying compounds, e.g., proteins, that are precipitated along with the Wnt or β-catenin. Such methods are well known to those of skill in the art and are taught, e.g., in Ausubel et al., 1994; Sambrook et al., Molecular Cloning: A Laboratory Manual, Cold Spring Harbor Press, NY., 1989; and Harlow and Lane, Antibodies: A Laboratory Manual, Cold Spring Harbor Press, NY., 1989.

(C) Increasing Wnt or α-Catenin Protein Activity Levels in Cells

In certain embodiments, this invention provides methods of treating neoplastic disease, allogeneic tissue rejection, or graft vs. host disease by increasing Wnt or β-catenin, or Wnt/β-catenin signaling or protein levels in a cell. Typically, such methods are used to increase a reduced level of Wnt or β-catenin protein, e.g., a reduced level in a stem cell, progenitor cell, or differentiated cell population, and can be performed in any of a number of ways, e.g., increasing the copy number of Wnt or β-catenin genes or increasing the level of Wnt or β-catenin mRNA, protein, or protein activity in a cell. Preferably, the level of protein activity is increased to a level typical of a normal, cell, but the level can be increased to any level that is sufficient to increase Wnt/β-catenin signaling in an stem cell, progenitor cell, or differentiated cell population, including to levels above or below those typical of normal cells. Preferably, such methods involve the use of activators of Wnt/β-catenin signaling, where an “activator of Wnt/β-catenin signaling” is a molecule that acts to increase Wnt or β-catenin gene polynucleotide levels, polypeptide levels and/or protein activity. Such activators can include, but are not limited to, small molecule activators of Wnt/β-catenin signaling.

In preferred embodiments, Wnt or β-catenin protein levels or Wnt/β-catenin signaling will be increased so as to increase stem cell, progenitor cell, or differentiated cell population in vivo in a vertebrate subject or to treat a degenerative muscle disease or neurodegenerative disease in a vertebrate subject as a result of decreased Wnt/β-catenin signaling levels. The proliferation of a cell refers to the rate at which the cell or population of cells divides, or to the extent to which the cell or population of cells divides or increases in number. Proliferation can reflect any of a number of factors, including the rate of cell growth and division and the rate of cell death. Without being bound by the following offered theory, it is suggested that the amplification and/or overexpression of the Wnt/β-catenin signaling in stem cell, progenitor cell, or differentiated cell population to treat a degenerative muscle disease or neurodegenerative disease in a vertebrate subject. Activation of tissue regenerating activity via Wnt or β-catenin protein is useful to treat a degenerative muscle disease or neurodegenerative disease in a vertebrate subject. The ability of any of the present compounds to affect Wnt/β-catenin signaling activity can be determined based on any of a number of factors, including, but not limited to, a level of Wnt or β-catenin polynucleotide, e.g., mRNA or gDNA, the level of Wnt or β-catenin polypeptide, the degree of binding of a compound to a Wnt or β-catenin polynucleotide or polypeptide, Wnt or β-catenin protein intracellular localization, or any functional properties of Wnt or β-catenin protein, such as the ability of Wnt or β-catenin protein activity to enhance tissue regenerating activity, or to treat a degenerative muscle disease or neurodegenerative disease in a vertebrate subject.

(D) Regulators of Wnt or β-Catenin Polynucleotides

In certain embodiments, Wnt/β-catenin signaling activity is regulated by the use of antisense polynucleotide, i.e., a nucleic acid complementary to, and which can preferably hybridize specifically to, a coding mRNA nucleic acid sequence, e.g., Wnt/β-catenin signaling mRNA, or a subsequence thereof. Binding of the antisense polynucleotide to the mRNA reduces the translation and/or stability of the Wnt/β-catenin signaling mRNA.

In the context of this invention, antisense polynucleotides can comprise naturally-occurring nucleotides, or synthetic species formed from naturally-occurring subunits or their close homologs. Antisense polynucleotides can also have altered sugar moieties or inter-sugar linkages. Exemplary among these are the phosphorothioate and other sulfur containing species which are known for use in the art. All such analogs are comprehended by this invention so long as they function effectively to hybridize with Wnt/β-catenin signaling mRNA.

Such antisense polynucleotides can readily be synthesized using recombinant means, or can be synthesized in vitro. Equipment for such synthesis is sold by several vendors, including Applied Biosystems. The preparation of other oligonucleotides such as phosphorothioates and alkylated derivatives is also well known to those of skill in the art.

In addition to antisense polynucleotides, ribozymes can be used to target and inhibit transcription of Wnt/β-catenin signaling protein. A ribozyme is an RNA molecule that catalytically cleaves other RNA molecules. Different kinds of ribozymes have been described, including group I ribozymes, hammerhead ribozymes, hairpin ribozymes, RNAse P, and axhead ribozymes (see, e.g., Castanotto et al., Adv. in Pharmacology 25: 289-317, 1994 for a general review of the properties of different ribozymes).

The general features of hairpin ribozymes are described, e.g., in Hampel et al., Nucl. Acids Res., 18: 299-304, 1990; Hampel et al., European Patent Publication No. 0 360 257, 1990; U.S. Pat. No. 5,254,678. Methods of preparing are well known to those of skill in the art (see, e.g., Wong-Staal et al., WO 94/26877; Ojwang et al., Proc. Natl. Acad. Sci. USA, 90: 6340-6344, 1993; Yamada et al., Human Gene Therapy 1: 39-45, 1994; Leavitt et al., Proc. Natl. Acad. Sci. USA, 92: 699-703, 1995; Leavitt et al., Human Gene Therapy 5: 1151-120, 1994; and Yamada et al., Virology 205: 121-126, 1994).

Wnt/β-catenin signaling protein activity can also be increased by the addition of an activator of the Wnt or β-catenin protein, or an inhibitor of β-catenin independent signaling, e.g., inhibitors of wnt5a or wnt5b. This can be accomplished in any of a number of ways, including by providing a dominant negative β-catenin independent signaling polypeptide, e.g., a form of β-catenin independent signaling protein that itself has no activity and which, when present in the same cell as a functional Wnt/β-catenin signaling protein, reduces or eliminates the β-catenin independent signaling protein activity, e.g., dominant negative forms of wnt5a or wnt5b. Design of dominant negative forms is well known to those of skill and is described, e.g., in Herskowitz, Nature 329:219-22, 1987. Also, inactive polypeptide variants (muteins) can be used, e.g., by screening for the ability to inhibit β-catenin independent signaling protein activity. Methods of making muteins are well known to those of skill (see, e.g., U.S. Pat. Nos. 5,486,463; 5,422,260; 5,116,943; 4,752,585; and 4,518,504). In addition, any small molecule, e.g., any peptide, amino acid, nucleotide, lipid, carbohydrate, or any other organic or inorganic molecule can be screened for the ability to bind to or inhibit β-catenin independent signaling protein activity, as described below.

(E) Modulators and Binding Compounds

The compounds tested as modulators of a Wnt/β-catenin signaling protein can be any small chemical compound, or a biological entity, such as a protein, sugar, nucleic acid or lipid. Typically, test compounds will be small chemical molecules and peptides. Essentially any chemical compound can be used as a potential modulator or binding compound in the assays of the invention, although most often compounds can be dissolved in aqueous or organic (especially DMSO-based) solutions. The assays are designed to screen large chemical libraries by automating the assay steps and providing compounds from any convenient source to assays, which are typically run in parallel (e.g., in microtiter formats on microtiter plates in robotic assays). It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs, Switzerland) and the like.

In one preferred embodiment, high throughput screening methods involve providing a combinatorial chemical or peptide library containing a large number of potential therapeutic compounds (potential modulator or binding compounds). Such “combinatorial chemical libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175; Furka, Int. J. Pept. Prot. Res. 37:487-493, 1991; and Houghton et al., Nature 354: 84-88, 1991). Other chemistries for generating chemical diversity libraries can also be used. Such chemistries include, but are not limited to: peptoids (e.g., PCT Publication No. WO 91/19735), encoded peptides (e.g., PCT Publication No. WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90:6909-6913, 1993), vinylogous polypeptides (Hagihara et al., J. Amer. Chem. Soc. 114:6568, 1992), nonpeptidal peptidomimetics with glucose scaffolding (Hirschmann et al., J. Amer. Chem. Soc. 114:9217-9218, 1992), analogous organic syntheses of small compound libraries (Chen et al., J. Amer. Chem. Soc. 116:2661, 1994), oligocarbamates (Cho et al., Science 261:1303, 1993), and/or peptidyl phosphonates (Campbell et al., J. Org. Chem. 59:658, 1994), nucleic acid libraries (see Ausubel, Berger and Sambrook, all supra), peptide nucleic acid libraries (see, e.g., U.S. Pat. No. 5,539,083), antibody libraries (see, e.g., Vaughn et al., Nature Biotechnology 14:309-314, 1996; and PCT/US96/10287), carbohydrate libraries (see, e.g., Liang et al., Science, 274:1520-1522, 1996; and U.S. Pat. No. 5,593,853), small organic molecule libraries (see, e.g., benzodiazepines, Baum, C&EN, page 33, Jan. 18, 1993; isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J.; Tripos, Inc., St. Louis, Mo.; 3D Pharmaceuticals, Exton, Pa.; Martek Biosciences, Columbia, Md., etc.).

(F) Solid State and Soluble High Throughput Assays

In one embodiment, the invention provides soluble assays using molecules such as an N-terminal or C-terminal domain either alone or covalently linked to a heterologous protein to create a chimeric molecule. In another embodiment, the invention provides solid phase based in vitro assays in a high throughput format, where a domain, chimeric molecule, Wnt/β-catenin signaling protein, or cell or tissue expressing a Wnt/β-catenin signaling protein is attached to a solid phase substrate.

In the high throughput assays of the invention, it is possible to screen up to several thousand different modulators in a single day. In particular, each well of a microtiter plate can be used to run a separate assay against a selected potential modulator, or, if concentration or incubation time effects are to be observed, every 5-10 wells can test a single modulator. Thus, a single standard microtiter plate can assay about 100 (e.g., 96) modulators. If 1536 well plates are used, then a single plate can easily assay from about 100 to about 1500 different compounds. It is possible to assay several different plates per day; assay screens for up to about 6,000-20,000 different compounds is possible using the integrated systems of the invention. More recently, microfluidic approaches to reagent manipulation have been developed.

The molecule of interest can be bound to the solid state component, directly or indirectly, via covalent or non covalent linkage, e.g., via a tag. The tag can be any of a variety of components. In general, a molecule which binds the tag (a tag binder) is fixed to a solid support, and the tagged molecule of interest is attached to the solid support by interaction of the tag and the tag binder.

A number of tags and tag binders can be used, based upon known molecular interactions well described in the literature. For example, where a tag has a natural binder, for example, biotin, protein A, or protein G, it can be used in conjunction with appropriate tag binders (avidin, streptavidin, neutravidin, the Fc region of an immunoglobulin, etc.) Antibodies to molecules with natural binders such as biotin are also widely available and appropriate tag binders; see, SIGMA Immunochemicals 1998 catalogue SIGMA, St. Louis Mo.).

Similarly, any haptenic or antigenic compound can be used in combination with an appropriate antibody to form a tag/tag binder pair. Thousands of specific antibodies are commercially available and many additional antibodies are described in the literature. For example, in one common configuration, the tag is a first antibody and the tag binder is a second antibody which recognizes the first antibody.

Synthetic polymers, such as polyurethanes, polyesters, polycarbonates, polyureas, polyamides, polyethyleneimines, polyarylene sulfides, polysiloxanes, polyimides, and polyacetates can also form an appropriate tag or tag binder. Many other tag/tag binder pairs are also useful in assay systems described herein, as would be apparent to one of skill upon review of this disclosure.

Common linkers such as peptides, polyethers, and the like can also serve as tags, and include polypeptide sequences, such as poly-gly sequences of between about 5 and 200 amino acids. Such flexible linkers are known to persons of skill in the art. For example, poly(ethelyne glycol) linkers are available from Shearwater Polymers, Inc. Huntsville, Ala. These linkers optionally have amide linkages, sulfhydryl linkages, or heterofunctional linkages.

Tag binders are fixed to solid substrates using any of a variety of methods currently available. Solid substrates are commonly derivatized or functionalized by exposing all or a portion of the substrate to a chemical reagent which fixes a chemical group to the surface which is reactive with a portion of the tag binder. For example, groups which are suitable for attachment to a longer chain portion would include amines, hydroxyl, thiol, and carboxyl groups. Aminoalkylsilanes and hydroxyalkylsilanes can be used to functionalize a variety of surfaces, such as glass surfaces. The construction of such solid phase biopolymer arrays is well described in the literature. See, e.g., Merrifield, J. Am. Chem. Soc. 85:2149-2154, 1993 (describing solid phase synthesis of, e.g., peptides); Geysen et al., J. Immun. Meth. 102:259-274, 1987 (describing synthesis of solid phase components on pins); Frank & Doring, Tetrahedron 44:6031-6040, 1988 (describing synthesis of various peptide sequences on cellulose disks); Fodor et al., Science 251: 767-777, 1991; Sheldon et al., Clinical Chemistry 39:718-719, 1993; and Kozal et al., Nature Medicine 2:753-759, 1996 (all describing arrays of biopolymers fixed to solid substrates). Nonchemical approaches for fixing tag binders to substrates include other common methods, such as heat, cross-linking by UV radiation, and the like.

(G) Rational Drug Design Assays

Yet another assay for compounds that modulate Wnt/β-catenin signaling protein activity involves computer assisted drug design, in which a computer system is used to generate a three-dimensional structure of a Wnt/β-catenin signaling protein based on the structural information encoded by its amino acid sequence. The input amino acid sequence interacts directly and actively with a pre-established algorithm in a computer program to yield secondary, tertiary, and quaternary structural models of the protein. The models of the protein structure are then examined to identify regions of the structure that have the ability to bind. These regions are then used to identify compounds that bind to the protein.

The three-dimensional structural model of the protein is generated by entering protein amino acid sequences of at least 10 amino acid residues or corresponding nucleic acid sequences encoding a Wnt/β-catenin signaling polypeptide into the computer system. The nucleotide sequence encoding the polypeptide, or the amino acid sequence thereof, and conservatively modified versions thereof, of the naturally occurring gene sequence. The amino acid sequence represents the primary sequence or subsequence of the protein, which encodes the structural information of the protein. At least 10 residues of the amino acid sequence (or a nucleotide sequence encoding 10 amino acids) are entered into the computer system from computer keyboards, computer readable substrates that include, but are not limited to, electronic storage media (e.g., magnetic diskettes, tapes, cartridges, and chips), optical media (e.g., CD ROM), information distributed by internet sites, and by RAM. The three-dimensional structural model of the protein is then generated by the interaction of the amino acid sequence and the computer system, using software known to those of skill in the art.

The amino acid sequence represents a primary structure that encodes the information necessary to form the secondary, tertiary and quaternary structure of the protein of interest. The software looks at certain parameters encoded by the primary sequence to generate the structural model. These parameters are referred to as “energy terms,” and primarily include electrostatic potentials, hydrophobic potentials, solvent accessible surfaces, and hydrogen bonding. Secondary energy terms include van der Waals potentials. Biological molecules form the structures that minimize the energy terms in a cumulative fashion. The computer program is therefore using these terms encoded by the primary structure or amino acid sequence to create the secondary structural model.

The tertiary structure of the protein encoded by the secondary structure is then formed on the basis of the energy terms of the secondary structure. The user at this point can enter additional variables such as whether the protein is membrane bound or soluble, its location in the body, and its cellular location, e.g., cytoplasmic, surface, or nuclear. These variables along with the energy terms of the secondary structure are used to form the model of the tertiary structure. In modeling the tertiary structure, the computer program matches hydrophobic faces of secondary structure with like, and hydrophilic faces of secondary structure with like.

Once the structure has been generated, potential modulator binding regions are identified by the computer system. Three-dimensional structures for potential modulators are generated by entering amino acid or nucleotide sequences or chemical formulas of compounds, as described above. The three-dimensional structure of the potential modulator is then compared to that of the Wnt/β-catenin signaling protein to identify compounds that bind to the protein. Binding affinity between the protein and compound is determined using energy terms to determine which compounds have an enhanced probability of binding to the protein.

Computer systems are also used to screen for mutations, polymorphic variants, alleles and interspecies homologs of Wnt/β-catenin signaling genes. Such mutations can be associated with disease states or genetic traits. GeneChip™ and related technology can also be used to screen for mutations, polymorphic variants, alleles and interspecies homologs. Once the variants are identified, diagnostic assays can be used to identify patients having such mutated genes. Identification of the mutated Wnt/β-catenin signaling genes involves receiving input of a first nucleic acid or amino acid sequence of the naturally occurring Wnt/β-catenin signaling induced gene, respectively, and conservatively modified versions thereof. The sequence is entered into the computer system as described above. The first nucleic acid or amino acid sequence is then compared to a second nucleic acid or amino acid sequence that has substantial identity to the first sequence. The second sequence is entered into the computer system in the manner described above. Once the first and second sequences are compared, nucleotide or amino acid differences between the sequences are identified. Such sequences can represent allelic differences in various Wnt/β-catenin signaling genes, and mutations associated with disease states and genetic traits.

### Diagnostic Methods

In addition to assays, the creation of animal models, and nucleic acid based therepeutics, identification of important genes allows the use of these genes in diagnosis (e.g., diagnosis of cell states and abnormal cell conditions). Disorders based on mutant or variant Wnt/β-catenin signaling genes can be determined. Methods for identifying cells containing variant Wnt/β-catenin signaling genes comprising determining all or part of the sequence of at least one endogeneous genes in a cell are provided. As will be appreciated by those in the art, this can be done using any number of sequencing techniques. Methods of identifying the genotype of an individual comprising determining all or part of the sequence of at least one Wnt/β-catenin signaling gene of the individual are also provided. This is generally done in at least one tissue of the individual, and can include the evaluation of a number of tissues or different samples of the same tissue. The method can include comparing the sequence of the sequenced mutant Wnt gene or β-catenin gene to a known Wnt gene or β-catenin gene, i.e., a wild-type gene.

The sequence of all or part of the Wnt/β-catenin signaling gene can then be compared to the sequence of a known Wnt/β-catenin signaling gene to determine if any differences exist. This can be done using any number of known sequence identity programs, such as Bestfit, and others outlined herein. In some methods, the presence of a difference in the sequence between the Wnt/β-catenin signaling gene of the patient and the known Wnt/β-catenin signaling gene is indicative of a disease state or a propensity for a disease state, as outlined herein.

Similarly, diagnosis of stem cell, progenitor cell, or differentiated cell population states can be done using the methods and compositions herein. By evaluating the gene expression profile of stem cell, progenitor cell, or differentiated cell population from a patient, the stem cell, progenitor cell, or differentiated cell population state can be determined. This is particularly useful to verify the action of a drug, for example an immunosuppressive drug. Other methods comprise administering the drug to a patient and removing a cell sample, particularly of stem cell, progenitor cell, or differentiated cell population, from the patient. The gene expression profile of the cell is then evaluated, as outlined herein, for example by comparing it to the expression profile from an equivalent sample from a healthy individual. In this manner, both the efficacy (i.e., whether the correct expression profile is being generated from the drug) and the dose (is the dosage correct to result in the correct expression profile) can be verified.

The present discovery relating to the role of Wnt/β-catenin signaling in enhancing a tissue regenerating activity, e.g., treating degenerative muscle disease or neurodegenerative disease in a vertebrate subject. In one method, the Wnt/β-catenin signaling proteins, and particularly Wnt/β-catenin signaling protein fragments, are useful in the study or treatment of conditions which are mediated by various disease states, i.e., to diagnose, treat or prevent degenerative muscle disease or neurodegenerative disease, which can be treated with compounds activating tissue regeneration.

Methods of modulating tissue regeneration states in cells or organisms are provided. Some methods comprise administering to a cell an anti-Wnt signaling promoting protein antibody or anti-O-catenin signal-promoting protein antibody or other agent identified herein or by the methods provided herein, that reduces or eliminates the biological activity of the endogeneous Wnt/β-catenin signaling protein. Alternatively, the methods comprise administering to a cell or organism a recombinant nucleic acid encoding a Wnt/β-catenin signal-promoting protein or modulator including anti-sense nucleic acids. As will be appreciated by those in the art, this can be accomplished in any number of ways. In some methods, the activity Wnt/β-catenin signaling is increased by increasing the amount or activity of Wnt/β-catenin signal-promoting protein in the cell, for example by overexpressing the endogeneous Wnt/β-catenin signal-promoting protein or by administering a Wnt/β-catenin signal-promoting gene, using known gene therapy techniques, for example. In one method, the gene therapy techniques include the incorporation of the exogenous gene using enhanced homologous recombination (EHR), for example as described in PCT/US93/03868, hereby incorporated by reference in its entirety.

Methods for diagnosing a stem cell, progenitor cell, or differentiated cell population activity related condition in an individual are provided. The methods comprise measuring the activity of Wnt/β-catenin signal-promoting protein in a tissue from the individual or patient, which can include a measurement of the amount or specific activity of the protein. This activity is compared to the activity of Wnt/β-catenin signal-promoting protein from either an unaffected second individual or from an unaffected tissue from the first individual. When these activities are different, the first individual can be at risk for a stem cell, progenitor cell, or differentiated cell population activity mediated disorder.

Furthermore, nucleotide sequences encoding a Wnt/β-catenin signal-promoting protein can also be used to construct hybridization probes for mapping the gene which encodes that Wnt/β-catenin signal-promoting protein and for the genetic analysis of individuals with genetic disorders. The nucleotide sequences provided herein can be mapped to a chromosome and specific regions of a chromosome using known techniques, such as in situ hybridization, linkage analysis against known chromosomal markers, and hybridization screening with libraries.

### Antibodies

“Antibody” is used in the broadest sense and specifically covers polyclonal antibodies, monoclonal antibodies, antibody compositions with polyepitopic specificity, bispecific antibodies, diabodies, and single-chain molecules, as well as antibody fragments (e.g., Fab, F(ab′)₂, and Fv), so long as they exhibit the desired biological activity. Antibodies can be labeled/conjugated to toxic or non-toxic moieties. Toxic moieties include, for example, bacterial toxins, viral toxins, radioisotopes, and the like. Antibodies can be labeled for use in biological assays (e.g., radioisotope labels, fluorescent labels) to aid in detection of the antibody. Antibodies can also be labeled/conjugated for diagnostic or therapeutic purposes, e.g., with radioactive isotopes that deliver radiation directly to a desired site for applications such as radioimmunotherapy (Garmestani. et al., Nucl Med Biol, 28:409, 2001), imaging techniques and radioimmunoguided surgery or labels that allow for in vivo imaging or detection of specific antibody/antigen complexes. Antibodies can also be conjugated with toxins to provide an immunotoxin (see, Kreitman, RJ Adv Drug Del Rev, 31:53, 1998).

“Monoclonal antibody” refers to an antibody obtained from a population of substantially homogeneous antibodies, i.e., the individual antibodies comprising the population are identical except for possible naturally occurring mutations that can be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention can be made by the hybridoma method first described by Kohler et al., Nature, 256: 495, 1975, or can be made by recombinant DNA methods (see, e.g., U.S. Pat. No. 4,816,567, Cabilly et al.). The “monoclonal antibodies” can also be isolated from phage antibody libraries using the techniques described in Clackson et al., 624-628, 1991, and Marks et al., J. Mol. Biol., 222:581-597, 1991, for example.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity. (Cabilly et al., supra; Morrison et al., Proc. Natl. Acad. Sci. USA, 81:6851-6855, 1984).

“Humanized” forms of non-human (e.g., murine) antibodies are chimeric immunoglobulins, immunoglobulin chains or fragments thereof (such as Fv, Fab, Fab′, F(ab′)2 or other antigen-binding subsequences of antibodies) which contain minimal sequence derived from non-human immunoglobulin. For the most part, humanized antibodies are human immunoglobulins (recipient antibody) in which residues from a complementary-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, humanized antibodies can comprise residues which are found neither in the recipient antibody nor in the imported CDR or framework sequences. These modifications are made to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin sequence. The humanized antibody optimally also will comprise at least a portion of an immunoglobulin constant region (Fc), typically that of a human immunoglobulin. For further details, see Jones et al., Nature 321:522-525, 1986; Reichmann et al., Nature 332:323-329, 1988; and Presta, Curr. Op. Struct. Biol. 2:593-596, 1992. The humanized antibody includes a Primatized™ antibody wherein the antigen-binding region of the antibody is derived from an antibody produced by immunizing macaque monkeys with the antigen of interest.

Amino acids from the variable regions of the mature heavy and light chains of immunoglobulins are designated Hx and Lx respectively, where x is a number designating the position of an amino acids according to the scheme of Kabat et al., 1987 and 1991, Sequences of Proteins of Immunological Interest (National Institutes of Health, Bethesda, Md.). Kabat et al list many amino acid sequences for antibodies for each subclass, and list the most commonly occurring amino acid for each residue position in that subclass. Kabat et al use a method for assigning a residue number to each amino acid in a listed sequence, and this method for assigning residue numbers has become standard in the field. Kabat et al.'s scheme is extendible to other antibodies not included in the compendium by aligning the antibody in question with one of the consensus sequences in Kabat et al The use of the Kabat et al. numbering system readily identifies amino acids at equivalent positions in different antibodies. For example, an amino acid at the L50 position of a human antibody occupies the equivalence position to an amino acid position L50 of a mouse antibody

“Non-immunogenic in a human” means that upon contacting the polypeptide of interest in a physiologically acceptable carrier and in a therapeutically effective amount with the appropriate tissue of a human, no state of sensitivity or resistance to the polypeptide of interest is demonstrable upon the second administration of the polypeptide of interest after an appropriate latent period (e.g., 8 to 14 days).

“Neutralizing antibody” refers to an antibody which is able to block or significantly reduce an effector function of wild type or mutant Wnt/β-catenin signal-promoting. For example, a neutralizing antibody can inhibit or reduce Wnt or β-catenin activation by an agonist antibody, as determined, for example, in a Wnt/β-catenin signaling assay, or other assays taught herein or known in the art.

In some methods, the Wnt/β-catenin signal-promoting proteins can be used to generate polyclonal and monoclonal antibodies to Wnt/β-catenin signal-promoting proteins, which are useful as described herein. A number of immunogens are used to produce antibodies that specifically bind Wnt/β-catenin signal-promoting polypeptides. Full-length Wnt/β-catenin signal-promoting polypeptides are suitable immunogens. Typically, the immunogen of interest is a peptide of at least about 3 amino acids, more typically the peptide is at least 5 amino acids in length, the fragment is at least 10 amino acids in length and typically the fragment is at least 15 amino acids in length. The peptides can be coupled to a carrier protein (e.g., as a fusion protein), or are recombinantly expressed in an immunization vector. Antigenic determinants on peptides to which antibodies bind are typically 3 to 10 amino acids in length. Naturally occurring polypeptides are also used either in pure or impure form. Recombinant polypeptides are expressed in eukaryotic or prokaryotic cells and purified using standard techniques. The polypeptide, or a synthetic version thereof, is then injected into an animal capable of producing antibodies. Either monoclonal or polyclonal antibodies can be generated for subsequent use in immunoassays to measure the presence and quantity of the polypeptide.

These antibodies find use in a number of applications. For example, the Wnt/β-catenin signal-promoting antibodies can be coupled to standard affinity chromatography columns and used to purify Wnt/β-catenin signal-promoting proteins as further described below. The antibodies can also be used as blocking polypeptides, as outlined above, since they will specifically bind to the Wnt/β-catenin signal-promoting protein.

The anti-Wnt protein or anti-β-catenin protein antibodies can comprise polyclonal antibodies. Methods for producing polyclonal antibodies are known to those of skill in the art. In brief, an immunogen, for example, a purified polypeptide, a polypeptide coupled to an appropriate carrier (e.g., GST and keyhole limpet hemocyanin), or a polypeptide incorporated into an immunization vector such as a recombinant vaccinia virus (see, U.S. Pat. No. 4,722,848) is mixed with an adjuvant and animals are immunized with the mixture. The animal's immune response to the immunogen preparation is monitored by taking test bleeds and determining the titer of reactivity to the polypeptide of interest. When appropriately high titers of antibody to the immunogen are obtained, blood is collected from the animal and antisera are prepared. Further fractionation of the antisera to enrich for antibodies reactive to the polypeptide is performed where desired. See, e.g., Coligan, Current Protocols in Immunology, Wiley/Greene, NY, 1991; and Harlow and Lane, supra, each incorporated herein by reference in their entirety.

Antibodies, including binding fragments and single chain recombinant versions thereof, against predetermined fragments of Wnt/β-catenin signal-promoting proteins are raised by immunizing animals, e.g., with conjugates of the fragments with carrier proteins as described above.

The phrase “immune cell response” refers to the response of immune system cells to external or internal stimuli (e.g., antigen, cytokines, chemokines, and other cells) producing biochemical changes in the immune cells that result in immune cell migration, killing of target cells, phagocytosis, production of antibodies, other soluble effectors of the immune response, and the like.

“Immune response” refers to the concerted action of lymphocytes, antigen presenting cells, phagocytic cells, granulocytes, and soluble macromolecules produced by the above cells or the liver (including antibodies, cytokines, and complement) that results in selective damage to, destruction of, or elimination from the human body of invading pathogens, cells or tissues infected with pathogens, cancerous cells, allogeneic tissue rejection, or, in cases of autoimmunity or pathological inflammation, normal human cells or tissues.

Other suitable techniques involve selection of libraries of recombinant antibodies in phage or similar vectors, for example, single chain Fv (scFv) libraries. See, Huse et al., Science 246:1275-1281, 1989; and Ward, et al., Nature 341:544-546, 1989, each incorporated herein by reference in their entirety.

The protocol described by Huse is rendered more efficient in combination with phage-display technology. See, e.g., Dower et al., WO 91/17271; McCafferty et al., WO 92/01047; and U.S. Pat. Nos. 5,871,907; 5,858,657; 5,837,242; 5,733,743; and 5,565,332, each incorporated herein by reference in their entirety. In these methods, libraries of phage are produced in which members (display packages) display different antibodies on their outer surfaces. Antibodies are usually displayed as Fv or Fab fragments. Phage displaying antibodies with a desired specificity can be selected by affinity enrichment to the antigen or fragment thereof. Phage display combined with immunized transgenic non-human animals expressing human immunoglobulin genes can be used to obtain antigen specific antibodies even when the immune response to the antigen is weak.

Also, recombinant immunoglobulins can be produced. See, U.S. Pat. No. 4,816,567; and Queen et al., Proc. Natl. Acad. Sci. USA 86:10029-10033, 1989, each incorporated herein by reference in their entirety.

Briefly, nucleic acids encoding light and heavy chain variable regions, optionally linked to constant regions, are inserted into expression vectors. The light and heavy chains can be cloned in the same or different expression vectors. The DNA segments encoding antibody chains are operably linked to control sequences in the expression vector(s) that ensure the expression of antibody chains. Such control sequences include a signal sequence, a promoter, an enhancer, and a transcription termination sequence. Expression vectors are typically replicable in the host organisms either as episomes or as an integral part of the host chromosome.

E. coli is one procaryotic host useful for expressing antibodies. Other microbial hosts suitable for use include bacilli, such as Bacillus subtilus, and other enterobacteriaceae, such as Salmonella, Serratia, and various Pseudomonas species. In these prokaryotic hosts, one can also make expression vectors, which typically contain expression control sequences compatible with the host cell (e.g., an origin of replication) and regulatory sequences such as a lactose promoter system, a tryptophan (trp) promoter system, a beta-lactamase promoter system, or a promoter system from phage lambda.

Other microbes, such as yeast, can also be used for expression. Saccharomyces is one host, with suitable vectors having expression control sequences, such as promoters, including 3-phosphoglycerate kinase or other glycolytic enzymes, and an origin of replication, termination sequences and the like as desired.

Vertebrate tissue cell culture can also be used to express and produce the antibodies (See Winnacker, From Genes to Clones, VCH Publishers, N.Y., 1987, incorporated herein by reference in its entirety). Eukaryotic cells are useful because a number of suitable host cell lines capable of secreting intact antibodies have been developed. Suitable host cells for expressing nucleic acids encoding the immunoglobulins include: monkey kidney CV1 line transformed by SV40 (COS-7, ATCC CRL 1651); human embryonic kidney line (293) (Graham et al., J. Gen. Virol. 36:59, 1977); baby hamster kidney cells (BHK, ATCC CCL 10); Chinese hamster ovary-cells-DHFR (CHO, Urlaub and Chasin, Proc. Natl. Acad. Sci. U.S.A. 77:4216, 1980); mouse sertoli cells (TM4, Mather, Biol. Reprod. 23:243-251, 1980); monkey kidney cells (CV1 ATCC CCL 70); african green monkey kidney cells (VERO-76, ATCC CRL 1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); and, TRI cells (Mather et al., Annals N.Y. Acad. Sci. 383:44-46, 1982); baculovirus cells. Each citation is incorporated herein by reference in their entirety

The vectors containing the polynucleotide sequences of interest (e.g., the heavy and light chain encoding sequences and expression control sequences) can be transferred into the host cell. Calcium chloride transfection is commonly utilized for prokaryotic cells, whereas calcium phosphate treatment or electroporation can be used for other cellular hosts. See generally Sambrook et al., Molecular Cloning: A Laboratory Manual, Cold Spring Harbor Press, 2d ed., 1989, incorporated herein by reference in its entirety.

Once expressed, the whole antibodies, their dimers, individual light and heavy chains, or other immunoglobulin forms can be purified according to standard procedures of the art, including ammonium sulfate precipitation, affinity columns, column chromatography, gel electrophoresis and the like. See generally Scopes, Protein Purification, Springer-Verlag, N.Y., 1982, incorporated herein by reference in its entirety. Substantially pure immunoglobulins are of at least about 90 to 95% homogeneity, and are typically 98 to 99% homogeneity or more.

Frequently, the polypeptides and antibodies will be labeled by joining, either covalently or non-covalently, a substance which provides for a detectable signal. A wide variety of labels and conjugation techniques are known and are reported extensively in both the scientific and patent literature. Thus, an antibody used for detecting an analyte can be directly labeled with a detectable moiety, or can be indirectly labeled by, for example, binding to the antibody a secondary antibody that is, itself directly or indirectly labeled.

Antibodies are also used for affinity chromatography in isolating Wnt/β-catenin signal-promoting proteins. Columns are prepared, e.g., with the antibodies linked to a solid support, e.g., particles, such as agarose, Sephadex, or the like, where a cell lysate is passed through the column, washed, and treated with increasing concentrations of a mild denaturant, whereby purified Wnt/β-catenin signal-promoting polypeptides are released.

### Effective Dosages

Effective doses of a composition of Wnt/β-catenin signal-promoting agent for the treatment of disease, e.g., degenerative muscle disease or neurodegenerative disease, described herein vary depending upon many different factors, including means of administration, target site, physiological state of the patient, whether the patient is human or an animal, other medications administered, and whether treatment is prophylactic or therapeutic. Usually, the patient is a human but nonhuman mammals including transgenic mammals can also be treated. Treatment dosages need to be titrated to optimize safety and efficacy.

For administration with a Wnt/β-catenin signal-promoting agent, the dosage ranges from about 0.0001 to 100 mg/kg, and more usually 0.01 to 5 mg/kg, of the host body weight. For example dosages can be 1 mg/kg body weight, 10 mg/kg body weight or 30 mg/kg body weight, or within the range of 1-30 mg/kg body weight. An exemplary treatment dosage with a Wnt/β-catenin signal-promoting agent or a β-catenin-independent signaling antagonist is 30 mg/kg body weight. An exemplary treatment regime entails administration once per every two weeks or once a month or once every 3 to 6 months. In some methods, two or more Wnt/β-catenin pathway agonist(s) and/or one or more inhibitor(s) of β-catenin-independent signaling are administered simultaneously, in which case the dosage of each Wnt/β-catenin signal-promoting agent administered falls within the ranges indicated. Multiple administrations of Wnt/β-catenin signal-promoting agent can occur. Intervals between single dosages can be weekly, monthly or yearly. Intervals can also be irregular as indicated by measuring blood levels of the Wnt/β-catenin signal-promoting agent in the patient. In some methods, dosage is adjusted to achieve a plasma enriched Wnt/β-catenin signal-promoting agent of 1-1000 μg/ml and in some methods 25-300 μg/ml. Alternatively, a Wnt/β-catenin signal-promoting agent can be administered as a sustained release formulation, in which case less frequent administration is required. Dosage and frequency vary depending on the half-life of the Wnt/β-catenin signal-promoting agent in the patient. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patent can be administered a prophylactic regime.

### Routes of Administration

Compositions of a Wnt/β-catenin signal-promoting agent for the treatment of disease, e.g., degenerative muscle disease or neurodegenerative disease, can be administered by intravesicular, intrathecal, parenteral, topical, intravenous, oral, inhalants, subcutaneous, intraarterial, intracranial, intraperitoneal, intranasal or intramuscular means. As a prophylactic/adjuvant or for treatment of disease, Wnt/β-catenin pathway agonist(s) and/or one or more inhibitor(s) of β-catenin-independent signaling target a degenerative muscle disease or neurodegenerative disease, and/or a therapeutic treatment that provides tissue regeneration. The most typical route of administration of an immunogenic agent is subcutaneous or intravenous although other routes can be equally effective. The next most common route is intramuscular injection. This type of injection is most typically performed in the arm or leg muscles. In some methods, agents are injected directly into a particular tissue where deposits have accumulated, for example injection into the bone marrow. Intramuscular injection on intravenous infusion are preferred for administration of a Wnt/β-catenin signal-promoting agent.

Agents of the invention can optionally be administered in combination with other agents that are at least partly effective in treating various diseases including degenerative muscle disease or neurodegenerative disease.

### Formulation

Compositions of a Wnt/β-catenin signal-promoting agent for the treatment of disease, e.g., degenerative muscle disease or neurodegenerative disease.

Compositions of a Wnt/β-catenin signal-promoting agent for the treatment of disease, e.g., degenerative muscle disease or neurodegenerative disease, are often administered as pharmaceutical compositions comprising an active therapeutic agent, i.e., and a variety of other pharmaceutically acceptable components. See, e.g., Alfonso R Gennaro (ed), Remington: The Science and Practice of Pharmacy, (Formerly Remington's Pharmaceutical Sciences) 20th ed., Lippincott, Williams & Wilkins, 2003, incorporated herein by reference in its entirety. The preferred form depends on the intended mode of administration and therapeutic application. The compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation can also include other carriers, adjuvants, or nontoxic, nontherapeutic, nonimmunogenic stabilizers and the like.

Pharmaceutical compositions can also include large, slowly metabolized macromolecules such as proteins, polysaccharides such as chitosan, polylactic acids, polyglycolic acids and copolymers (such as latex functionalized Sepharose™, agarose, cellulose, and the like), polymeric amino acids, amino acid copolymers, and lipid aggregates (such as oil droplets or liposomes). Additionally, these carriers can function as immunostimulating agents (i.e., adjuvants).

For parenteral administration, compositions of the invention can be administered as injectable dosages of a solution or suspension of the substance in a physiologically acceptable diluent with a pharmaceutical carrier that can be a sterile liquid such as water oils, saline, glycerol, or ethanol. Additionally, auxiliary substances, such as wetting or emulsifying agents, surfactants, pH buffering substances and the like can be present in compositions. Other components of pharmaceutical compositions are those of petroleum, animal, vegetable, or synthetic origin, for example, peanut oil, soybean oil, and mineral oil. In general, glycols such as propylene glycol or polyethylene glycol are preferred liquid carriers, particularly for injectable solutions. Wnt/β-catenin signal-promoting agent can be administered in the form of a depot injection or implant preparation which can be formulated in such a manner as to permit a sustained release of the active ingredient. An exemplary composition comprises an Wnt/β-catenin signal-promoting agent at 5 mg/mL, formulated in aqueous buffer consisting of 50 mM L-histidine, 150 mM NaCl, adjusted to pH 6.0 with HCl.

Typically, compositions are prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above. Langer, Science, 249: 1527, 1990; Hanes, Advanced Drug Delivery Reviews, 28: 97-119, 1997, incorporated herein by reference in their entirety. The agents of this invention can be administered in the form of a depot injection or implant preparation which can be formulated in such a manner as to permit a sustained or pulsatile release of the active ingredient.

Additional formulations suitable for other modes of administration include oral, intranasal, and pulmonary formulations, suppositories, and transdermal applications.

For suppositories, binders and carriers include, for example, polyalkylene glycols or triglycerides; such suppositories can be formed from mixtures containing the active ingredient in the range of 0.5% to 10%, preferably 1%-2%. Oral formulations include excipients, such as pharmaceutical grades of mannitol, lactose, starch, magnesium stearate, sodium saccharine, cellulose, and magnesium carbonate. These compositions take the form of solutions, suspensions, tablets, pills, capsules, sustained release formulations or powders and contain 10%-95% of active ingredient, preferably 25%-70%.

Topical application can result in transdermal or intradermal delivery. Topical administration can be facilitated by co-administration of the agent with cholera toxin or detoxified derivatives or subunits thereof or other similar bacterial toxins. Glenn, et al., Nature, 391: 851, 1998. Co-administration can be achieved by using the components as a mixture or as linked molecules obtained by chemical crosslinking or expression as a fusion protein.

Alternatively, transdermal delivery can be achieved using a skin patch or using transferosomes. Paul, et al., Eur. J. Immunol., 25: 3521-24, 1995; Cevc, et al., Biochem. Biophys. Acta., 1368: 201-15, 1998, incorporated herein by reference in their entirety.

The pharmaceutical compositions generally comprise a composition of the enriched Wnt/β-catenin signal-promoting agent in a form suitable for administration to a patient. The pharmaceutical compositions are generally formulated as sterile, substantially isotonic and in full compliance with all Good Manufacturing Practice (GMP) regulations of the U.S. Food and Drug Administration.

### Toxicity

Preferably, a therapeutically effective dose of a composition of the Wnt/β-catenin signal-promoting agent described herein will provide therapeutic benefit without causing substantial toxicity.

Toxicity of the Wnt/β-catenin signal-promoting agent described herein can be determined by standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. The data obtained from these cell culture assays and animal studies can be used in formulating a dosage range that is not toxic for use in human. The dosage of the Wnt/β-catenin signal-promoting agent described herein lies preferably within a range of circulating concentrations that include the effective dose with little or no toxicity. The dosage can vary within this range depending upon the dosage form employed and the route of administration utilized. The exact formulation, route of administration and dosage can be chosen by the individual physician in view of the patient's condition. (See, e.g., Fingl, et al., The Pharmacological Basis Of Therapeutics, Ch. 1, 1975), incorporated herein by reference in its entirety.

### Kits

Also within the scope of the invention are kits comprising the compositions (e.g., a Wnt/β-catenin signal-promoting agent) of the invention and instructions for use. The kit can further contain a least one additional reagent, or one or more additional human antibodies of the invention (e.g., a human antibody having a complementary activity which binds to an epitope in the antigen distinct from the first human antibody). Kits typically include a label indicating the intended use of the contents of the kit. The term label includes any writing, or recorded material supplied on or with the kit, or which otherwise accompanies the kit.

Other embodiments and uses will be apparent to one skilled in the art in light of the present disclosures.

### EXEMPLARY EMBODIMENTS

### Example 1

### Wnt/β-Catenin Signaling is Activated During Regeneration

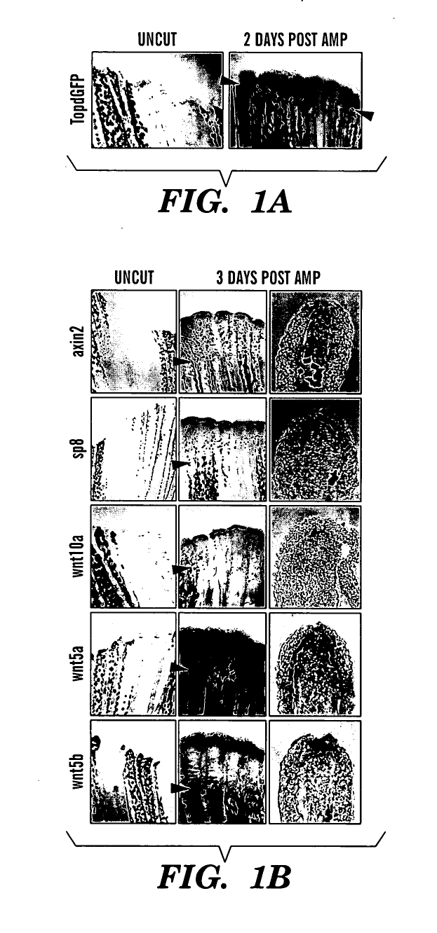

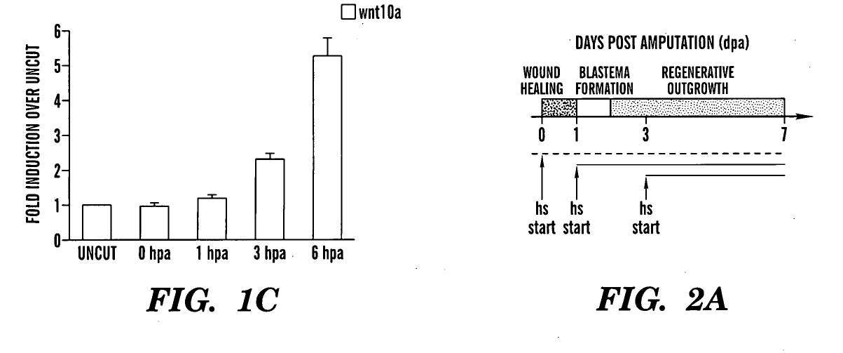

Since the zebrafish tail fin is a good model for epimorphic regeneration (Poss et al., Dev Dyn 226:202-10, 2003) and is highly amenable to experimental manipulation, we studied the role of Wnt signaling in regeneration using the zebrafish fin model. Expression of Wnt ligands and components of the β-catenin signaling pathway has been reported in regenerating amphibian and fish appendages (Caubit et al., Dev Dyn 210:1-10, 1997a; Caubit et al., Dev Dyn 208:139-48, 1997; Poss et al., Dev Dyn 219:282-6, 2000), suggesting that Wnt/β-catenin signaling is upregulated during regeneration. The endpoint of Wnt/β-catenin signaling is transcriptional regulation of target genes; however, since Wnt signaling is tightly regulated by extracellular, cytoplasmic and nuclear inhibitors, expression of Wnt ligands does not necessarily result in activation of transcription. Thus, to test whether the Wnt/β-catenin pathway is functional during zebrafish fin regeneration, we asked whether a transcriptional reporter of Wnt/β-catenin signaling, TOPdGFP (Dorsky et al., Dev Biol 241:229-37, 2002), is activated in response to fin amputation in TOPdGFP transgenic zebrafish. The present study found that TOPdGFP is detectable in the blastema of the regenerating fin at 2 days post amputation (FIG. 1A). The present study also found that the expression of axin2, which has been shown to be a direct Wnt target gene in several systems (Jho et al., Mol Cell Biol 22:1172-83, 2002; Leung et al., J Biol Chem 277:21657-65, 2002; Lustig et al., Mol Cell Biol 22:1184-93, 2002; Weidinger et al., Curr Biol 15:489-500, 2005), and sp8, which is regulated by Wnt/β-catenin signaling in fin and limb development (Kawakami et al., Development 131:4763-74, 2004), is upregulated in regenerating zebrafish tail fins (FIG. 1B).

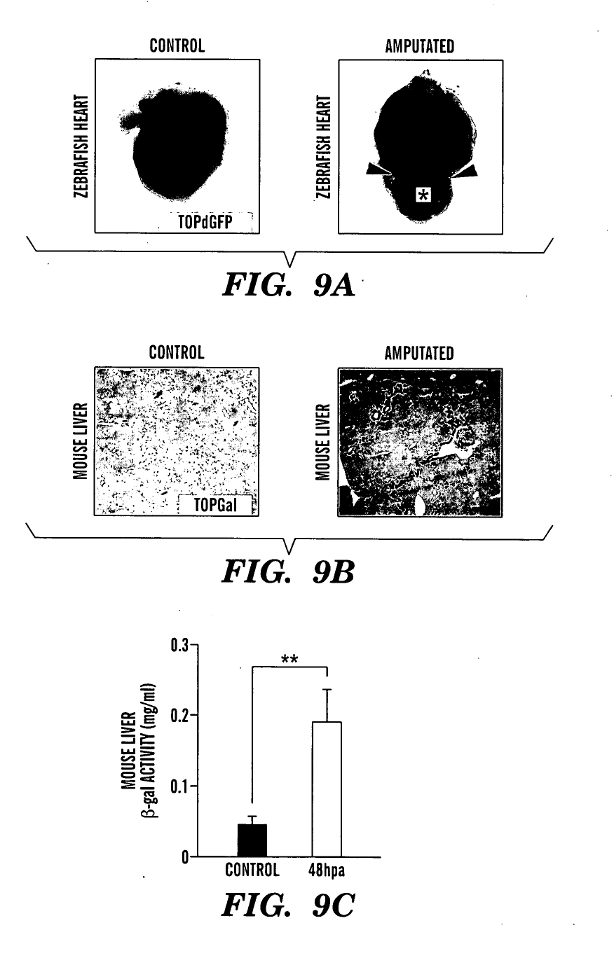

The question was: which Wnt ligands might be responsible for activation of Wnt/β-catenin signaling during regeneration of the tail fin. The present study found that wnt10a, which has been shown to activate Wnt/β-catenin signaling during limb development (Narita et al., Dev Dyn 233:282-7, 2005), is expressed early during regeneration. Expression of wnt10a is detectable in the distal tip of the blastema (FIG. 1B). Using quantitative PCR, we found that expression of wnt10a is upregulated very early during regeneration, being expressed 2.3 fold higher than in uncut fins at 3 hours post amputation (hpa) and 5.3 fold at 6 hpa (FIG. 1C). Thus, wnt10a is an excellent candidate for a Wnt ligand responsible for early activation of the β-catenin signaling pathway during fin regeneration. Interestingly, the present study found that Wnt signaling activity as detected by transgenic reporters is also upregulated during zebrafish heart and mouse liver regeneration (FIG. 9), suggesting that activation of Wnt/β-catenin signaling may be a conserved feature of regeneration.

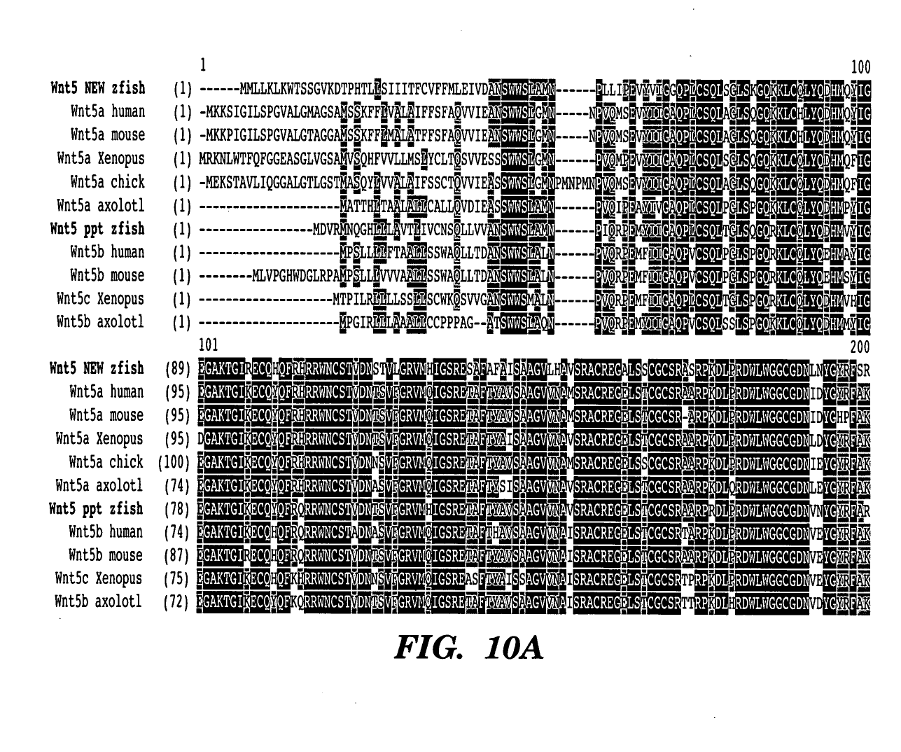

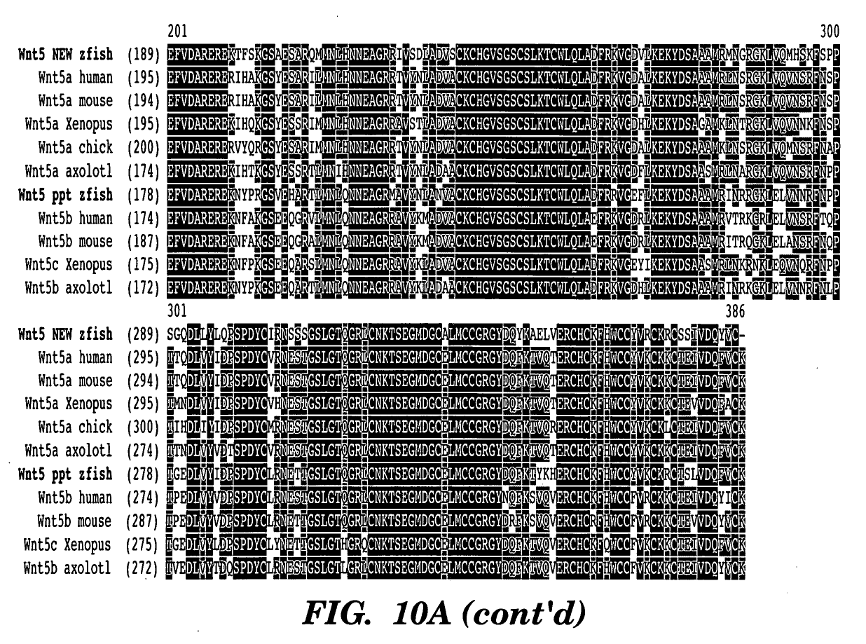

The present study also tested whether Wnts that have been shown to signal via β-catenin-independent pathways in other systems (Slusarski et al., Dev Biol 182:114-20, 1997; Veeman et al., Dev Cells 5:367-77, 2003), are expressed during zebrafish fin regeneration. The zebrafish orthologue of wnt5a (FIG. 10) was cloned and found that its expression is induced after the blastema is formed and is maintained throughout regeneration. Wnt5a is expressed in the basal epithelial layer of the regeneration epidermis as well as in the distal tip of the blastema (FIG. 1B). Wnt5b/pipetail (see FIG. 10 for nomenclature) which, like wnt5a, has been shown to signal via β-catenin-independent pathways in other systems (Westfall et al., J Cell Biol 162:889-98, 2003), is also expressed in the basal epithelial layer of the epidermis, albeit only at the very tip of the regenerate, as well as in the distal tip of the blastemal mesenchyme (FIG. 1B). These data suggest that β-catenin-independent Wnt signaling pathways, activated by Wnt5 paralogs, play a role in fin regeneration.

FIGS. 1A, 1B, and 1C show Wnt/β-catenin signaling is upregulated in regenerating zebrafish tail fins. (A) Wnt/β-catenin reporter (TOPdGFP) activity (detected by in situ hybridization for GFP RNA, blue staining) is upregulated in the blastema of regenerating fins of zebrafish homozygous for the transgene at 48 hpa (n=5, arrowheads indicate the amputation plane). Control is a non-amputated TOPdGFP fin. Likewise, at 3 dpa (n=3) and 5 dpa (n=3), TOPdGFP was still upregulated. (B) In situ hybridization of control non-amputated fins (left panel), regenerating fins at 3 dpa (middle panel), and cross-sections of fins at the same stage (right panel). The Wnt/β-catenin target genes axin2 and sp8 are expressed in the distal tip of the blastemal mesenchyme and in the basal epithelial layer of the regeneration epidermis, respectively. wnt10a is expressed in the distal tip of the blastema. Both wnt5a (see FIG. 10 for nomenclature) and wnt5b are expressed in the basal epithelial layer of the regeneration epidermis and in the distal tip of the blastema, with wnt5a extending far proximally in the basal epithelium. (C) wnt10a expression levels in uncut control and regenerating fins at 0 hpa (sample isolated immediately after fin amputation), 1 hpa, 3 hpa and 6 hpa as determined by quantitative PCR. RNA was isolated from the tips of fins of 10 wild-type fish for each time point. Expression levels were normalized to β-actin levels (normalization to 18S rRNA levels produced very similar results) and fold-induction calculated by setting the level of uncut fins to 1. qPCR was performed 4 times on the same samples, error bars represent the s.e.m.

### Example 2

### Wnt/β-Catenin Signaling is Required for Fin Regeneration

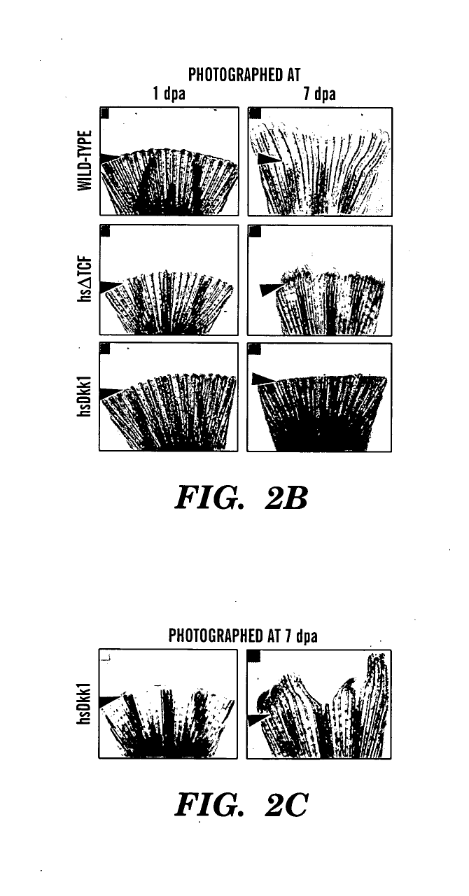

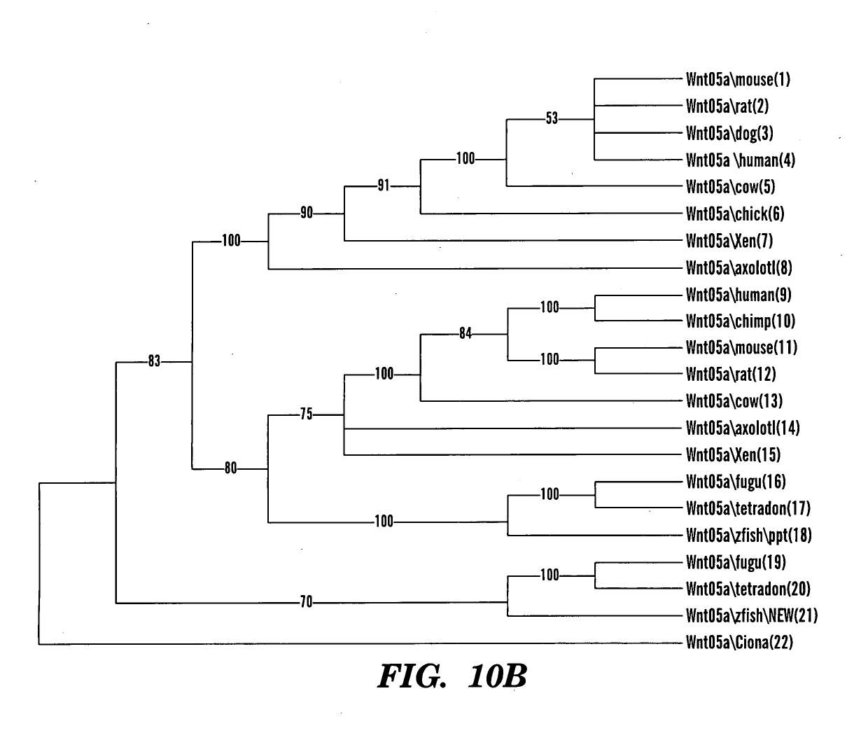

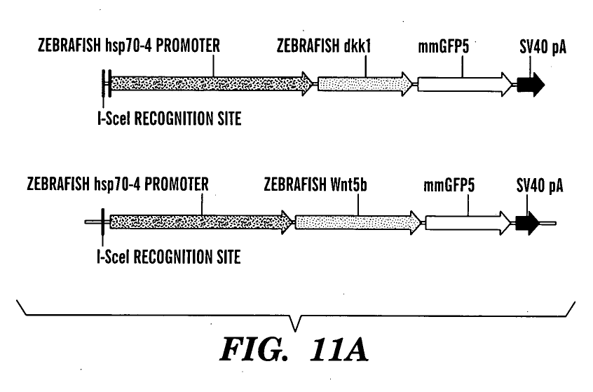

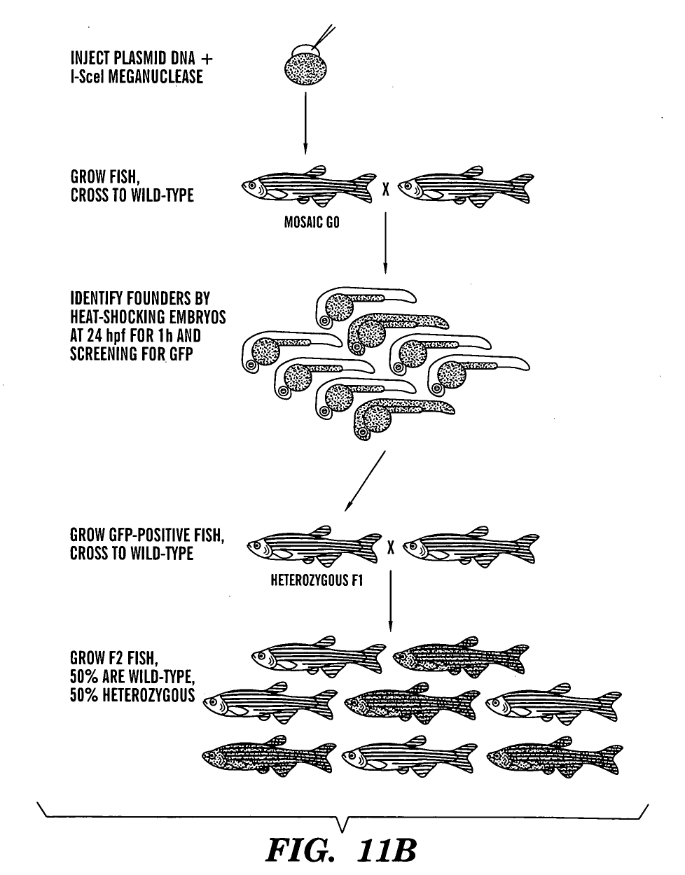

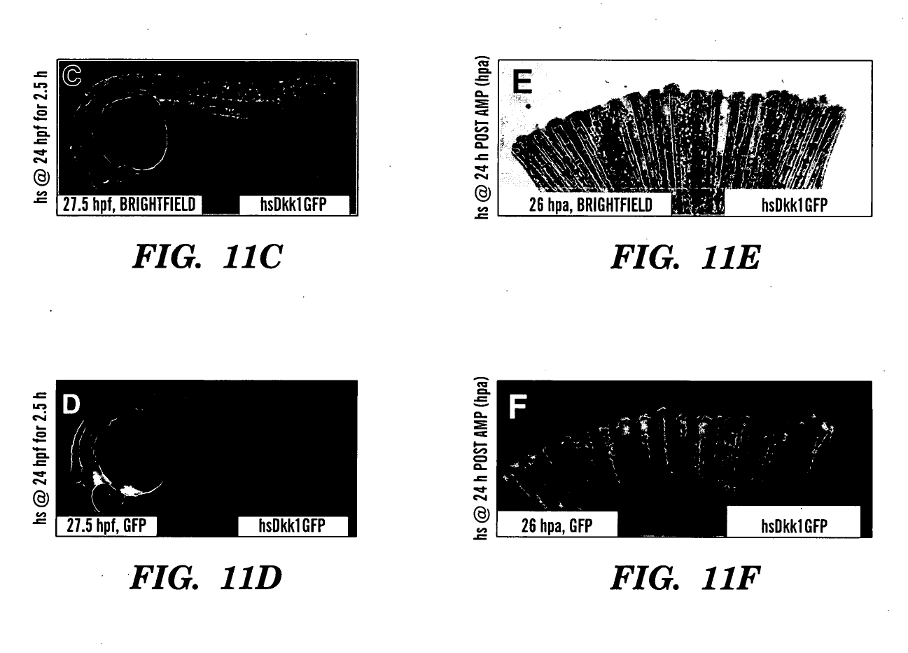

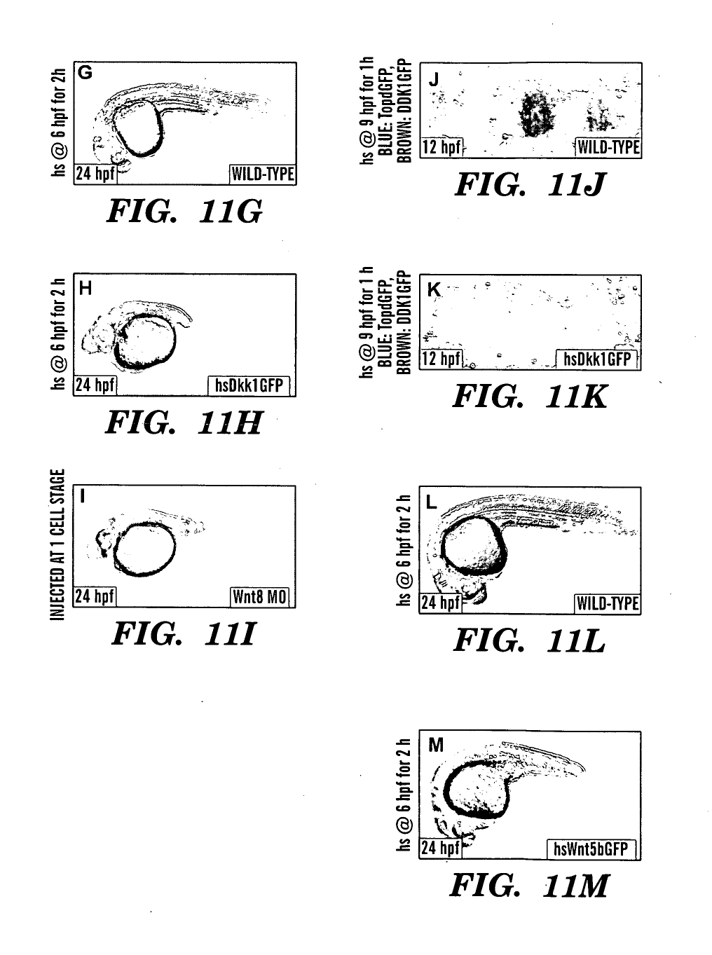

To test the requirement of Wnt/β-catenin signaling for fin regeneration a line of zebrafish was created which are transgenic for heat-shock inducible Dickkopf-1 (hsDkk1GFP; FIG. 11), a secreted inhibitor of Wnt/β-catenin signaling (Glinka et al., Nature 391:357-62, 1998). Activation of the transgene during embryogenesis phenocopies the effects of wnt8 loss-of-function (FIG. 11G-I) and is sufficient to suppress expression of the TOPdGFP Wnt/β-catenin reporter in doubly transgenic embryos 3 h after induction (FIG. 11J-K). Heat shock induces ubiquitous expression of the transgene (as monitored by GFP expression) in embryos and regenerating adult tail fins (FIG. 11C-F). Thus, this transgenic line represents an excellent tool to study the functions of Wnt/β-catenin signaling during late embryogenesis and in adults. Additionally, we employed a zebrafish line transgenic for a heat-shock inducible dominant-negative Tcf (hsΔTcfGFP), which has been shown to efficiently inhibit expression of Wnt/β-catenin target genes (Lewis et al., Development 131:1299-308, 2004). When the fish were heat-shocked two hours before fin amputation and continued to heat-shock twice daily for seven days, it was found that regeneration was completely blocked in both hsDkk1GFP and hsΔTcfGFP transgenic fish, while regeneration in heat-shocked wild-type fish was unperturbed (FIG. 2A, B).

Fin regeneration can be divided into three phases: wound healing, which happens within 24 hours post amputation (hpa) at 29° C., blastema formation (approximately 24-48 hpa), and regenerative outgrowth (starting around 48 hpa). This inducible transgenic system allowed us to test when Wnt/β-catenin signaling is required during regeneration. Interestingly, when we started to inhibit Wnt/β-catenin signaling by heat-shock after wound healing has taken place, but before the regeneration blastema has formed (24 hpa, FIG. 2A), regeneration was again completely blocked (FIG. 2C, left panel). Thus, impaired regeneration in Dkk1-overexpressing fish is not a consequence of failed wound healing, but rather is due to a specific requirement for Wnt/β-catenin signaling during blastema formation. It was also asked whether Wnt/β-catenin signaling is important for the outgrowth phase of fin regeneration. To do this, we began heat-shocking hsDkk1GFP transgenic fish at 72 hpa (FIG. 2A). These fish displayed incomplete regeneration (FIG. 2C, right panel), indicating that Wnt/β-catenin signaling is not only required for formation of the blastema, but subsequently for blastema maintenance and/or proliferation.

FIGS. 2A, 2B, and 2C show Wnt/β-catenin signaling is required for zebrafish tail fin regeneration. (A) Experimental scheme. Tail fins were amputated from wild-type, hsDkk1GFP or hsΔTCFGFP transgenic zebrafish and heat-shocks were applied twice daily for the time periods indicated by colored lines. (B) Continuous suppression of Wnt target gene expression in hsΔTCFGFP (n=15) or reduction of Wnt/β-catenin signaling in hsDkk1GFP transgenic fish (n=19) for 7 days starting shortly before amputation inhibits fin regeneration. Live fins were photographed at 1 dpa (left panel) and 7 dpa (right panel). (C) Overexpression of Dkk1 starting at 1 dpa inhibits fin regeneration (left panel, n=18); overexpression from 3 dpa results in partial inhibition of regeneration (right panel, 11/15 fins). Live fins were photographed at 7 dpa, corresponding wild-type controls regenerated normally (not shown).

### Example 3

### Wnt/β-Catenin Signaling Regulates Blastema Formation and Subsequent Proliferation

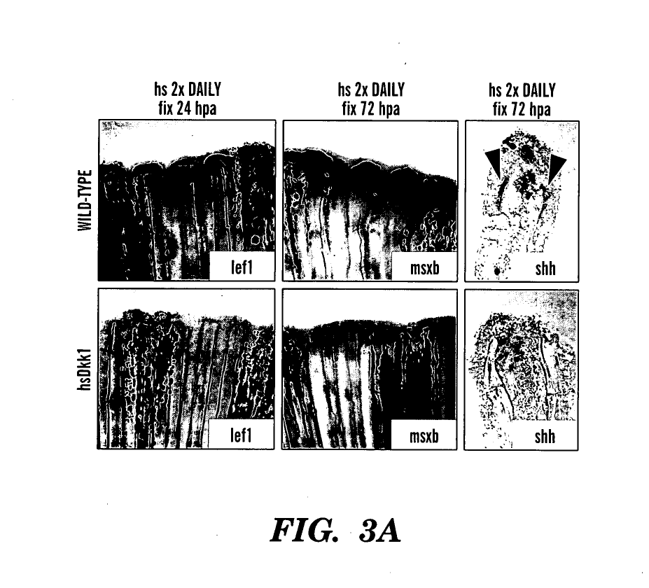

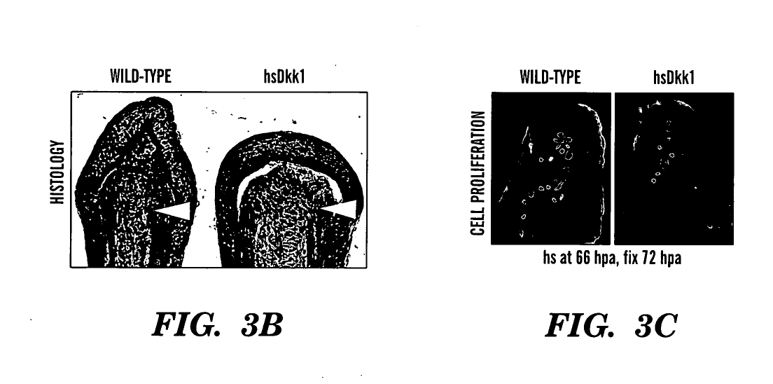

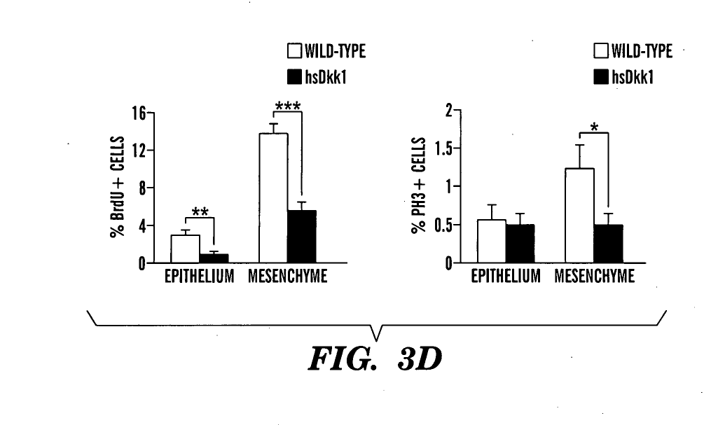

To characterize the cell biological functions of Wnt/β-catenin during fin regeneration, assays were carried out to test for specific effects of Dkk1 overexpression on cell specification and proliferation. Heatshock of hsDkk1GFP fish starting shortly before amputation results in a loss of expression of lef1, a marker for the basal epidermis (Poss et al., Dev Dyn 219:282-6, 2000), by 24 hpa, indicating that the basal layer of the wound epidermis is not specified correctly (FIG. 3A). The present study also found that expression of msxb, a marker for the mesenchymal progenitor cells of the regeneration blastema (Poss et al., Dev Biol 222:347-58, 2000), and shh, which is normally expressed within basal epidermal cells (Poss et al., Dev Biol 222:347-58, 2000), are lost by 72 hpa in Dkk1-expressing fins (FIG. 3A). Histological examination confirms that formation of the regeneration blastema is severely impaired in hsDkk1GFP fish although the wound heals properly (FIG. 3B). These data show that neither the blastema mesenchyme nor the overlying epithelium are specified correctly following loss of Wnt/β-catenin signaling.

To test whether Wnt/β-catenin signaling is in addition required for proliferation of the blastema, Wnt signaling was inhibited by a single pulse of Dkk1 expression in regenerating fins during the outgrowth phase of regeneration at 3 dpa. The present study assayed for cell proliferation 6 hours post heatshock using BrdU incorporation and staining for phosphorylated histones and found that loss of Wnt/β-catenin signaling leads to a reduction in proliferation of both the mesenchyme and the epithelium of the blastema (FIG. 3C, D). Thus, Wnt/β-catenin signaling is required for formation and subsequent proliferation of the blastema.

FIGS. 3A, 3B, 3C, and 3D show Wnt/β-catenin signaling regulates specification and proliferation of the regeneration blastema. (A) Expression of lef1, a marker for the basal epidermal layer of the regeneration epithelium, msxb, marking the mesenchymal progenitor cells of the blastema, and shh, expressed in basal epidermal cells (shown in thick sections), is strongly reduced in Dkk1-overexpressing fins. lef1 is shown at 24 hpa (n=4), msxb (n=4) and shh (n=4) at 72 hpa. Fish were heat-shocked twice daily starting shortly before amputation. (B) Hematoxylin-stained sections of tail fin regenerates at 48 hpa. Dkk1-overexpressing fins (right panel, n=6) display reduced numbers of deep mesenchymal cells of the blastema. Fish were heat-shocked twice daily starting shortly before amputation. Arrowheads indicate the plane of amputation. (C) 72 hpa regenerates stained for BrdU (red), phosphorylated histones (green) and DAPI (blue). Cell proliferation in both the mesenchyme and epithelium is decreased in Dkk1-overexpressing fins. Fish were heat-shocked once at 66 hpa and fixed at 72 hpa. (D) Quantification of the cell proliferation defects in Dkk1-overexpressing regenerating fins. The fraction of BrdU positive (left graph) and phosphorylated histone (right graph) positive cells relative to the total number of cells (DAPI positive) is shown in percent (n=11). Error bars represent the s.e.m.

### Example 4

### Wnt/β-Catenin Signaling is Sufficient to Enhance Regeneration

The next question posed was whether enhanced Wnt/β-catenin signaling is sufficient to augment regeneration. To activate Wnt/β-catenin signaling transgenic fish were used that overexpress Wnt8 after heat-shock (hsWnt8GFP) (Weidinger et al., 2005). During embryogenesis, heat-shock of these fish causes characteristic Wnt/β-catenin gain-of-function phenotypes (Weidinger et al., 2005).

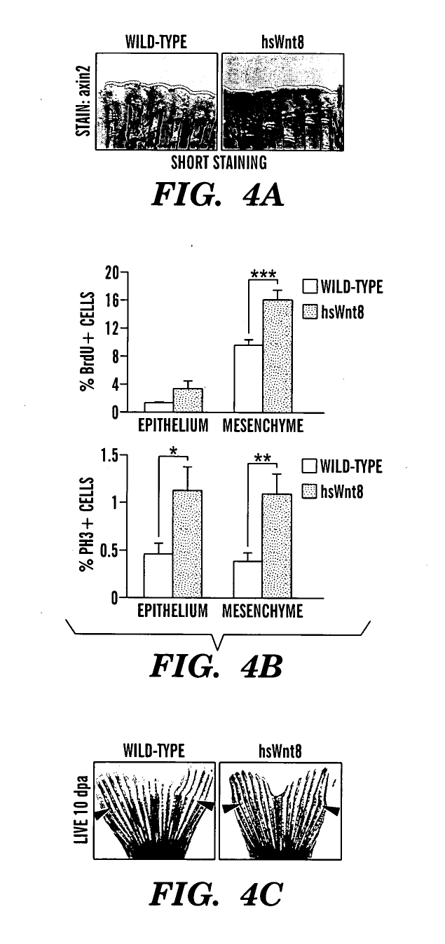

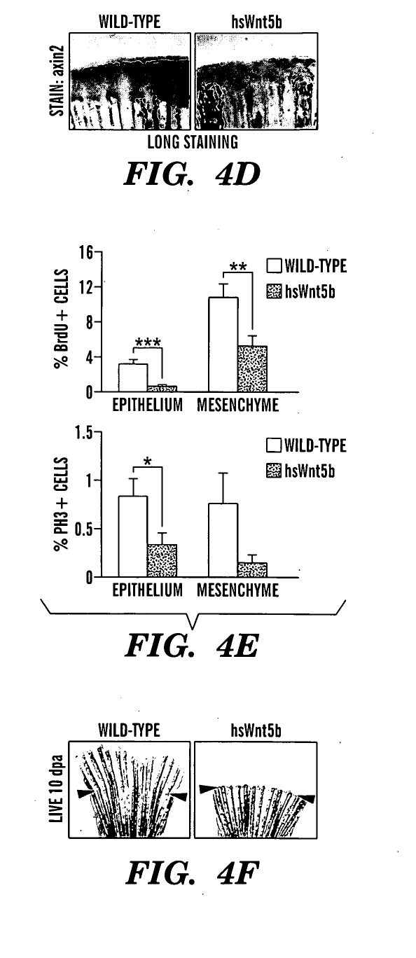

Induction of Wnt8 during fin regeneration increased expression of the Wnt/β-catenin target gene axin2 (FIG. 4A), showing that overexpression of Wnt8 in the fin is sufficient to augment β-catenin signaling. Importantly, overexpression of Wnt8 at 72 hpa significantly increased proliferation of the blastema mesenchyme and overlying epithelium 6 hours after induction of the transgene as detected by BrdU incorporation and anti-phosphorylated histone 3 antibody staining (FIG. 4B). Despite its ability to increase proliferation, overexpression of Wnt8 had no consistent effect on fin length by 10 days post amputation (FIG. 4C). However, the short half-life of Wnt proteins and the pulsed activation of the transgene raise the question of whether a more prolonged and consistent activation of the pathway might be sufficient to augment overall fin regeneration.

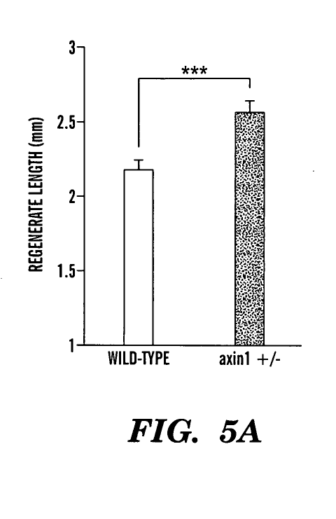

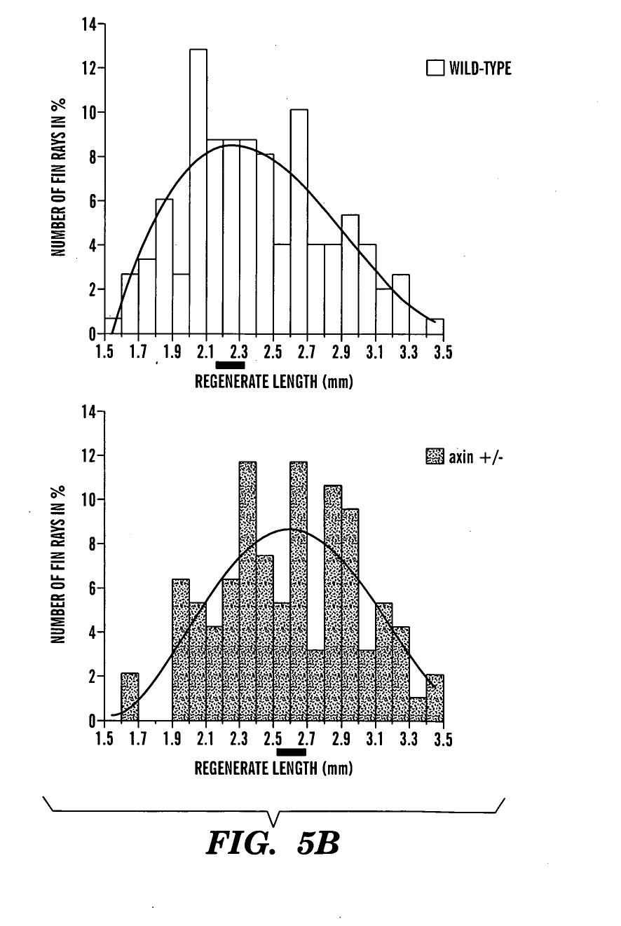

To test this, fish were used in which one copy of axin1/masterblind, an inhibitor of the Wnt/β-catenin signaling pathway, is mutated (Heisenberg et al., Genes Dev 15:1427-34, 2001) and asked whether axin1+/−fins regenerate more rapidly. To minimize effects of the genetic background, wild-type and axin1+/−fish were used that were siblings derived from a cross of a wild-type fish with an axin1 heterozygous carrier. We genotyped the fish, amputated fins of 12 wild-type and 9 axin1 heterozygous mutant fish, allowed them to regenerate for 7 days, photographed the fins, blinded the photographs, measured the length of the regenerate (from the amputation plane to the distal tip of the fin) in the second, third and fourth dorsal fin ray in each fish and calculated the average length of the regenerate of each fish. The same fish were re-amputated and re-measured twice after a two to three week recovery period. In the third experiment several additional fish were included. Intriguingly, in all three experiments the regenerates of axin1+/−fins were significantly longer (as determined by a Student's t-test) than those of wild-type siblings at 7 dpa (9.5%, 15.1% and 7.1% longer, respectively). FIG. 5A shows the average length of the regenerates in wild-type and axin1+/−fish as measured in the second experiment. Table 1 contains the data for all three experiments. To assess the error rate of measurement, we repeatedly measured the same pictures blinded by a colleague. The average error between repeated measurements was found to be 0.87%, and is thus insignificant compared to the difference measured between wild-type and axin1+/−fins. When we combine the measurements of individual wild-type fin rays from all three experiments and create a frequency histogram of the data, we find that the length of wild-type regenerates ranges from 1.48 to 3.36 mm, with the average length being 2.29 mm (FIG. 5B). The length of axin1 regenerates exhibits the same range as wild-type fins (1.52 to 3.33 mm). However, the distribution of the length of axin1 fin rays is shifted towards longer regenerates, with an average length of 2.49 mm (FIG. 5B). A Mann-Whitney test shows that the difference in fin ray length is highly significant (p=0.0003). In summary, these findings not only indicate that increased Wnt/β-catenin signaling results in faster regeneration, but also provide genetic evidence for an involvement of Wnt signaling in regenerative processes, which has not been heretofore addressed in any system.

FIGS. 4A, 4B, 4C, 4D, 4E, and 4F show β-catenin-dependent and β-catenin-independent Wnt signaling pathways have opposing roles in zebrafish fin regeneration. (A, D) Overexpression of Wnt8 in hsWnt8GFP transgenic fish induces the Wnt/β-catenin target gene axin2 in regenerating fins six hours after heat-shock at 3 dpa ([A], 3 of 4 fins), while overexpression of Wnt5b in hsWnt5bGFP transgenic fish represses axin2 expression ([D], n=4). Note that staining reactions were stopped as soon as a robust signal could be detected in most samples of one experimental group. Robust signal was first detected in hsWnt8GFP fins (vs. wild-type controls) in a short amount of time and reactions were stopped (A), while robust signal was first detected in wild-type controls (vs. hsWnt5GFP fins) after a longer staining reaction (D), thus accounting for the difference in wild-type signal between groups. (B, E) Cell proliferation in regenerating fins, as detected by BrdU incorporation and staining with an anti-phosphorylated histone 3 (PH3) antibody, is increased by overexpression of Wnt8 ([B], n=14), and repressed by overexpression of Wnt5b ([E], n=10). Fish were heat-shocked once at 66 hpa and fixed at 72 hpa. The percentage of BrdU or PH3 positive cells relative to the total number of cells in sections of regenerating fins is shown. Error bars represent the s.e.m. (C, F) While overexpression of Wnt8 for 10 days starting shortly before amputation has no obvious effect on overall length of the regenerate ([C], n=16), overexpression of Wnt5b completely inhibits regeneration ([F], n=16).

FIGS. 5A and 5B show fins regenerate faster in fish heterozygous for a loss-of-function mutation in axin1. (A) Average length of regenerating tail fins at 7 dpa is increased in fish heterozygous for an axin1 loss of function mutation (mbl^{tm013}) compared to wild-type siblings. Results of one representative experiment of three are shown. To determine the length of the regenerate for individual fish, the average length of the third, fourth and fifth dorsal regenerating fin ray was calculated. n=12 wild-type, 9 mbl heterozygous fish. Error bars represent the s.e.m. of the average regenerate lengths. p=0.0009 (one-tailed). (B) The number of fin rays (in percent of the total number counted) is blotted against the length of the regenerate (in 0.1 mm intervals) for wild-type (upper panel) and axin1 heterozygous fish (lower panel). The curves represent a 5th-order polynomial trendline. The average regenerate length is labeled by black bars at the x-axis. 148 fin rays were counted (combined results from 3 experiments) in 19 wild-type fish, 94 rays in 10 axin1+/−fish.

TABLE 1
mbl (axin1) heterozygous mutant fish regenerate their tail fins
faster than wild-type fish.

| | regenerate length 7 dpa (mm) | | |
|---|---|---|---|
| | wild-type | mbl +/− | different |
| exp 1 | 1.88 ± 0.047 | 2.08 ± 0.058 | YES |
| | n = 12 | n = 9 | p = 0.009 |
| exp2 | 2.17 ± 0.071 | 2.56 ± 0.077 | YES |
| | n = 12 | n = 9 | p = 0.00083 |
| exp 3 | 2.53 ± 0.058 | 2.73 ± 0.085 | YES |
| | n = 12 | n = 9 | p = 0.039 |

Tail fins of fish derived from a cross of a wild-type fish with a heterozygous axin1 mutant fish were amputated, fish genotyped and allowed to regenerate at 28° C. Each fish was photographed 7 days post amputation and photographs blinded before analysis. The length of the regenerate at the third, fourth and fifth dorsal fin ray was measured and the average length of the regenerate calculated for each fish. The average length calculated for each experimental group is printed in the table. The error represents the standard error of the average lengths of the individual fish. Student's t-test was used to test whether the average length of wild-type and axin1 mutant fish was significantly different. The p value (one-tailed) is printed in the table. The same fish were re-amputated twice and the measurements repeated twice. Note that in the first 2 experiments the average lengths of the regenerates are also significantly different using two-tailed t-tests.

To exclude that variations in the position of the amputation plane might have caused differences in regenerative speed, the exact position of the amputation plane was measured in each fish. The present study found that there was no significant difference in position of the amputation plane between wild-type and mbl fish.

### Example 5

### wnt5b Overexpression is Sufficient to Inhibit Fin Regeneration

As Wnts that can act through the Wnt/β-catenin pathway (wnt10a) and through 3-catenin-independent pathways (wnt5a, wnt5b/pipetail) are expressed during fin regeneration (FIG. 1) we next tested whether these distinct Wnt pathways might have different roles in fin regeneration. To this end, the effects of activation of Wnt/β-catenin signaling were compared to those produced by activation of β-catenin-independent Wnt signaling. To achieve this, a transgenic zebrafish line was generated carrying a heat-shock inducible Wnt5bGFP transgene (hsWnt5bGFP; FIG. 11). Wnt5b has been shown to activate β-catenin-independent signaling pathways in zebrafish embryos (Westfall et al., J Cell Biol 162:889-98, 2003). Accordingly, the present study found that heat-shocked hsWnt5bGFP embryos display the characteristic phenotypes associated with gain-of-function of β-catenin-independent Wnt pathways, namely defects in convergence-extension cell movements during gastrulation and somitogenesis (FIG. 11L, M).

Interestingly, while overactivation of Wnt/β-catenin signaling enhances regeneration, overexpression of Wnt5b represses regeneration. Heat-shock of hsWnt5bGFP transgenic fish for 10 days starting shortly before fin amputation completely inhibited fin regeneration (FIG. 4F). This is in marked contrast to overexpression of Wnt8, which had no obvious effect on overall fin morphology (FIG. 4C), but closely resembles the defects caused by inhibition of Wnt/β-catenin signaling via Dickkopf1 overexpression (FIG. 2B). Like overexpression of Dickkopf1, but in contrast to Wnt8, overexpression of Wnt5b significantly reduced proliferation of the blastema mesenchyme and overlying epithelium 6 hours after induction of the transgene as detected by BrdU incorporation and anti-phosphorylated histone 3 antibody staining (FIG. 4E). Thus, activation of Wnt5b inhibits fin regeneration.

While it is difficult to test which signaling pathways Wnt5b activates in the regenerating fin, the fact that it causes dramatically different effects than Wnt8, which signals via β-catenin, suggests that it likely acts through β-catenin-independent pathways. Since Wnt5b overexpression causes the same phenotypes as Wnt/β-catenin loss-of-function, and because β-catenin-independent Wnt signaling has been reported to be able to inhibit Wnt/β-catenin signaling in other systems (Weidinger, G. and Moon, R. T., J Cell Biol 162:753-5, 2003), we hypothesize that Wnt5b overexpression inhibits fin regeneration by repressing Wnt/β-catenin signaling. In support of this model, we found that Wnt5b overexpression abolished expression of the direct Wnt/β-catenin target gene axin2 6 h after heatshock at 3 dpa (FIG. 4D).

### Example 6

### wnt5b Loss-of-Function Augments Fin Regeneration

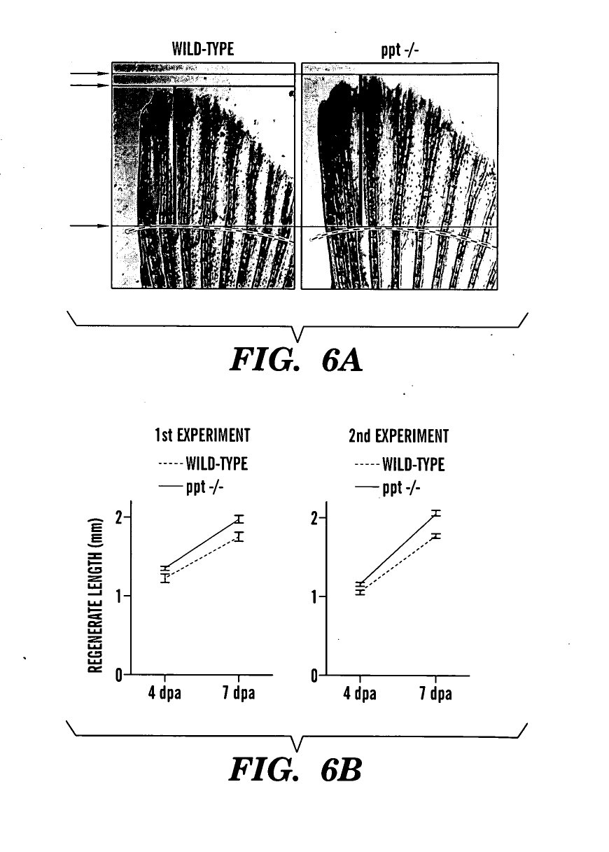

The present study next tested whether endogenous wnt5b acts as an essential modulator of fin regeneration. If non-canonical Wnt signaling activated by wnt5b inhibits regeneration in vivo, loss of wnt5b function in the regenerating fin might result in enhanced or faster regeneration. To test this prediction, homozygous adult wnt5b (pipetail) mutant fish were used. We amputated tail fins of wnt5b mutant and age- and size-matched wild-type fish of the same genetic background and measured the length of the regenerate at 4 and 7 dpa (FIG. 6A). In two independent sets of experiments using different fish, we found that wnt5b mutants had significantly longer regenerates than wild-types at both 4 and 7 dpa (FIG. 6A, B and Table 2). The difference in length between wild-type and wnt5b mutant regenerates increased between 4 and 7 dpa, showing that wnt5b mutant fins regenerate faster (FIG. 6B). To exclude that biases in the position of the amputation plane might have resulted in different speed of regeneration, we measured the position of the amputation plane in each fish and found that there was no significant difference between wild-type and wnt5b mutant fish. These data provide genetic evidence that wnt5b acts as a negative modulator of fin regeneration. Wnt5b mutant regenerating fins did not show any obvious patterning defects or indications of tumor formation or other signs of inappropriate growth (FIG. 6A), suggesting that wnt5b is only required to modulate the overall rate of regeneration.

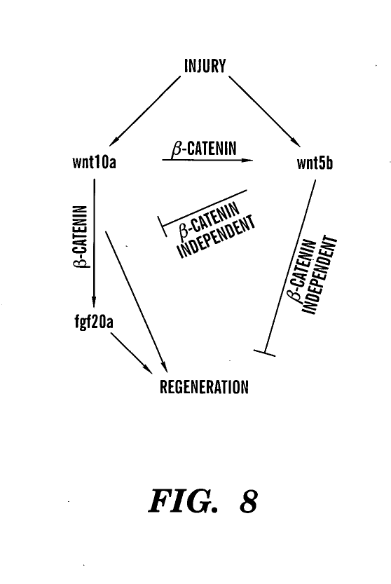

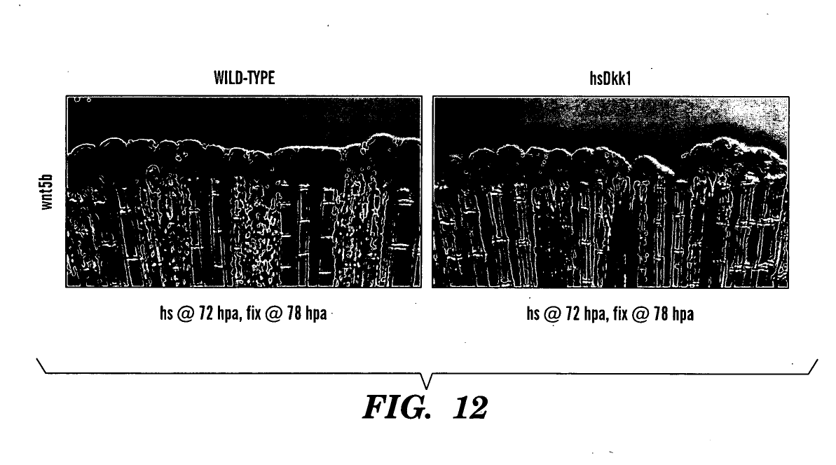

It is tempting to speculate that this antagonistic role of β-catenin-independent signaling activated by wnt5b (and possibly wnt5a) during fin regeneration represents a negative feedback mechanism that regulates levels and/or duration of Wnt/β-catenin signaling. If so, we would expect the expression of wnt5 paralogs to be regulated by Wnt/β-catenin signaling in the regenerating fin. Indeed, we found that wnt5b expression is downregulated in Dkk1-overexpressing fins 6 h after heatshock at 3 dpa (FIG. 12). Taken together, these data strongly suggest that β-catenin-independent Wnt signaling activated by wnt5b/pipetail and possibly wnt5a acts in a negative feedback loop to inhibit Wnt/β-catenin signaling during fin regeneration (FIG. 8).

FIGS. 6A and 6B show fins regenerate faster in wnt5b mutant fish. (A) Dorsal half of regenerating tail fins of wild-type and wnt5b (ppt) homozygous mutant fish at 7 dpa. The amputation plane is indicated by a dashed red line, the length of the third fin ray by colored bars. Note that the regenerate is longer in ppt than in wild-type fish. (B) The average length of the regenerate of wild-type and ppt mutant fish at 4 and 7 dpa in 2 independent experiments. To determine the length of the regenerate for individual fish, the average length of the third, fourth and fifth dorsal regenerating fin ray was calculated. Experiment 1: n=14 wild-type, 12 ppt fish. Experiment 2: n=12 wild-type, 11 ppt fish. Error bars represent the s.e.m. of the average regenerate lengths. See Table 2 for p values. Note that absolute fins lengths cannot be compared between experiments, since water temperatures and thus regenerative speed and exact times of photography varied between experiments.

TABLE 2
ppt (wnt5b) homozygous mutant fish regenerate their tail fins
faster than wild-type fish.

| | regenerate length 4 dpa (mm) | | | regenerate length 7 dpa (mm) | | |
|---|---|---|---|---|---|---|
| | wild-type | ppt −/− | different? | wild-type | ppt −/− | different? |
| exp 1 | 1.23 ± 0.05 | 1.35 ± 0.03 | YES (Mann Whitney) | 1.76 ± 0.05 | 1.76 ± 0.05 | YES (Mann Whitney) |
| | n = 14 | n = 12 | P = 0.022 | n = 14 | n = 14 | P = 0.016 |
| exp 2 | 1.06 ± 0.02 | 1.16 ± 0.02 | YES (t test) | 1.76 ± 0.05 | 1.76 ± 0.05 | YES (t test) |
| | n = 12 | n = 11 | P = 0.0018 | n = 14 | n = 14 | P = 0.00000006 |

Tail fins of ppt homozygous mutant fish and age- and size-matched wildtype fish of the same genetic background were amputated and fish were allowed to regenerate at 28° C. Each fish was photographed at 4 and 7 days post amputation and photographs were blinded before analysis. The length of the regenerate at the third, fourth and fifth dorsal fin ray was measured and the average length of the regenerate calculated for each fish. The average length calculated for each experimental group is printed in the table. The error represents the standard error of the average lengths of the individual fish. Normal distribution of the data was tested using a Shapiro-Wilk W Test. If data were normally distributed a student's t-test was used to test whether the average length of wild-type and ppt mutant fish was significantly different, otherwise a Mann Whitney tested was used. The p value (one-tailed for t-tests) is printed in the table.

To exclude that variations in the position of the amputation plane might have caused differences in regenerative speed, the exact position of the amputation plane was measured in each fish. We found that there was no significant difference in position of the amputation plane between wild-type and ppt fish.

FIG. 8 shows a model of signaling events regulating zebrafish fin regeneration. We propose that injury of the tail fin activates as yet unknown signals that result in upregulation of wnt10a and wnt5b. wnt5b expression is also regulated by Wnt/β-catenin signaling activated by wnt10a. Wnt10a activates a β-catenin-dependent signaling pathway that positively regulates fgf20a expression, which has been shown to be required for blastema formation and subsequent regeneration (Whitehead et al., 2005). In addition to its role in regulating fgf20a expression, Wnt/β-catenin signaling might also regulate other genes that are required for blastema formation and proliferation (gray arrow). We propose that wnt5b employs a β-catenin-independent signaling pathway that antagonizes Wnt/β-catenin signaling. However, we cannot exclude that such β-catenin-independent pathways also inhibit regeneration without impairing Wnt/β-catenin signaling (gray arrow). Arrows do not imply direct events.

### Example 7

### Wnt/β-Catenin Signaling Regulates FGF Signaling During Fin Regeneration

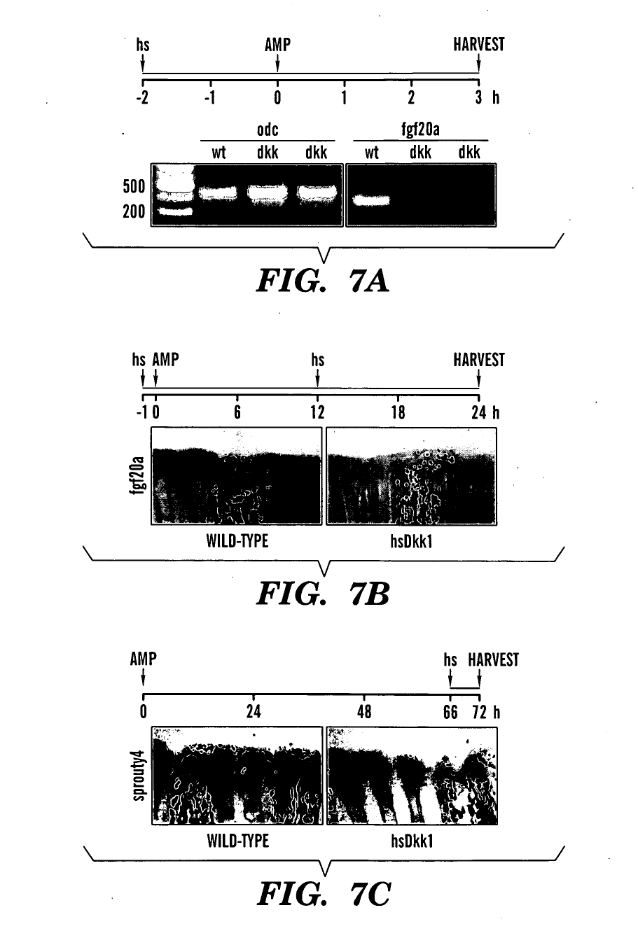

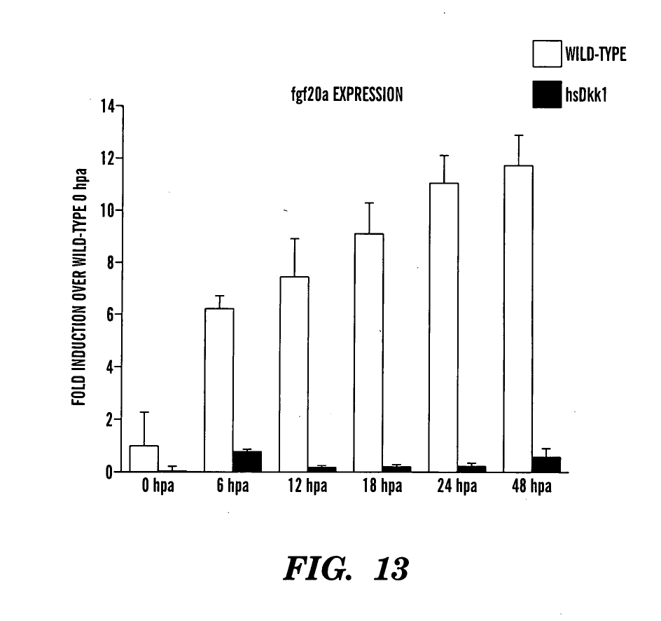

FGF signaling has been shown to be required for regeneration of amphibian and fish appendages (Lee et al., Development 132:5173-83, 2005; Poss et al., Dev Biol 222:347-58, 2000; Yokoyama et al., Dev Biol 233:72-9, 2001) and recently fgf20a was found to be induced early during zebrafish fin regeneration and to be required for blastema formation (Whitehead et al., Science 310:1957-60, 2005). Similarly, we observe that wnt10a is induced very early in regenerating fins and that Wnt/β-catenin signaling is essential for formation of the blastema. Therefore, to gain more mechanistic insight into the role of Wnt/β-catenin signaling in fin regeneration, we investigated whether Wnt/β-catenin signaling regulates FGF signaling during regeneration. Strikingly, the present study found that levels of fgf20a transcripts are suppressed three hours after amputation in Dkk1-overexpressing fins (FIG. 7A), and that fgf20a expression is still not detectable in hsDkk1GFP fins at 24 hpa (FIG. 7B). Quantitative PCR shows that induction of Dkk1 two hours prior to amputation results in a severe downregulation of the baseline of fgf20a expression at the time of amputation and in suppression of fgf20a upregulation during the first 48 h of regeneration (FIG. 13). These findings show that Wnt/β-catenin signaling is required for initiation of fgf20a expression during regeneration. The fast response and the repression of basic fgf20a levels in hsDkk1 fins indicate that fgf20a downregulation is not an indirect consequence of a failure of these fins to regenerate, but likely reflects a more direct regulation of fgf20a expression by Wnt/β-catenin signaling.

Furthermore, in fins that have been allowed to regenerate normally for 72 h, a single pulse of Dkk1 expression quickly results in repression of sprouty4, a FGF target gene (Lee et al., Development 132:5173-83, 2005) (FIG. 7C). We conclude that Wnt/β-catenin signaling is also required for maintenance of FGF signaling. These findings indicate that Wnt/β-catenin signaling acts upstream of FGF signaling during regeneration, placing Wnt/β-catenin signaling at the top of the hierarchy of signaling pathways known to be required for epimorphic regeneration (FIG. 8).

FIGS. 7A, 7B, and 7C show Wnt/β-catenin regulates FGF signaling during fin regeneration. (A) fgf20a expression as detected by semi-quantitative RT-PCR is greatly reduced in Dkk1-overexpressing fins at 3 hpa. Wild-type and hsDkk1GFP transgenic fish were treated according to the schematic (hs=heat-shock, amp=amputation, green line indicates inhibition of Wnt/β-catenin signaling), and RNA was harvested from the tissue adjacent to the amputation plane of nine wild-type and two groups of nine hsDkk1GFP transgenic fins. Ode amplification serves as loading control. The experiment was repeated four times using two sets of biological samples, representative results are shown. (B) fgf20a expression is greatly reduced as detected by in situ hybridization in hsDkk1GFP transgenic fins (5 of 6 fins) at 24 hpa compared to wild-type fins. (C) sprouty4 expression is greatly reduced 6 h after heat-shock in hsDkk1GFP fins (n=3) at 72 hpa.

### Example 8

### Role for Wnt/β-Catenin Signaling in Epimophic Regeneration of the Zebrafish Tail Fin

The present findings further our understanding of the molecular events that initiate regenerative processes by demonstrating a critical role for Wnt/β-catenin signaling in epimophic regeneration of the zebrafish tail fin, and an antagonistic role for β-catenin-independent Wnt signaling. Based on the present results and those of others, the following model was proposed for signaling pathways regulating zebrafish fin regeneration (FIG. 8):

Injury of the fin activates signal(s) that rapidly induce expression of wnt10a and possibly other Wnt ligands that activate the β-catenin signaling pathway. The nature of these signals and whether they directly or indirectly regulate expression of Wnt ligands is unknown. One potential candidate that might indirectly activate Wnt expression is thrombin, which is activated by the wound healing response and has been shown to be involved in regeneration of newt lens and limb myotubes (Imokawa, Y. and Brockes, J. P. Curr Biol 13:877-81, 2003; Imokawa et al., Philos Trans R Soc Lond B Biol Sci 359:765-76, 2004; Tanaka et al., Curr Biol 9:792-9, 1999). Wnt10a activates the β-catenin signaling pathway, which directly or indirectly activates expression of fgf20a, which in turn activates (directly or indirectly) the events resulting in blastema formation and thus regeneration (Whitehead et al., Science 310:1957-60, 2005). While we have not tested whether wnt10a activates fgf20a expression directly in the regenerating fin, it is intriguing that fgf20a has been found to be a direct target of Wnt/β-catenin signaling in cultured human cells (Chamorro et al., Embo J 24:73-84, 2005). Since we have not tested whether Wnt/β-catenin signaling acts solely through fgf20a to regulate blastema formation, we cannot exclude the possibility that β-catenin signaling also controls regeneration in parallel to FGF signaling (gray arrow in FIG. 8).

The same injury-activated signal(s) that regulate wnt10a expression might also activate expression of wnt5a and wnt5b and potentially other Wnt ligands that activate β-catenin-independent signaling. We postulate that these signaling pathways modulate regeneration by negatively regulating Wnt/β-catenin signaling. However, we cannot exclude the possibility that β-catenin-independent Wnt signaling represses regeneration independently of its antagonistic role on β-catenin signaling as well (gray arrow in FIG. 8). Because we find that expression of wnt5b is regulated by Wnt/β-catenin signaling, we hypothesize that these separate Wnt pathways establish a negative feedback loop whose function might be to ensure proper levels, duration or location of β-catenin signaling in the regenerating fin.

In addition to its role in blastema formation, FGF signaling appears to be absolutely required for the regenerative outgrowth of the fin, since drugs that block FGF signaling can inhibit fin regeneration during this phase (Poss et al., Dev Biol 222:347-58, 2000). Our experiments indicate that Wnt/β-catenin signaling is also required for regenerative outgrowth. However, overexpression of Dickkopf1 does not cause a complete inhibition of outgrowth. It is possible that the expression levels of Dkk1 are not sufficient to completely block β-catenin signaling during this regenerative phase. Alternatively, other signals that act partially redundant to Wnt/β-catenin signaling might compensate for the loss of Wnt signaling. We have found that Wnt/β-catenin signaling regulates FGF signaling during regenerative outgrowth, thus it appears likely that β-catenin signaling acts through FGF signaling in this phase of regeneration as well.

Elucidation of the exact cell biological role of both Wnt/β-catenin and FGF signaling in blastema formation awaits further experiments. While regeneration of the zebrafish tail fin occurs in similar steps as salamander limb regeneration, blastema formation by de-differentiation of differentiated cells has so far only been reported in salamanders. Interestingly, a recent report has shown that resident muscle stem cells are activated during salamander limb regeneration and that progeny of these cells take part in formation of the blastema (Morrison et al., J Cell Biol 172:433-40, 2006). It is likely that the relative contribution of de-differentiation and resident stem cell activation to formation of progenitor cells during regeneration varies between organs and organisms, with amphibian limbs likely representing one end of the spectrum where de-differentiation is prominent, and processes like mammalian muscle or bone regeneration being driven only by activation of resident stem cells. Whether Wnt/β-catenin and FGF signaling regulate de-differentiation or stem cell activation or both in blastema formation is at present unclear. Interestingly, Wnt/β-catenin signaling has been shown to be important for regeneration or repair of systems that most likely rely largely or solely on activation of resident stem cells. Inhibition of Wnt/β-catenin signaling reduces proliferation of CD45+ resident stem cells in mammalian muscle regeneration (Polesskaya et al., Cell 113:841-52, 2003) and inhibits proliferation of osteoblasts, which drive bone repair, in culture (Zhong et al., Bone 39:5-16, 2006). Wnt/β-catenin signaling has also been reported to be active during regeneration of deer antlers and to be required for survival of antler bone progenitor cells in culture (Mount et al., Dev Dyn 235:1390-9, 2006). Very recently, Hayashi et al. have shown that Wnt/β-catenin signaling is necessary and sufficient for regeneration of newt lenses in culture (Hayashi et al., 2006). More specifically, Wnt signaling appears to regulate the second step of regeneration, in which, subsequent to proliferation of the iris pigmented epithelium and activation of early lens genes in the whole iris, only the dorsal iris continues to develop (Hayashi et al., Mech Dev 123(11):793-800, 2006). Thus, together with our results showing that β-catenin signaling is required for fin regeneration and our data that β-catenin signaling is activated during mouse liver and zebrafish heart regeneration, evidence is beginning to emerge that Wnt/β-catenin signaling might play central roles in many regenerative processes. However, the specific function of Wnt signaling during regeneration of different organs is most likely different. For example, in the newt lens Wnt signaling is only activated after the initial phase of proliferation and gene expression and is required for the second step of regeneration. In contrast, we have shown that Wnt signaling regulates gene expression very early during fin regeneration and that it is required for the early events of blastema formation.

At present, a better understanding of the role of Wnt/β-catenin and FGF signaling in de-differentiation and/or stem cell activation during epimorphic regeneration is hampered by the fact that our insights into signaling events that regulate epimorphic regeneration come mainly from systems like zebrafish, where de-differentiation has not been reported. Thus, further insights into the role of these pathways awaits a better characterization of the cell biological events of blastema formation in zebrafish or the development of tools that facilitate genetic and other in vivo functional studies in salamanders.

While our study demonstrates an important role for Wnt/β-catenin signaling during regeneration, it also adds to our knowledge about the functions of β-catenin-independent Wnt signaling in adults. In vertebrates β-catenin-independent Wnt signaling is well established to be required for cell polarity and cell movements during gastrulation, and has also been implicated in endoderm cell migration, pancreas cell migration, migration of neurons and organization of hair cell polarity in the inner ear (Bingham et al., Dev Biol 242:149-60, 2002; Carreira-Barbosa et al., Development 130:4037-46, 2003; Curtin et al., Curr Biol 13:1129-33, 2003; Jessen et al., Nat Cell Biol 4:610-5, 2002; Kim et al., BMC Biol 3:23, 2005; Matsui et al., Genes Dev 19:164-75, 2005; Wada et al., Development 132:2273-85, 2005). It is less clear whether β-catenin-independent Wnt signaling plays roles in cell fate determination. Interestingly, however, it has been shown that β-catenin-independent Wnt signaling can inhibit Wnt/β-catenin signaling and thus can, indirectly, regulate cell fate. For example, overexpression of β-catenin-independent Wnt ligands in Xenopus blocks the ability of “canonical” Wnt ligands to activate β-catenin signaling and to induce a secondary body axis. Genetic evidence for the existence of such opposing roles of β-catenin-independent Wnt signaling on Wnt/β-catenin signaling comes from zebrafish, where maternal loss of wnt5b has been reported to result in ectopic β-catenin signaling and consequent increase in dorsal cell fates (Weidinger, G. and Moon, R. T., J Cell Biol 162:753-5, 2003; Westfall et al., J Cell Biol 162:889-98, 2003). Furthermore, loss of wnt5a in mouse limb buds likewise results in ectopic β-catenin, causing defective chondrocyte differentiation (Topol et al., J Cell Biol 162:899-908, 2003). We propose that β-catenin-independent Wnt signaling, activated by wnt5a and wnt5b plays a similar antagonistic role in fin regeneration. Our finding that wnt5b expression appears to be regulated by β-catenin signaling suggests the existence of a negative feedback loop. Such a loop represents a new mechanism for regulation of β-catenin signaling, that, to our knowledge, has not been described before. It will be interesting to see whether transcriptional activation of Wnt ligands that activate antagonistic β-catenin-independent pathways is a more widespread regulatory mechanism employed by organisms to keep β-catenin signaling in check.

Taken together, our findings add to our mechanistic insight into the regulation of regeneration by demonstrating separate and opposing roles for β-catenin dependent and independent signaling pathways during fin regeneration. Furthermore, while regeneration of the mammalian liver and the zebrafish heart employ different cellular mechanisms than regeneration of the zebrafish fin or amphibian limbs (with only the latter two involving formation of a blastema), it is intriguing that Wnt/β-catenin signaling is upregulated during regeneration of all three organs. While being beyond the scope of this study, it will be very interesting to test what role Wnt signaling plays in regeneration of these organs. It is conceivable that our findings will prove to be important for the goals of regenerative medicine, since modulation of Wnt signaling pathways might augment regeneration of human tissues.

FIGS. 9A, 9B, and 9C show Wnt/β-catenin signaling is upregulated in regenerating zebrafish heart and mouse liver. (A) Wnt/β-catenin reporter (TOPdGFP) activity (detected by in situ hybridization for GFP RNA, blue staining) is upregulated in regenerating zebrafish hearts (n=4) adjacent to the amputation plane (arrowheads) at 3 dpa (*=blood clot). Control is a sham-operated TOPdGFP heart at 3 d post sham. TOPdGFP is also expressed adjacent to the amputation plane at 7 dpa (n=3) and 14 dpa (n=3) (data not shown). (B) Wnt/β-catenin reporter (TOPGal (DasGupta and Fuchs, 1999)) activity (detected by staining for β-galactosidase activity in blue) is upregulated in periportal hepatocytes in regenerating mouse liver (n=8), 48 hours post partial hepatectomy (see FIG. 9 for quantitation of β-galactosidase activity). (C) Quantification of TOPGal activity in regenerating mouse liver. β-galactosidase activity was measured in control lobes (resected lobes removed at the time of partial hepatectomy) and regenerating lobes, collected 48 hpa. Control lobes were compared to regenerating lobes harvested from the same animal. Asterisks indicate a statistically significant difference between experimental groups (n=8, p<0.01, Paired Student's T-Test). Error bars represent the s.e.m.

FIGS. 10A and 10B show the previously described zebrafish wnt5 homolog pipetail (ppt) is the ortholog of wnt5b, while a newly cloned homolog likely represents the zebrafish ortholog of wnt5a. (A) Multiple sequence alignment of several vertebrate Wnt5 paralogs. The sequence described here and ppt are depicted in red. (B) Phylogenetic tree analysis of Wnt5 protein sequences using the PAUP software. Zebrafish Ppt clearly groups with Wnt5b paralogs of other vertebrates. The newly described sequence, together with predicted proteins from Fugu and Tetraodon does not clearly group with Wnt5a paralogs of other vertebrates, but the resolution of the tree is too low to exclude that they belong in the Wnt5a clade. Since ppt clearly represents zebrafish wnt5b and since there is no evidence for the existence of a third wnt5 paralog in zebrafish, Fugu or Tetraodon, we suggest that the newly described sequence represents the zebrafish ortholog of wnt5a.

Multiple sequence alignment of Wnt5 homologs was built using Clustalw. The non-conserved N-terminus (signal sequence) was excluded from the alignment, as were some exons from predicted Wnt5 homologs that are non-conserved. Settings used in PAUP to build the tree were: outgroup to wnt5\ciona, 10000 bootstrap replicates, neighbor joining, 50% majority rule consensus.

Accession numbers: wnt5a\mouse=NP_{—}033550 (SEQ ID NO: 3), wnt5a\rat=NP_{—}072153, wnt5a\dog=XP_{—}541837, wnt5a\human=NP_{—}003383 (SEQ ID NO: 2), wnt5a\cow=XP_{—}878444, wnt5a\chick=BAA75242 (SEQ ID NO: 5), wnt5a\Xenopus=P31286 (SEQ ID NO: 4), wnt5a\axolotl=WN5A_AMBME (SEQ ID NO: 6), wnt5b\human=NP_{—}116031 (SEQ ID NO: 8), wnt5b\chimpanzee=XP_{—}522589, wnt5b\mouse=NP_{—}033551 (SEQ ID NO: 9), wnt5b\rat=XP_{—}342748, wnt5b\cow=XP_{—}584724, wnt5b\axolotl=WN5B_AMBME (SEQ ID NO: 11), wnt5c\Xenopus=WN5C_XENLA (SEQ ID NO: 10), wnt5b\fugu=ENSEMBL NEWSINFRUP00000152673, wnt5b\tetraodon=ENSEMBL GSTENT00034446001, wnt5\zebrafish\ppt=NP_{—}571012 (SEQ ID NO: 7), wnt5a\fugu=ENSEMBL NEWSINFRUP00000133995, wnt5a\tetraodon=ENSEMBL GSTENT00027963001, wnt5\zfish\NEW=XXXX (SEQ ID NO: 1), wnt5\ciona=NP_{—}001027951.

FIG. 11A through 11M show establishment of heat-shock inducible Dickkopf1GFP and heat-shock inducible Wnt5bGFP transgenic zebrafish lines. (A) Maps of the transgenes used. mmGPF5 (Siemering et al., Curr Biol 6:1653-63, 1996) was fused to the C-terminus of zebrafish dkk1 (Genbank accession #AB023488). Upon injection into zebrafish embryos, RNA encoding this fusion protein was found to cause posterior truncations and increased size of eyes and forebrain at similar doses as the wild-type dkk1 RNA (data not shown). Likewise, mmGFP5 was fused to the C-terminus of zebrafish wnt5b/pipetail (see FIG. 10 for nomenclature, Genbank accession #DRU51268). Injection of RNA coding for this fusion protein into early zebrafish embryos caused similar gastrulation defects as RNA coding for the wild-type Wnt5b protein, but the fusion protein appeared to be significantly less active (data not shown). Both fusion proteins were cloned downstream of a 1.5 kb fragment of the zebrafish hsp70-4 promoter (Halloran et al., Development 127:1953-60, 2000) and upstream of the SV40 polyadenylation signal of the vector pCS2+. An I-SceI meganuclease restriction site was inserted 5′ of the transgene. (B) Strategy used to establish and characterize transgenic fish. Supercoiled plasmid DNA containing the transgenes was injected together with I-SceI meganuclease (Thermes et al., Mech Dev 118:91-8, 2002) into 1-cell stage embryos to create mosaic G0 founder fish. Founders that transmitted a functional transgene through their germline were identified by crossing them to wild-type fish, heat-shocking the resulting F1 embryos and screening them for GFP expression. Transgenic F1 embryos were found to be viable when heat-shocked at 24 hpf or later and therefore could be raised to adulthood. To establish transgenic lines, identified heterozygous F1 fish were crossed to wild-type fish and the F2 generation raised. For most experiments on adult fish, wild-type siblings from such crosses served as controls. When siblings could not be used, age-matched wild-types served as controls. (C-D) Heat-shock of heterozygous hsDkk1GFP transgenic embryos at 24 hpf for 2.5 h results in ubiquitous expression of the Dkk1GFP fusion protein. (E-F) Heat-shock of heterozygous hsDKK1GFP transgenic adult fish for 1 h causes expression of the fusion protein in amputated tail fins. (G-I) Heat-shock of heterozygous hsDkk1GFP transgenic embryos for 2 h during gastrulation (starting at shield stage, 6 hpf) causes severe defects in anteroposterior patterning that closely resemble those caused by knock down of both ORFs of wnt8 using translation blocking morpholinos. Heterozygous carriers of the transgene were crossed to wild-type fish, and the resulting clutch of embryos heat-shocked at 6 hpf for 2 h. Wild-type and transgenic embryos were identified by GFP fluorescence, sorted and photographed at 24 hpf. (J-K) Overexpression of Dkk1GFP in embryos doubly transgenic for hsDKK1 GFP and the reporter TOPdGFP is sufficient to suppress β-catenin-dependent expression of the TOPdGFP transgene. Heterozygous carriers of the hsDkk1GFP transgene were crossed to homozygous carriers of the TOPdGFP transgene. The resulting clutch of embryos was heat-shocked at 9 hpf for 1 h, embryos fixed at 12 hpf and processed for two-color in situ hybridization with an EGFP probe detecting the TOPdGFP transgene in blue and a mmGFP probe detecting the dkk1GFP transgene in brown. The pictures show the head region of flat-mounted embryos, anterior left. Expression of TOPdGFP at this stage is primarily in the tectum and hindbrain. (L-M) Overexpression of Wnt5bGFP in embryos heterozygous for the hsWnt5bGFP transgene during gastrulation causes phenotypes typical for activation of β-catenin-independent Wnt signaling pathways, namely convergent-extension defects, resulting in short embryos with compressed somites and wavy notochord. Heterozygous carriers for the hsWnt5GFP transgene were crossed to wild-type fish, the resulting embryos heat-shocked at 6 hpf for 2 h, sorted into wild-type and transgenic carriers by GFP fluorescence and photographed at 24 hpf.

FIG. 12 shows wnt5b expression during fin regeneration is regulated by Wnt/β-catenin signaling. Fins of hsDkk1GFP transgenic and wild-type fish were amputated, fish heatshocked once at 72 hpa and fins fixed 6 h later. wnt5b expression is downregulated in Dkk1-expressing fins (n=10).

FIG. 13 shows heat-shock induced expression of Dkk1 represses fgf20a expression during fin regeneration. Wild-type and hsDkk1GFP transgenic zebrafish were heatshocked 2 h prior to caudal fin amputation, and every 12 h during the course of the experiment to ensure continual Dkk1 expression. Total RNA was isolated from the distal tip of the regenerating fins of 8 fish at each time point indicated and fgf20a expression levels detected by quantitative RT-PCR. fgf20a transcript levels were normalized to levels of 18S ribosomal RNA. The values plotted show fold-induction over wild-type and error bars represent the confidence interval of the ratios between 2 repeated PCRs of the same sample, p<0.001. Note that the base-line expression of fgf20a at the time of amputation and the previously reported induction of fgf20a expression during fin regeneration are suppressed upon the expression of Dkk1.

### Example 9

### Methods

Zebrafish surgeries. Zebrafish ˜6-+12 months of age were used for all studies. Zebrafish heart and fin amputations were performed as previously described (Poss et al., Dev Dyn 219:282-6, 2000; Raya et al., Cloning Stem Cells 6:345-51, 2004). After zebrafish fin and heart amputations, fish were returned to 28° C.-30° C. water.

Partial hepatectomy in TOPGAL mice. TOPGAL mice have been described previously (DasGupta, R. and Fuchs, E., Development 126:4557-68, 1999) (a gift from E. Fuchs, Rockefeller University, NY). We performed ⅔ partial hepatectomy (Campbell et al., Immunol 176:2522-8, 2006) and sham laparotomy on 8-11 week old male TOPGAL mice in the morning after a night of fasting. Resected lobes were collected and served as control tissue for subsequent experiments; remnant livers were harvested 48 hours later. β-galactosidase activity was determined in whole liver lysates per manufacturer instructions (Promega, Madison, Wis.), and normalized to total protein concentration as determined by the Bradford assay (Bio-rad, Hercules, Calif.). X-gal staining was performed on glutaraldehyde-fixed 5 μm frozen liver sections per manufacturer instructions (Gold Bio Technology, St. Louis, Mo.).

Cloning of zebrafish wnt5a. Zebrafish genomic sequence was searched for sequences homologous to the previously known zebrafish Wnt5 ortholog, pipetail (ppt). A sequence distinct from ppt was identified and a partial cDNA coding for this wnt5 paralog cloned by RT-PCR from a mixture of RNA isolated at different stages of embryonic development. The 5′ end of the cDNA was defined by RACE and by homology to EST 052-H12-2. The very 3′ end of the open reading frame and a putative 3′UTR were predicted from genomic sequences, but have not been experimentally verified. BLAST searches, multiple sequence alignments of the predicted protein sequence with Wnt5 paralogs from other species and phylogenetic analysis using the PAUP program support the conclusion that the previously described zebrafish wnt5 paralog ppt is the zebrafish ortholog of wnt5b, while the newly cloned paralog described here is most likely the ortholog of wnt5a (see FIG. 10). We have thus deposited the new sequence as wnt5a in genbank (accession number DQ465921) and suggest that wnt5/ppt should be renamed wnt5b.

In situ hybridization. Whole-mount in situ hybridization was performed on amputated fins and hearts as described previously (Poss et al., Dev Dyn 219:282-6, 2000). For Digoxigenin-labeled probe synthesis, published templates were used, except for wnt5a cDNA, which was cloned by RT-PCR from RNA isolated from embryos at different stages of development. When assaying for differences in expression, development of the staining reaction was monitored carefully and fins or hearts of the same comparative groups were stopped at exactly the same time. Cryosectioning of the fins was performed as described previously (Poss et al., Dev Biol 222:347-58, 2000).

Heat-shock inducible transgenic zebrafish lines. Establishment of the hsDkk1 GFP and hsWnt5bGFP lines is described in the legend of FIG. 11. Heat-shocks for these lines and the hsΔTCFGFP and hsWnt8GFP lines were performed twice daily by transferring fish from 28° C.-30° C. water to water preheated to 38° C. and subsequent incubation in an air incubator at 39° C. for one hour.

Tissue sectioning and histology. Hematoxylin staining and histology were performed as previously described (Poss et al., Science 298:2188-90, 2002). Cryostat sections of 20 μM were used in these analyses.

BrdU incorporation and mitosis analysis. BrdU incorporation and mitosis analysis were performed as previously described (Nechiporuk, A. and Keating, M. T., Development 129:2607-17, 2002). All BrdU incorporations were performed for the final 1-2 hours of the experiment. Sections were rinsed three times in PBS, then incubated in 2N HCl for 30 min at 37° C. Sections were then briefly rinsed in PBS three times and incubated in blocking solution (PBS/1% Triton X-100/0.25% BSA) for >1 hour. Slides were incubated in mouse anti-BrdU (1:200; Sigma, St. Louis, Mo.) and rabbit anti-phosphorylated histone 3 (PH3; 1:200, Upstate Biotechnology, Charlottesville, Va.) antibodies overnight at room temperature. Slides were washed all day with multiple changes of PBS the following day and then incubated in secondary antibodies (goat anti-mouse Alexa-fluor-546; goat anti-rabbit Alexa-fluor-488, Molecular Probes) for 1-2 hours at room temperature. Slides were rinsed three times in PBS (20 min each) and mounted with Dapi mounting media and coverslipped. DAPI stained nuclei, BrdU positive cells and PH3 positive cells were counted from 3-6 sections per fin from 3 fins per wild-type or transgenic sample. n=number of blastemas counted per experiment.

Fin length measurements in axin1 and wnt5b mutant fish. Heterozygous carriers of the axin1 mutation mbl^{tm013} and wild-type sibling fish were identified by genotyping using allele-specific PCR. ppt^{ta98} (wnt5b) homozygous mutant embryos were identified by their phenotype in an incross of heterozygous carriers. Since some homozygous embryos survive, identified embryos could be raised to adulthood. The length of individual regenerating fin rays (from the amputation plane to the distal tip of the fin) was measured using IMAGE J software (NIH, http://rsb.info.nih.gov/ij/).

Semi-quantitative and quantitative RT-PCR. Total RNA was isolated from zebrafish fin regenerates into TRIZOL (Invitrogen). RNA was extracted according to the manufacturer's protocol, DNAse digested and subsequently purified with the Qiagen RNeasy kit. Equal amounts of total RNA from different samples was reverse transcribed with Thermoscript reverse-transcriptase (Invitrogen) using oligo (dT) and random hexamer primers. For semi-quantitative PCR, amplification of ornitine decarboxylase (odc) was used as loading control. fgf20a (primers GCAGATTTGGTATATTGGAATTCAT (SEQ ID NO: 12) and CTAGAACATCCTTGTAAAGCTCAGG (SEQ ID NO: 13)) and odc (primers ACTTTGACTTCGCCTTCCTG (SEQ ID NO: 14) and CACCTTCATGAGCTCCACCT (SEQ ID NO: 15)) PCR products were detected on ethidium bromide stained agarose gels. Quantitative PCR was performed using a Roche Lightcycler and the SYBR green labeling system. wnt10a was amplified using ATTCACTCCAGGATGAGACTTCATA (SEQ ID NO: 16) and GTTTCTGTTGTGGGCTTTGATTAG (SEQ ID NO: 17) primers. wnt10a expression levels were normalized to β-actin (primers GGTATGGGACAGAAAGACAG (SEQ ID NO: 18) and AGAGTCCATCACGATACCAG (SEQ ID NO: 19)) or 18S rRNA (primers CGCTATTGGAGCTGGAATTACC (SEQ ID NO: 20) and GAAACGGCTACCACATCCAA (SEQ ID NO: 21)) levels. Primers used for quantitative PCR of fgf20a were CAGCTTCTCTCACGGCTTGG (SEQ ID NO: 22) and AAAGCTCAGGAACTCGCTCTG (SEQ ID NO: 23).

What is claimed:

1. A method for increasing cell or tissue regeneration in a vertebrate subject comprising,
administering one or more Wnt/β-catenin signal-promoting agents to the vertebrate subject,
increasing in vivo a stem cell, progenitor cell, or differentiated cell population in the vertebrate subject compared to the stem cell, progenitor cell, or differentiated cell population in the vertebrate subject before treatment, to increase cell or tissue regeneration in the vertebrate subject.

2. The method of claim 1, wherein the Wnt/β-catenin signal-promoting agent is an agonist of one or more of Wnt1, Wnt2, Wnt2b/13, Wnt3, Wnt3a, Wnt4, Wnt6, Wnt7a, Wnt7b, Wnt7c, Wnt8, Wnt8a, Wnt8b, Wnt8c, Wnt10a, Wnt10b, Wnt11, Wnt14, Wnt15, Wnt16, or fgf20a.

3. The method of claim 2, wherein the Wnt/β-catenin signal-promoting agent is an agonist of Wnt10a.

4. The method of claim 2, wherein the Wnt/β-catenin signal-promoting agent is an agonist of Wnt3a or Wnt8.

5. The method of claim 1 wherein said cell or tissue regeneration occurs in bone, chondrocytes/cartilage, muscle, skeletal muscle, cardiac muscle, pancreatic cells, endothelial cells, vascular endothelial cells, adipose cells, liver, skin, connective tissue, hematopoietic stem cells, neonatal cells, umbilical cord blood cells, fetal liver cells, adult cells, bone marrow cells, peripheral blood cells, erythroid cells, granulocyte cells, macrophage cells, granulocyte-macrophage cells, B cells, T cells, multipotent mixed lineage colony types, embryonic stem cells, mesenchymal progenitor/stem cells, mesodermal progenitor/stem cells, neural progenitor/stem cells, or nerve cells.

6. The method of claim 1 wherein the vertebrate is mammalian, avian, reptilian, amphibian, osteichthyes, or chondrichthyes.

7. The method of claim 1 wherein the Wnt/β-catenin signal-promoting agent is a glycogen synthase kinase (GSK) inhibitor.

8. The method of claim 7 wherein the GSK inhibitor is a GSK-3 inhibitor or a GSK-3β inhibitor.

9. The method of claim 1, wherein the Wnt/β-catenin signal-promoting agent is a polypeptide, peptide mimetic, nucleic acid, small chemical molecule, antisense oligonucleotide, ribozyme, RNAi construct, siRNA, shRNA, or antibody.

10. The method of claim 9, wherein the Wnt/β-catenin signal-promoting agent is a polypeptide or peptide mimetic.

11. The method of claim 10, wherein the Wnt signal- or β-catenin signal-promoting agent is a wnt polypeptide, a dishevelled polypeptide, or a β-catenin polypeptide, or peptide mimetic thereof.

12. The method of claim 1 wherein increasing the stem cell, progenitor cell, or differentiated cell population in the vertebrate subject is a result of cell proliferation, cell homing, decreased apoptosis, self renewal, or increased cell survival.

13. A method for increasing cell or tissue regeneration in a vertebrate subject comprising,
comprising administering one or more antagonists of β-catenin-independent signaling to the vertebrate subject,
increasing in vivo a stem cell, progenitor cell, or differentiated cell population in the vertebrate subject compared to the stem cell, progenitor cell, or differentiated cell population in the vertebrate subject before treatment, to increase cell or tissue regeneration in the vertebrate subject.

14. The method of claim 13, wherein the antagonist of β-catenin-independent signaling is an antagonist of Wnt5a.

15. The method of claim 13, wherein the antagonist of β-catenin-independent signaling is an antagonist of Wnt5b.

16. The method of claim 13 wherein the antagonist of β-catenin-independent signaling increases Wnt/β-catenin signaling

17. The method of claim 13 wherein said cell or tissue regeneration occurs in bone, chondrocytes/cartilage, muscle, skeletal muscle, cardiac muscle, pancreatic cells, endothelial cells, vascular endothelial cells, adipose cells, liver, skin, connective tissue, hematopoietic stem cells, neonatal cells, umbilical cord blood cells, fetal liver cells, adult cells, bone marrow cells, peripheral blood cells, erythroid cells, granulocyte cells, macrophage cells, granulocyte-macrophage cells, B cells, T cells, multipotent mixed lineage colony types, embryonic stem cells, mesenchymal progenitor/stem cells, mesodermal progenitor/stem cells, neural progenitor/stem cells, or nerve cells.

18. The method of claim 13 wherein the vertebrate is mammalian, avian, reptilian, amphibian, osteichthyes, or chondrichthyes.

19. The method of claim 13, wherein the antagonist of β-catenin-independent signaling is a polypeptide, peptide mimetic, nucleic acid, small chemical molecule, antisense oligonucleotide, ribozyme, RNAi construct, siRNA, shRNA, or antibody.

20. The method of claim 19, wherein the antagonist of β-catenin-independent signaling is a polypeptide or peptide mimetic.

21. The method of claim 20, wherein the antagonist of β-catenin-independent signaling is a polypeptide or peptide mimetic of Wnt5a or Wnt5b.

22. The method of claim 13 wherein increasing the stem cell, progenitor cell, or differentiated cell population in the vertebrate subject is a result of cell proliferation, cell homing, decreased apoptosis, self renewal, or increased cell survival.
